(19) United States
(12) Patent Application Publication
(10) Pub. No.: US 2022/0324876 A1
(43) Pub. Date: Oct. 13, 2022

(54) 4-SUBSTITUTED PYRANO[3,4,b]PYRAZINE KAPPA AGONISTS FOR TREATING DRUG DEPENDENCY

(71) Applicant: The Rockefeller University, New York, NY (US)

(72) Inventors: Mary Jeanne KREEK, New York, NY (US); Brian REED, New York, NY (US); Eduardo BUTELMAN, New York, NY (US); Michael MILLER, New York, NY (US)

(21) Appl. No.: 17/596,123

(22) PCT Filed: Jun. 4, 2020
(86) PCT No.: PCT/US2020/036078

§ 371 Date: Dec. 3, 2021

Related U.S. Application Data
(60) Provisional application No. 62/857,921, filed on Jun. 6, 2019.

(51) Int. Cl. C07D 491/052

(52) U.S. Cl. CPC C07D 491/052

(57) ABSTRACT

1-Phenylacetyl-8-aminohexahydro-2H-pyrano[3,4-b]pyrazines of formula

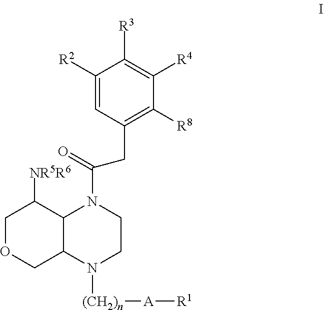

are disclosed. The compounds are kappa ligands and are useful to treat drug dependency.

### CROSS-REFERENCE TO RELATED APPLICATIONS

This application claims priority from U.S. provisional application 62/857,921, filed Jun. 6, 2019, which is incorporated herein by reference in its entirety.

### FIELD OF THE INVENTION

The invention relates to kappa opioid receptor ligands of the 1-phenylacetyl-8-aminohexahydro-2H-pyrano[3,4-b]pyrazine family that are useful to treat drug dependency.

### BACKGROUND OF THE INVENTION

One-and-a-half million current (past-month) cocaine users (12 or older) (approximately 0.6% of the U.S. population) were reported in 2014. The 2011 Drug Abuse Warning Network (DAWN) report showed that, of the nearly 1.3 million visits to emergency departments for illicit drug misuse or abuse, cocaine was involved in over 500,000 of these emergency department visits. No medication has been shown to be effective in humans for treating cocaine dependence.

The endogenous opioid system consists of the mu, delta, and kappa opioid receptors (MOP-r, DOP-r, and KOP-r, respectively), as well as the closely related non-opioid nociceptin receptor (NOP-r, also referred to as OPRL1). The endogenous opioid receptor ligands are the opioid peptides, collectively, which all share the common N-terminal amino acid sequence motif, Tyr-Gly-Gly-Phe-Met/Leu. Three separate genes encode for the opioid peptide precursors, proenkephalin, prodynorphin, and proopiomelanocortin, which are processed to the enkephalins, the dynorphins, and beta-endorphin, respectively (note, proopiomelanocortin also encodes for other peptides with non-opioidergic function). Nociceptin (also referred to as orphanin FQ) is closely related to the opioid peptides, with especially high homology to dynorphin A (1-17), but with a modified N-terminus which distinguishes its activity from the opioid peptides.

Exposure to cocaine, which inhibits the biogenic amine neurotransmitter transporters, acutely causes increased extracellular dopamine, serotonin, and norepinephrine, and also results in changes in components of the endogenous opioid system. Acutely, cocaine results in increased gene expression of dynorphin in the dorsal and ventral striatum, in animal models. Chronic cocaine exposure also results in changes in mu and kappa opioid receptor binding. Similar alterations have been detected in human postmortem brain following cocaine abuse or dependence. Kappa opioid receptor/dynorphin dysfunction has been observed following experimental stress in animals, with accompanying depressant-like behavioral effects. Additionally, PET imaging has shown brain KOP-r populations to be altered in people exhibiting symptoms of trauma, including anhedonia or dysphoria and anxiety.

Full kappa agonists have the ability to block the rewarding effects of cocaine, but by themselves they have been shown to be aversive [see Zhang et al. Psychopharmacology 179(3): 551-558 (2005)]. Similarly, dysphoria and psychotomimetic/hallucinogenic effects result from KOP-r agonist administration in humans [see Pfeiffer et al. Science 233(4765): 774-776 (1986)]. Kappa antagonists have been shown to block stress induced reinstatement to cocaine seeking in animal self-administration models, but with no effect on drug-induced reinstatement.

Although multiple selective KOP-r antagonists (with no agonistic efficacy, and full blockade) have been identified to date, the only selective partial or differentially efficacious G-protein/beta-arrestin signaling biased KOP-r agonists that have been tested in animal models of drug addiction are found in PCT/US2018/064422, which has overlapping inventorship with the instant application.

### SUMMARY OF THE INVENTION

It has now been found that 1-phenylacetyl-8-aminohexahydro-2H-pyrano[3,4-b]pyrazine derivatives are KOP-r ligands with differential agonistic activity, making them useful to treat dependence on cocaine as well as other psychostimulants and alcohol, as well as mood disorders.

In one aspect, the invention relates to compounds of Formula I

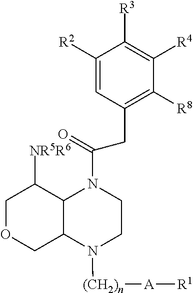

wherein
A is chosen from —(C═))—, —CH₂—, —CH(OH)—, —(C═O)NH—, —SO₂—, and a direct bond
n is 0, 1, or 2;
R¹ is chosen from cyano, hydroxy(C₁-C₆)hydrocarbyl, (C₁-C₆)oxaalkyl, fluoro(C₁-C₆)alkyl, cyano, —COOH, —SO₂NH(C₁-C₆)hydrocarbyl, —SO₂N[(C₁-C₆)hydrocarbyl]₂, and optionally-substituted heterocyclyl, wherein substituents on said heterocycle are chosen from (C₁-C₇)hydrocarbyl, (C₁-C₃)alkoxy, fluoro(C₁-C₃)alkyl, hydroxy, and oxo;
or, when A is —(C═O)NH—, R¹ may additionally be hydrogen or (C₁-C₆)alkyl;
or, when n is other than zero, R¹ may additionally be —SO₂(C₁-C₆)hydrocarbyl
R², R³, R⁴, and R⁸ are chosen independently from hydrogen, halogen, (C₁-C₄)alkyl, fluoro(C₁-C₄)alkyl, cyano, nitro, —SO₃H and —N⁺HR⁵R⁶; and
R⁵ and R⁶ are chosen from (C₁-C₁₀)hydrocarbyl, optionally substituted with fluoro, or, taken together with the nitrogen to which they are attached, R⁵ and R⁶ form a five-, six- or seven-membered non-aromatic heterocycle, which may be optionally substituted with fluoro or (C₁-C₄)alkyl.

In another aspect, the invention relates to a method for activating a kappa opioid receptor. The method comprises contacting a kappa opioid receptor with a compound as described herein.

In another aspect, the invention relates to a method for treating addictive diseases, i.e. addiction and substance abuse disorders. The method comprises administering to a patient a compound as described herein.

In another aspect, the invention relates to a method for treating mood disorders. The method comprises administering to a patient a compound as described herein.

In another aspect, the invention relates to pharmaceutical compositions comprising a pharmaceutically acceptable carrier and a compound as described herein.

### DETAILED DESCRIPTION OF THE INVENTION

In one aspect, the invention relates to compounds of formula I:

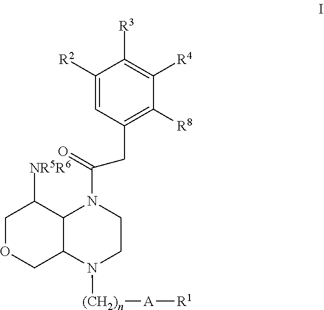

In these compounds, A is chosen from —(C═O)—, —CH₂—, —CH(OH)—, —(C═O)NH—, —SO₂—, and a direct bond. In some embodiments, n is zero and A is —CH₂— or —C(═O)—. In other embodiments, n is one and A is —CH₂— or —CH(OH)—.

R¹ may be cyano, hydroxy(C₁-C₆)hydrocarbyl, (C₁-C₆)oxaalkyl, fluoro(C₁-C₆)alkyl, cyano, —COOH, —SO₂NH(C₁-C₆)hydrocarbyl, —SO₂N[(C₁-C₆)hydrocarbyl]₂, and optionally-substituted heterocyclyl. Additionally, when A is —(C═O)NH—, R¹ may be hydrogen or (C₁-C₆)alkyl; and when n is other than zero, R¹ may additionally be —SO₂(C₁-C₆)hydrocarbyl.

In some embodiments, R¹ is optionally-substituted heterocyclyl. In some embodiments, R¹ is optionally-substituted heterocyclyl and (1) n is zero and A is —CH₂— or —C(═O)—; or (2) n is one and A is —CH₂— or —CH(OH)—. In these compounds, the optionally-substituted heterocyclyl may be chosen from tetrahydrofuran, isoxazole, oxazole, oxetane, pyrazole, pyridine, oxadiazole, pyrimidine, pyrrolidine, tetrahydropyran, and tetrahydrothiopyran 1,1-dioxide. The heterocycle may be unsubstituted or, in some embodiments, substituted with methyl and/or hydroxy. Particularly when the heterocycle is chosen from tetrahydrofuran, oxetane, and tetrahydropyran, it may be substituted with hydroxy at the position of attachment of the heterocycle to A, for example as in rac-5A139:

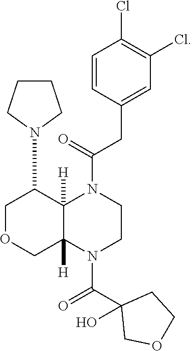 [SMILES: O=C(Cc1ccc(Cl)c(Cl)c1)N1CCN(C(=O)C2(O)CCOC2)[C@@H]2COC[C@H](N3CCCC3)[C@H]21]

In some embodiments in which R¹ is optionally-substituted heterocyclyl, the heterocycle may be chosen from isoxazole, oxazole, pyrazole, pyridine, oxadiazole, pyrimidine, and pyrrolidine, which may be unsubstituted or, in some embodiments, substituted with methyl.

In other embodiments, R¹ is fluoro(C₃-C₁₀)hydrocarbyl, and in one subset of these, n is one, A is a direct bond and R¹ is chosen from mono-, di-, or trifluoro(C₂-C₆)alkyl and fluorophenyl.

In other embodiments, R¹ is one of the following: (a) —SO₂N[(C₁-C₆)alkyl]₂; (b) hydroxy(C₁-C₆)alkyl or hydroxy(C₃-C₆)cycloalkyl, particularly hydroxymethyl, hydroxyethyl, hydroxypropyl, hydroxycyclopropyl, hydroxycyclobutyl, or hydroxycyclopentyl; (c) methoxy(C₁-C₆)alkyl; (d) cyano; (e) —C(═O)NH₂; —COOH; (g) —SO₂CH₃; (h) —SO₂(CH₂)ₘOH, or —SO₂(CH₂)mOCH₃, wherein m is two or three.

In some embodiments, two of R², R³, R⁴, and R⁸ are hydrogen and the remaining two are chosen from halogen, (C₁-C₄)alkyl, fluoro(C₁-C₄)alkyl, and cyano. In particular embodiments, R² and R⁸ are hydrogen, and R³ and R⁴ are halogen or trifluoromethyl. In other embodiments, three of R², R³, and R⁴ are hydrogen and the remaining one is chosen from chloro, fluoro, trifluoromethyl, and cyano.

In some embodiments, R⁵ and R⁶ form a five-, six- or seven-membered non-aromatic heterocycle, which may be optionally substituted with fluoro or (C₁-C₄)alkyl. In particular embodiments, —NR⁵R⁶ is

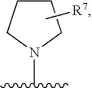

wherein R⁷ is chosen from hydrogen, fluoro and (C₁-C₃)alkyl.

In some embodiments, the ring junction of the octahydro-1H-pyrano[3,4-b]pyrazine is trans and —NR⁵R⁶ is cis to its adjacent hydrogen at the ring junction:

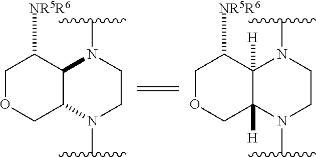

Throughout this specification the terms and substituents retain their definitions.

C₁ to C₁₀ hydrocarbon includes alkyl, cycloalkyl, polycycloalkyl, alkenyl, alkynyl, aryl and combinations thereof. Examples include benzyl, phenethyl, cyclohexylmethyl, adamantyl, camphoryl and naphthylethyl. Hydrocarbyl refers to any substituent comprised of hydrogen and carbon as the only elemental constituents. Aliphatic hydrocarbons are hydrocarbons that are not aromatic; they may be saturated or unsaturated, cyclic, linear or branched. Examples of aliphatic hydrocarbons include isopropyl, 2-butenyl, 2-butynyl, cyclopentyl, norbornyl, etc. Aromatic hydrocarbons include benzene (phenyl), naphthalene (naphthyl), anthracene, etc.

Unless otherwise specified, alkyl (or alkylene) is intended to include linear or branched saturated hydrocarbon structures and combinations thereof. Alkyl refers to alkyl groups from 1 to 20 carbon atoms, preferably 1 to 10 carbon atoms, more preferably 1 to 6 carbon atoms. Examples of alkyl groups include methyl, ethyl, propyl, isopropyl, n-butyl, s-butyl, t-butyl and the like.

Cycloalkyl is a subset of hydrocarbon and includes cyclic hydrocarbon groups of from 3 to 8 carbon atoms. Examples of cycloalkyl groups include cyclopropyl, cyclobutyl, cyclopentyl, norbornyl and the like.

Unless otherwise specified, the term “carbocycle” is intended to include ring systems in which the ring atoms are all carbon but of any oxidation state. Thus (C₃-C₁₀) carbocycle refers to both non-aromatic and aromatic systems, including such systems as cyclopropane, benzene and cyclohexene; (C₈-C₁₂) carbopolycycle refers to such systems as norbornane, decalin, indane and naphthalene. Carbocycle, if not otherwise limited, refers to monocycles, bicycles and polycycles.

Heterocycle means an aliphatic or aromatic carbocycle residue in which from one to four carbons is replaced by a heteroatom selected from the group consisting of N, O, and S. The nitrogen and sulfur heteroatoms may optionally be oxidized, and the nitrogen heteroatom may optionally be quaternized. Unless otherwise specified, a heterocycle may be non-aromatic (heteroaliphatic) or aromatic (heteroaryl). Examples of heterocycles include pyrrolidine, pyrazole, pyrrole, indole, quinoline, isoquinoline, tetrahydroisoquinoline, benzofuran, benzodioxan, benzodioxole (commonly referred to as methylenedioxyphenyl, when occurring as a substituent), tetrazole, morpholine, thiazole, pyridine, pyridazine, pyrimidine, thiophene, furan, oxazole, oxazoline, isoxazole, dioxane, tetrahydrofuran and the like. Examples of heterocyclyl residues include piperazinyl, piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, benzoxazolyl, tetrahydrofuryl, tetrahydropyranyl, thienyl (also historically called thiophenyl), benzothienyl, thiamorpholinyl, oxadiazolyl, triazolyl and tetrahydroquinolinyl.

Hydrocarbyloxy refers to groups of from 1 to 20 carbon atoms, preferably 1 to 10 carbon atoms, more preferably 1 to 6 carbon atoms attached to the parent structure through an oxygen. Alkoxy is a subset of hydrocarbyloxy and includes groups of a straight or branched configuration. Examples include methoxy, ethoxy, propoxy, isopropoxy and the like. Lower-alkoxy refers to groups containing one to four carbons. The term “halogen” means fluorine, chlorine, bromine or iodine atoms.

Unless otherwise specified, acyl refers to formyl and to groups of 1, 2, 3, 4, 5, 6, 7 and 8 carbon atoms of a straight, branched, cyclic configuration, saturated, unsaturated and aromatic and combinations thereof, attached to the parent structure through a carbonyl functionality. Examples include acetyl, benzoyl, propionyl, isobutyryl and the like. Lower-acyl refers to groups containing one to four carbons.

Oxaalkyl refers to alkyl residues in which one or more carbons (and their associated hydrogens)—other than the carbon at the point of attachment of the residue—have been replaced by oxygen. In other words, it refers to carbon-attached oxaalkyl. Examples include methoxyethyl, ethoxyethyl, methoxypropyl and the like. As used herein it is not intended to encompass alkoxy residues, wherein the oxygen is the point of attachment. Oxaalkyl refers to compounds in which the oxygen is bonded via a single bond to its adjacent carbon atoms (forming ether bonds); it does not refer to doubly bonded oxygen, as would be found in carbonyl groups.

As used herein, the term “optionally substituted” may be used interchangeably with “unsubstituted or substituted”. The term “substituted” refers to the replacement of one or more hydrogen atoms in a specified group with a specified radical. For example, substituted alkyl, aryl, cycloalkyl, heterocyclyl etc. refer to alkyl, aryl, cycloalkyl, or heterocyclyl wherein one or more H atoms in each residue are replaced with halogen, haloalkyl, alkyl, acyl, alkoxyalkyl, hydroxy lower alkyl, carbonyl, phenyl, heteroaryl, benzenesulfonyl, hydroxy, lower alkoxy, haloalkoxy, oxaalkyl, carboxy, alkoxycarbonyl [—C(═O)O-alkyl], alkoxycarbonylamino [HNC(═O)O-alkyl], aminocarbonyl (also known as carboxamido) [—C(═O)NH_{2],} alkylaminocarbonyl [—C(═O)NH-alkyl], cyano, acetoxy, nitro, amino, alkylamino, dialkylamino, (alkyl)(aryl)aminoalkyl, alkylaminoalkyl (including cycloalkylaminoalkyl), dialkylaminoalkyl, dialkylaminoalkoxy, heterocyclylalkoxy, mercapto, alkylthio, sulfoxide, sulfone, sulfonylamino, alkylsulfinyl, alkyl sulfonyl, acylaminoalkyl, acylaminoalkoxy, acylamino, amidino, aryl, benzyl, heterocyclyl, heterocyclylalkyl, phenoxy, benzyloxy, heteroaryloxy, hydroxyimino, alkoxyimino, oxaalkyl, aminosulfonyl, trityl, amidino, guanidino, ureido, benzyloxyphenyl, and benzyloxy. In one embodiment, 1, 2, or 3 hydrogen atoms are replaced with a specified radical. In the case of alkyl and cycloalkyl, more than three hydrogen atoms can be replaced by fluorine; indeed, all available hydrogen atoms could be replaced by fluorine.

Substituents Rⁿ are generally defined when introduced and retain that definition throughout the specification and in all independent claims.

Preparation of compounds can involve the protection and deprotection of various chemical groups. The need for protection and deprotection, and the selection of appropriate protecting groups, can be readily determined by one skilled in the art. Suitable groups for that purpose are discussed in standard textbooks in the field of chemistry, such as Protective Groups in Organic Synthesis by T. W.Greene and P. G. M. Wuts [John Wiley & Sons, New York, 1999], in Protecting Group Chemistry, 1^{st} Ed., Oxford University Press, 2000; and in March's Advanced Organic chemistry: Reactions, Mechanisms, and Structure, 5^{th} Ed., Wiley-Interscience Publication, 2001.

The compounds described herein contain three asymmetric centers and may thus give rise to enantiomers, diastereomers, and other stereoisomeric forms which may be defined in terms of absolute stereochemistry as (R)- or (S)-. The present invention is meant to include all such possible isomers as racemates, optically pure forms and intermediate mixtures. Optically active isomers may be prepared using homo-chiral synthons or homo-chiral reagents, or optically resolved using conventional techniques. All tautomeric forms are intended to be included. The graphic representations of racemic, ambiscalemic and scalemic or enantiomerically pure compounds used herein are taken from Maehr J. Chem. Ed. 62, 114-120 (1985): simple, single bond lines convey connectivity only and no stereochemical implication; solid and broken wedges are used to denote the absolute configuration of a chiral element; wavy lines indicate explicit disavowal of any stereochemical implication which the bond it represents could generate; solid and broken bold lines are geometric descriptors indicating the relative configuration shown but do not denote absolute configurations; and wedge outlines and dotted or broken lines denote enantiomerically pure compounds of indeterminate absolute configuration. Enantiomerically pure means greater than 80 ee, and preferably greater than 90 ee.

For example, the generic structure depicting the compounds of the invention:

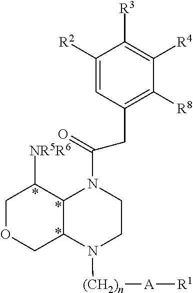

contains three asymmetric centers (labeled with asterisks). In one embodiment, the relative stereochemistry of the diastereomer can be represented as:

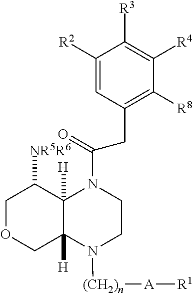

This representation implies that the material is a mixture of isomers [(4aS,8R,8aR)-8-cyclopentyl-octahydro-1H-pyrano[3,4-b]pyrazine and (4aR,8S,8aS)-8-cyclopentyl-octahydro-1H-pyrano[3,4-b]pyrazine] in which the ring junction of the octahydro-1H-pyrano[3,4-b]pyrazine is trans and —NR⁵R⁶ is cis to its adjacent hydrogen at the ring junction.

As used herein, the terms “treatment” or “treating,” or “palliating” or “ameliorating” refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological systems associated with the underlying disorder such that an improvement is observed in the patient, notwithstanding that the patient may still be afflicted with the underlying disorder.

As used herein, and as would be understood by the person of skill in the art, the recitation of “a compound”—unless expressly further limited—is intended to include salts of that compound. In a particular embodiment, the term “compound of formula” refers to the compound or a pharmaceutically acceptable salt thereof.

The term “pharmaceutically acceptable salt” refers to salts prepared from pharmaceutically acceptable non-toxic acids or bases including inorganic acids and bases and organic acids and bases. When the compounds of the present invention are basic—as they are in most cases—salts may be prepared from pharmaceutically acceptable non-toxic acids including inorganic and organic acids. Suitable pharmaceutically acceptable acid addition salts for the compounds of the present invention include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When the compounds contain an acidic functionality (e.g. —SO₃H), suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms.

Also provided herein is a pharmaceutical composition comprising a compound disclosed above, or a pharmaceutically acceptable salt thereof, together with one or more pharmaceutically carriers thereof and optionally one or more other therapeutic ingredients. The carrier(s) must be “acceptable” in the sense of being compatible with the other ingredients of the formulation and not deleterious to the recipient thereof.

The formulations include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association a compound of formula I or a pharmaceutically acceptable salt thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.\

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

In general, compounds of formula I can be prepared as described in PCT/US2018/064422 and depicted in the general scheme below.

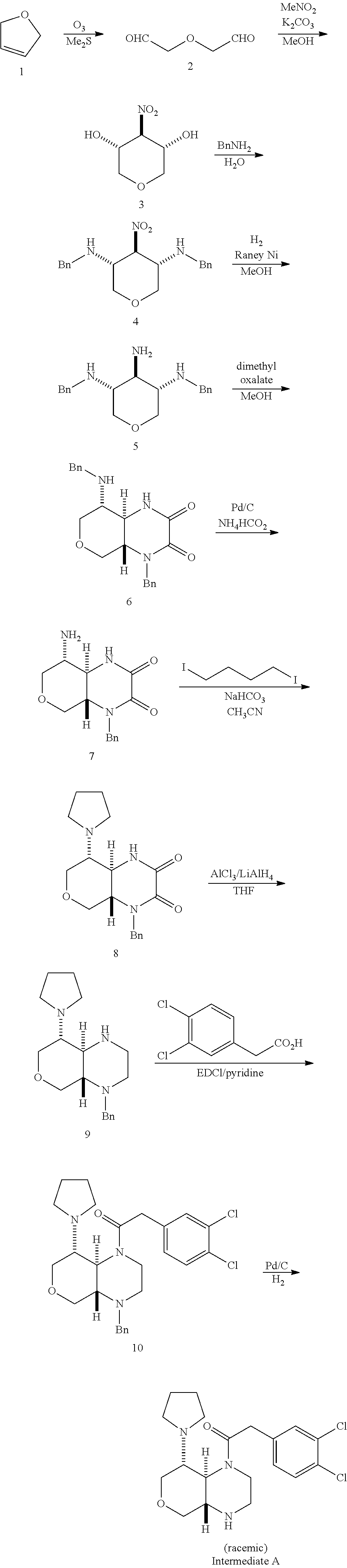

When it is desired that R², R³, R⁴, and R⁸ be other than as shown, the appropriate phenylacetic acid may be substituted for 3,4-dichlorophenyl acetic acid in steps 9-10 above. Similarly, when it is desired that R⁵ and R⁶ be other than as shown, the appropriate alkylating agent(s) may be used in place of diiodobutane. The intermediate A may then be alkylated, acylated, etc. with the appropriate moiety to attach —(CH₂)ₙ-A-R¹ by methods well-known in the art.

### Preparation of Intermediate 2

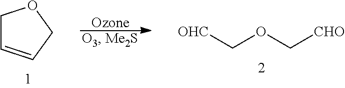

A solution of 2,5-dihydrofuran (50.00 g, 713 mmol, 53.8 mL, 1.00 eq) in MeOH (20 mL) and dichloromethane (DCM) (200 mL) was subjected to ozone (34.2 g, 713 mmol, 1 eq) (15 Psi) at −78° C. for 2 h. Dimethylsulfide (133 g, 2.14 mol, 156 mL, 3 eq) was added, and the resulting solution was stirred at 25° C. for 14 hr. The reaction mixture was filtered and concentrated under reduced pressure. The residue consisted of the crude product, 2-(2-oxoethoxy)acetaldehyde (70 g). The material was used without further purification.

### Preparation of Intermediate 3

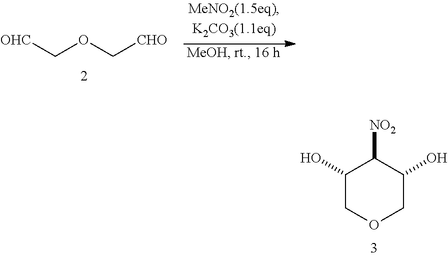 [SMILES: O=CCOCC=O ; "MeOH, rt., 16 h" ; O=[N+]([O-])[C@H]1[C@H](O)COC[C@@H]1O]

To a solution of 2-(2-oxoethoxy)acetaldehyde (70 g, 686 mmol, 1 eq) in MeOH (500 mL) was added nitromethane (65.7 g, 1.08 mol, 58.2 mL, 1.57 eq), K₂CO₃ (104 g, 754 mmol, 1.1 eq). The resulting mixture was stirred for 3 hr at 0° C. followed by 13 hrs at 25° C. The reaction mixture was filtered and concentrated under reduced pressure to furnish 4-nitrotetrahydropyran-3,5-diol (72 g). The material was used directly in the next step.

### Preparation of Intermediate 4

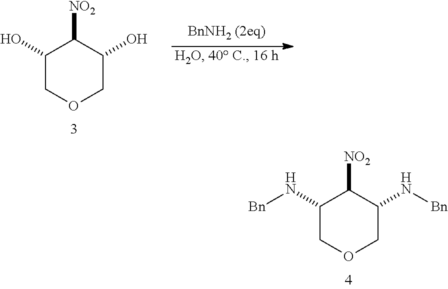 [SMILES: O=[N+]([O-])[C@H]1[C@H](O)COC[C@@H]1O ; "H2O, 40° C., 16 h" ; O=[N+]([O-])[C@@H]1[C@@H](NCc2ccccc2)COC[C@H]1NCc1ccccc1]

To a solution of 4-nitrotetrahydropyran-3,5-diol (15.0 g, 92.0 mmol, 1 eq) in H₂O (110 mL) was added benzylamine (19.7 g, 184 mmol, 20.10 mL, 2 eq). The solution was stirred at 40° C. for 16 hr. The reaction mixture was filtered and concentrated under reduced pressure. The residue was purified by preparative HPLC to furnish N3,N5-dibenzyl-4-nitro-tetrahydropyran-3,5-diamine (20.00 g, crude) LC/MS m/z 342.2 (M+H).

### Preparation of Intermediate 5

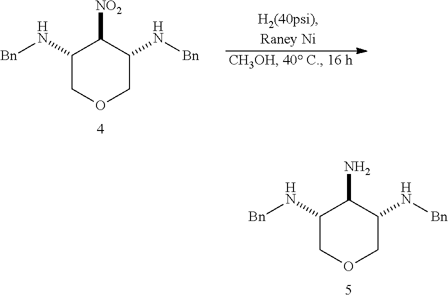 [SMILES: O=[N+]([O-])[C@@H]1[C@@H](NCc2ccccc2)COC[C@H]1NCc1ccccc1 ; "CH3OH, 40° C., 16 h" ; "H2(40psi),Raney Ni" ; N[C@@H]1[C@@H](NCc2ccccc2)COC[C@H]1NCc1ccccc1]

To a solution of N3,N5-dibenzyl-4-nitro-tetrahydropyran-3,5-diamine (5.00 g, 14.7 mmol, 1 eq) in MeOH (10 mL) was added Raney-Ni (1.25 g, 14.7 mmol, 1 eq). The mixture was stirred under H₂ (45 Psi) at 25° C. for 16 hrs. The reaction mixture was filtered and concentrated under reduced pressure to furnish N3,N5-dibenzyltetrahydropyran-3,4,5-triamine (5.30 g, crude). ¹H-NMR (400 MHz, DMSO-d₆): δ 7.36-7.19 (10H, m), 3.91-3.78 (2H, m), 3.78-3.70 (2H, m), 3.65 (2H, d, J=13.7 Hz), 2.88 (2H, t, J=10.5 Hz), 2.29-1.90 (6H, m). LC/MS m/z 312.3 (M+H). The material was used directly in the next step without further purification.

### Preparation of Intermediate 6

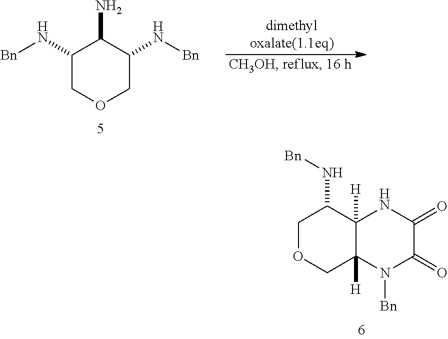 [SMILES: N[C@@H]1[C@@H](NCc2ccccc2)COC[C@H]1NCc1ccccc1 ; "CH3OH, reflux, 16 h" ; "dimethyloxalate(1.1eq)" ; O=C1N[C@@H]2[C@@H](NCc3ccccc3)COC[C@H]2N(Cc2ccccc2)C1=O]

To a solution of N3,N5-dibenzyltetrahydropyran-3,4,5-triamine (3.10 g, 9.95 mmol, 1 eq) in MeOH (30 mL) was added dimethyl oxalate (1.17 g, 9.95 mmol, 1 eq). The solution was stirred at 66° C. for 16 hr. The reaction mixture was filtered and dried in vacuo to furnish 4-benzyl-8-(benzylamino)-1,4a,5,7,8,8a-hexahydropyrano[3,4-b]pyrazine-2,3-dione (2.70 g, 6.80 mmol, 68%) LC/MS m/z=366.3 (M+H).

### Preparation of Intermediate 7

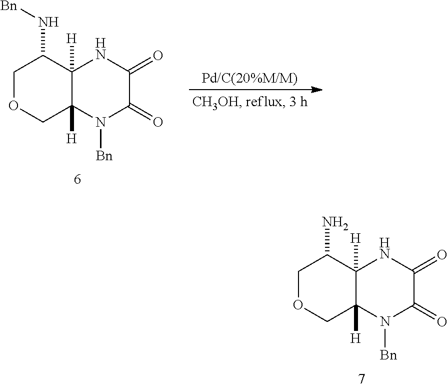 [SMILES: O=C1N[C@@H]2[C@@H](NCc3ccccc3)COC[C@H]2N(Cc2ccccc2)C1=O ; "Pd/C(20%M/M)" ; N[C@H]1COC[C@@H]2[C@@H]1NC(=O)C(=O)N2Cc1ccccc1]

To a solution of 4-benzyl-8-(benzylamino)-1,4a,5,7,8,8a-hexahydropyrano[3,4-b]pyrazine-2,3-dione (2.70 g, 7.39 mmol, 1 eq) in MeOH (50 mL) was added Pd/C (1.35 g, 10 wt % Pd) and ammonium formate (4.66 g, 73.9 mmol, 10 eq). The mixture was stirred at 65° C. for 3 hr. The reaction mixture was filtered and concentrated under reduced pressure to furnish 8-amino-4-benzyl-1,4a,5,7,8,8a-hexahydropyrano [3,4-b]pyrazine-2,3-dione (1.60 g, 5.81 mmol, 78% yield). ¹H-NMR (400 MHz, methanol-d₄): δ 7.39-7.33 (2H, m), 7.32-7.24 (3H, m), 4.95-4.89 (2H, m), 4.55 (1H, d, J=15.9 Hz), 4.13 (1H, dd, J=4.3, 10.9 Hz), 3.87 (1H, dd, J=5.0, 11.2 Hz), 3.69 (1H, dt, J=4.3, 10.7 Hz), 3.43 (1H, t, J=10.4 Hz), 3.29-3.21 (1H, m), 3.05 (1H, t, J=11.0 Hz), 2.84-2.75 (1H, m). LC/MS m/z 276.3 (M+H).

### Preparation of Intermediate 8

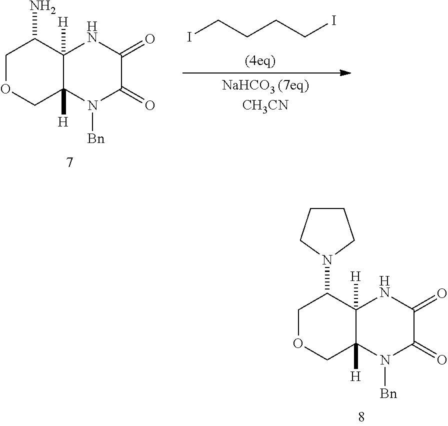

To a solution of 8-amino-4-benzyl-1,4a,5,7,8,8a-hexahydropyrano[3,4-b]pyrazine-2,3-dione (2.00 g, 7.26 mmol, 1 eq) in CH₃CN (50 mL) was added NaHCO₃ (4.15 g, 49.4 mmol) and 1,4-diiodobutane (9.00 g, 29.0 mmol, 3.81 mL, 4 eq). The mixture was stirred at 82° C. for 18 hrs. The reaction mixture was filtered and concentrated under reduced pressure to furnish 4-benzyl-8-pyrrolidin-1-yl-1,4a,5,7,8,8a-hexahydropyrano [3,4-b]pyrazine-2,3-dione (3.36 g, crude). ¹H-NMR (400 MHz, methanol-d₄): δ 7.39-7.33 (2H, m), 7.32-7.24 (3H, m), 4.93 (1H, d, J=15.8 Hz), 4.51 (1H, d, J=15.8 Hz), 4.14-4.00 (2H, m), 3.79-3.67 (2H, m), 3.45 (1H, t, J=11.0 Hz), 3.28-3.23 (1H, m), 2.97 (1H, dt, 10.5 Hz), 2.84-2.76 (2H, m), 2.75-2.67 (2H, m), 1.83-1.71 (4H, m). LC/MS m/z 330.0 (M+H).

### Preparation of Intermediate 9

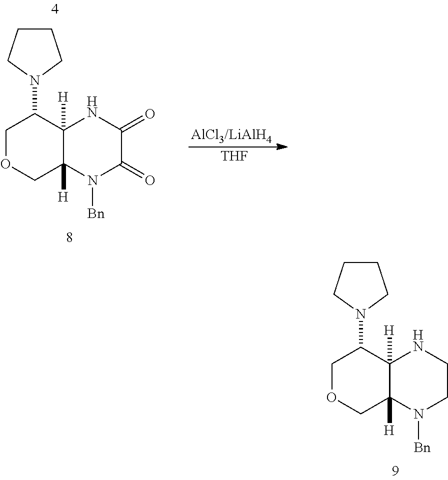 [SMILES: "THF" ; O=C1N[C@H]2[C@@H](COC[C@@H]2N2CCCC2)N(Cc2ccccc2)C1=O ; "AlCl3/LiAlH4" ; c1ccc(CN2CCN[C@H]3[C@H]2COC[C@@H]3N2CCCC2)cc1]

To a solution of AlCl₃ (1.19 g, 8.89 mmol, 1.83 eq) in THF (30 mL) was added LiAlH₄ (1.03 g, 27.0 mmol, 5.56 eq) at 0° C. The mixture was stirred at 25° C. for 30 min. To the solution was added 4-benzyl-8-pyrrolidin-1-yl-1,4a,5,7,8,8a-hexahydropyrano[3,4-b]pyrazine-2,3-dione (1.60 g, 4.86 mmol, 1 eq) at 0° C. The mixture was stirred at 0° C. for 1 hr and then warmed to 25° C. for 30 min. The soluton was adjusted to pH=8 by addition of NaOH_{(aq.)} (2 M). The mixture was extracted with ethyl acetate (3×30 mL).The organic layer was dried (Na₂SO₄), filtered, and concentrated in vacuo to furnish 4-Benzyl -8-pyrrolidin-1-yl-1,2,3,4a,5,7,8,8a-octahydropyrano[3,4-b]pyrazine (850 mg, 2.82 mmol, 58% yield). LC/MS m/z 302.3 (M+H).

### Preparation of Intermediate 10

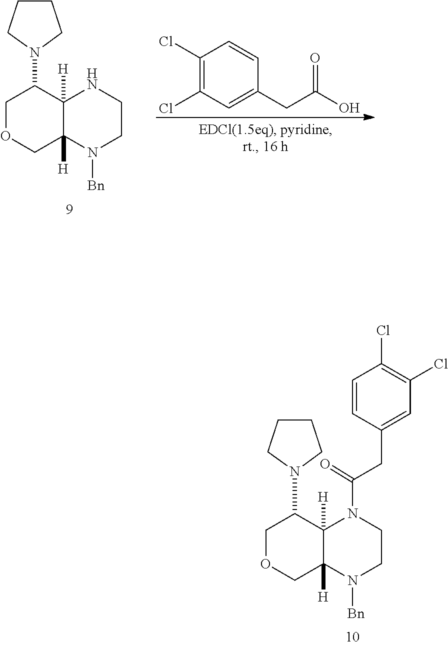 [SMILES: c1ccc(CN2CCN[C@H]3[C@H]2COC[C@@H]3N2CCCC2)cc1 ; O=C(O)Cc1ccc(Cl)c(Cl)c1 ; O=C(Cc1ccc(Cl)c(Cl)c1)N1CCN(Cc2ccccc2)[C@@H]2COC[C@H](N3CCCC3)[C@H]21]

To a solution of 2-(3,4-dichlorophenyl)acetic acid (591 mg, 2.88 mmol, 1.1 eq) in pyidine (20 mL) was added 1-ethyl-3-(3-dimethylaminopropyl)carbodiimide (754 mg, 3.93 mmol, 1.5 eq). The solution was stirred at 25° C. for 30 min. 4-Benzyl-8-pyrrolidin-1-yl-1,2,3,4a,5,7,8,8a-octahydropyrano[3,4-b]pyrazine (790.00 mg, 2.62 mmol, 1.00 eq) was added to the solution. The solution was stirred at 25° C. for 15.5 hrs. The reaction mixture was diluted with 40 mL H₂O, and the resulting mixture was extracted with ethyl acetate (20 mL×3). The combined organic layers were dried over Na₂SO₄, filtered, and concentrated under reduced pressure to furnish 1-[4-benzyl-8-pyrrolidin-1-yl-3,4a,5,7,8,8a-hexahydro-2H-pyrano(3,4-b)pyrazin-1-yl]-2-(3,4- dichlorophenyl)ethanone (730 mg, 1.49 mmol, 57% yield). LC/MS m/z 488.0 (M+H).

### Preparation of Intermediate A

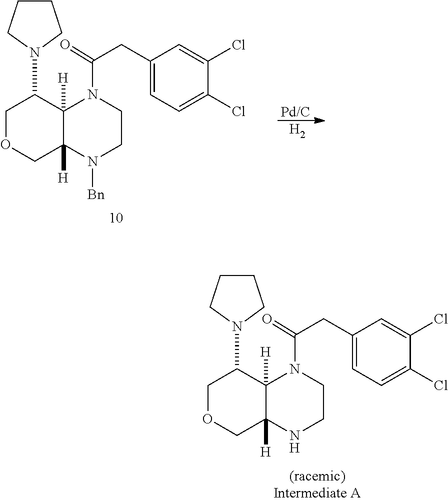 [SMILES: O=C(Cc1ccc(Cl)c(Cl)c1)N1CCN(Cc2ccccc2)[C@@H]2COC[C@H](N3CCCC3)[C@H]21 ; "H2" ; "Pd/C" ; O=C(Cc1ccc(Cl)c(Cl)c1)N1CCN[C@@H]2COC[C@H](N3CCCC3)[C@H]21]

To a solution of palladium on carbon (1.42 g, 10% wt % Pd) in THF (20 mL) and H₂O (20 mL) was added 1-[4-benzyl-8-pyrrolidin-1-yl-3,4a,5,7,8,8a-hexahydro-2H-pyrano(3,4-b)pyrazin-1-yl]-2-(3,4- dichlorophenyl)ethanone (710 mg, 1.45 mmol, 1 eq) and HC₁ (14.00 mL, 1.0 M in water). The mixture was stirred at 25° C. for 40 min under H₂ (1 bar). The reaction mixture was quenched by addition of 40 mL saturated, aqueous NaHCO₃ at 25° C. The mixture was filtered. The filtrate was extracted with DCM (50 mL×3). The combined organic layers were dried over Na₂SO₄, filtered, and concentrated under reduced pressure to furnish 1-[8-pyrrolidin-1-yl-2,3,4,4a,5,7,8,8a-octahydropyrano(3,4-b) pyrazin-1-yl]-2-(3,4-dichlorophenyl)ethanone (380 mg, 927 μmol, 64% yield). ¹H-NMR (400 MHz, methanol-d₄): δ 7.48-7.44 (2H, m), 7.23 (1H, dd, J=2.1, 8.2 Hz), 4.07 (1H, dd, J=4.5, 11.2 Hz), 3.97(1H, m, J=13.6 Hz), 3.91-3.80 (2H, m), 3.69 (1H, m, J=15.6 Hz), 3.35 (1H, s), 3.28-3.18 (2H, m), 3.18-3.09 (1H, m),3.08-2.90 (4H, m), 2.70 (5H, m), 2.16 (1H, s), 1.72-1.67 (4H, m). LC/MS m/z 398.2 (M+H).

As shown in General Scheme A, analogs can be prepared according to known methods to those versed in the art using rac-Intermediate A. For example, amide analogs, such as rac-GA, can be prepared using standard amide bond forming reactions, e.g. acid chlorides and/or carboxylic acid/amide coupling reagent such as HATU or EDCI. Alkylated analogs, such as rac-GB, can be prepared using the appropriate alkylating agent, for example (X—CH₂—(CH₂)ₙR¹ and base (TEA) or reductive amination with an aldehyde (R¹A(CH₂)ₙCHO) and reducing reagent (Na(AcO)₃BH or NaBH₃CN).

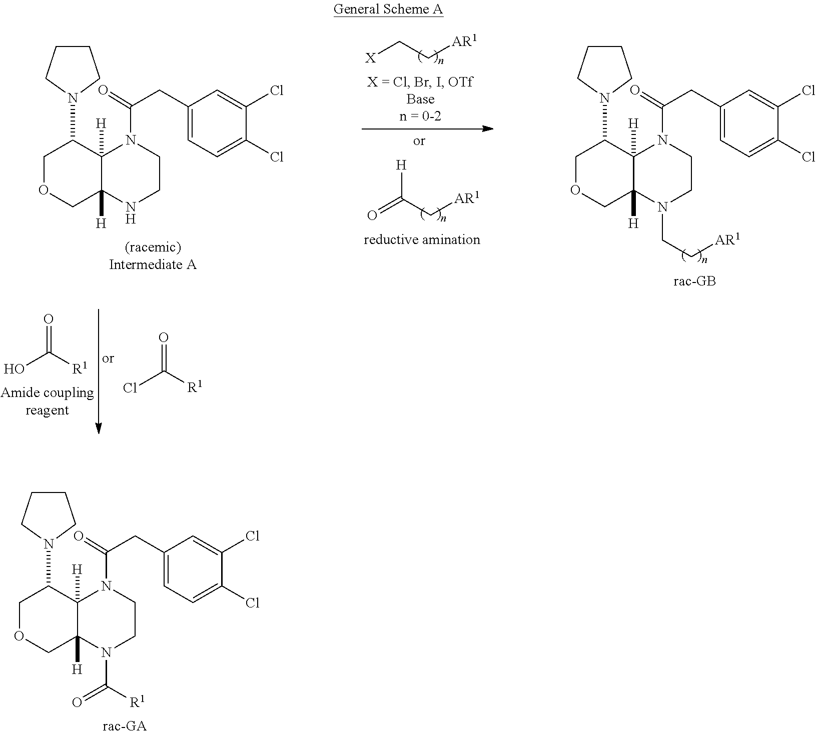

Specific examples of the invention can be prepared by the procedures shown below. The following abbreviations are used in the synthetic preparations and schemes: THF (tetrahydrofuran), EDCI (3-(Ethyliminomethyleneamino)-N,N-dimethylpropan-1-amine), HATU (1-[Bis(dimethylamino)methylene]-1H-1,2,3-triazolo[4,5-b]pyridinium 3-oxide hexafluorophosphate), TEA (triethylamine), ACN (acetonitrile), DCE (1,2-dichloroethane), DMF (N,N-dimethylformamide), DAST (diethylaminosulfur trifluoride), DCM (dichloromethane), NMM (N-methylmorpholine), DIPEA (N,N-diisopropylethylamine), DMAP (4-dimethylaminopyridine), IPA (isopropyl alcohol).

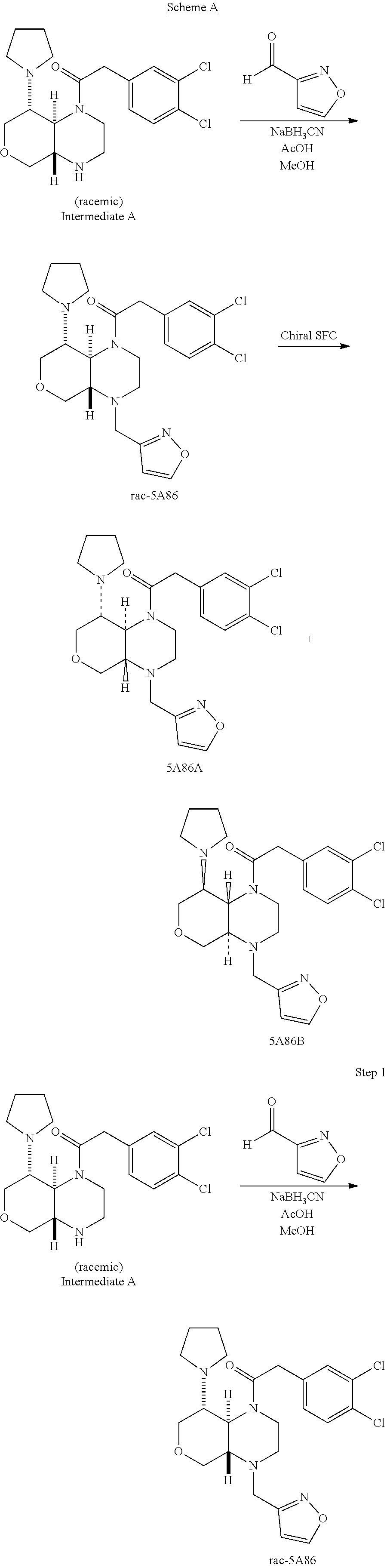

To a solution of 2-(3,4-dichlorophenyl)-1-(8-pyrrolidin-1-yl-2,3,4,4a,5,7,8,8a-octahydropyrano[3,4-b]pyrazin-1-yl)ethanone (300 mg, 753 μmol, 1 eq) in MeOH (30 mL) was added isoxazole-3-carbaldehyde (731 mg, 7.53 mmol, 10 eq) and HOAc (4.5 mL). The mixture was stirred at 20° C. for 0.5 hr. Sodium cyanoborohydride (473 mg, 7.53 mmol, 10 eq) was added to reaction. The mixture was stirred at 50° C. for 11.5 hr. The reaction mixture was concentrated under reduced pressure. The residue was diluted with H₂O (20 mL) and extracted with ethyl acetate (20 mL×3). The combined organic layers were dried over Na₂SO₄, filtered, and concentrated. The residue was purified by preparative-HPLC (column: Phenomenex Luna C₁₈ 200 mm×40 mm×10 μm; mobile phase: [water(0.05% HCl)-ACN]; B%: 20%-50%, 10 min gradient) to furnish 2-(3,4-dichlorophenyl)-1-[4-(isoxazol-3-ylmethyl)-8-pyrrolidin-1-yl-3,4a,5,7,8,8a-hexahydro-2H-pyrano[3,4-b]pyrazin-1-yl]ethanone (rac-5A86). ¹H-NMR (400 MHz, methanol-d₄) δ 8.84 (d, J=1.59 Hz, 1H), 7.54 (d, J=1.96 Hz, 1H), 7.48 (d, J=8.31 Hz, 1H), 7.27 (dd, J=8.31, 2.08 Hz, 1H), 6.75 (d, J=1.59 Hz, 1H), 4.79-4.81 (m, 1H), 4.50-4.63 (m, 2H), 4.18-4.45 (m, 4H), 3.59-3.94 (m, 8H), 3.35-3.39 (m, 3H) 3.10-3.22 (m, 1H), 1.93-2.14 (m, 4H).

### Step 2

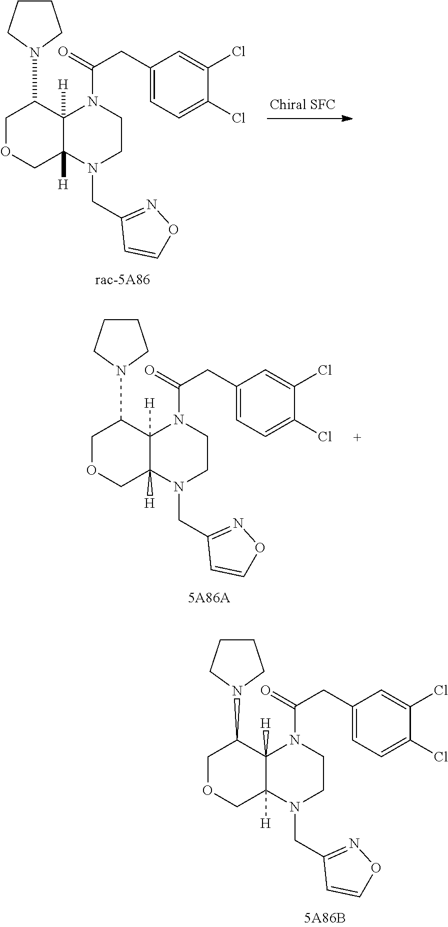 [SMILES: O=C(Cc1ccc(Cl)c(Cl)c1)N1CCN(Cc2ccon2)[C@@H]2COC[C@H](N3CCCC3)[C@H]21 ; O=C(Cc1ccc(Cl)c(Cl)c1)N1CCN(Cc2ccon2)[C@@H]2COC[C@H](N3CCCC3)[C@H]21 ; O=C(Cc1ccc(Cl)c(Cl)c1)N1CCN(Cc2ccon2)[C@H]2COC[C@@H](N3CCCC3)[C@@H]21]

Rac-5A86 was separated by Chiral SFC (column: DAICEL CHIRALPAK AD-H (250 mm×30 mm×5 μm); mobile phase: [0.1% NH₃H₂O IPA]; B%: 44%, 12 minute) to furnish 5A86A and 5A86B: (Retention time: 5A86A: 2.41 min; 5A86B: 4.40 min). LCMS m/z: 479.2 [MH³⁰ ].

The examples in Table A were prepared in a similar fashion to that shown for rac-5A86 from Intermediate A in Scheme A using the appropriate conditions.

TABLE A

| | | | 1H NMR |
|---|---|---|---|
| Example | Conditions | LCMS | (400 MHz, methanol-d4) |
| 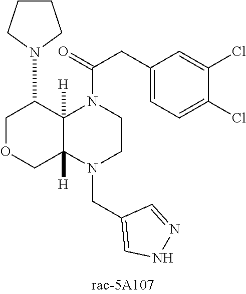 | 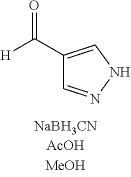 | 478.2 (MH⁺) | δ = 7.67 (s, 2H), 7.49-7.44 (m, 2H), 7.18 (dd, J = 2.1, 8.3 Hz, 1H), 4.83-4.75 (m, 2H), 4.59 (dd, J = 5.3, 11.2 Hz, 1H), 4.35 (dd, J = 4.5, 11.3 Hz, 1H), 4.21-4.12 (m, 2H), 4.06 (br t, J = 10.5 Hz, 1H), 3.93-3.80 (m, 4H), 3.70-3.57 (m, 5H), 3.29-3.23 (m, 1H), 3.20- 3.14 (m, 1H), 3.20-3.14 (m, 1H), 3.19-3.12 (m, 1H), 2.76- 2.68 (m, 1H), 2.04 (br s, 4H) |
| 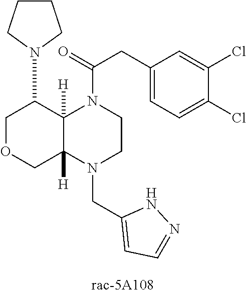 | 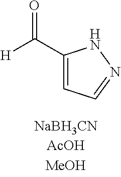 | 478.2 (MH⁺) | δ = 7.62 (d, J = 2.2 Hz, 1H), 7.47- 7.44 (m, 2H), 7.18 (dd, J = 2.0, 8.3 Hz, 1H), 6.15 (d, J = 2.2 Hz, 1H), 4.81-4.74 (m, 1H), 4.56 (dd, J = 4.9, 11.2 Hz, 1H), 4.30 (dd, J = 4.3, 11.3 Hz, 1H), 4.11-4.05 (m, 1H), 3.89-3.80 (m, 2H), 3.75 (d, J = 16.0 Hz, 2H), 3.70-3.63 (m, 2H), 3.57-3.53 (m, 1H), 3.52- 3.41 (m, 3H), 3.37 (br d, J = 10.8 Hz, 2H), 2.86 (br d, J = 11.9 Hz, 1H), 2.77 (dt, J = 5.3, 9.8 Hz, 1H), 2.34 (dt, J = 3.1, 11.5 Hz, 1H), 2.05 (br s, 4H) |
| 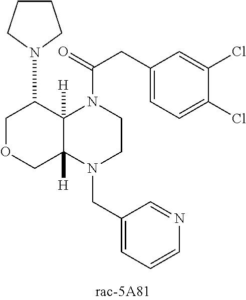 | 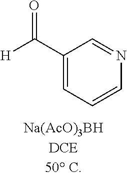 | 489.1 (MH⁺) | δ = 8.73 (br s, 2H), 8.33 (br d, J = 8.1 Hz, 1H), 7.89 (br s, 1H), 7.52- 7.45 (m, 2H), 7.23 (dd, J = 2.0, 8.2 Hz, 1H), 4.84-4.78 (m, 1H), 4.32 (td, J = 2.4, 11.2 Hz, 2H), 4.09- 4.01 (m, 2H), 3.84 (s, 2H), 3.79- 3.72 (m, 1H), 3.64 (br s, 1H), 3.57 (br t, J = 10.8 Hz, 2H), 3.52-3.44 (m, 2H), 3.36-3.32 (m, 2H), 2.90 (dt, J = 4.8, 9.8 Hz, 1H), 2.76- 2.68 (m, 1H), 2.31 (dt, J = 3.5, 11.2 Hz, 1H), 2.05 (br s, 4H) |
| 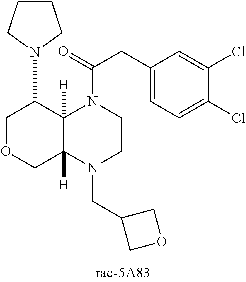 | 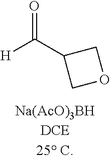 | 468.3 (MH⁺) | δ = 7.55-7.45 (m, 2H), 7.24 (dd, J = 1.9, 8.3 Hz, 1H), 4.80-4.68 (m, 3H), 4.42-4.35 (m, 3H), 4.31 (dd, J = 4.5, 11.4 Hz, 1H), 4.08 (td, J = 3.9, 15.1 Hz, 1H), 3.92 (br t, J = 10.5 Hz, 1H), 3.86 (s, 2H), 3.65-3.53 (m, 4H), 3.45 (br t, J = 10.8 Hz, 2H), 3.29-3.25 (m, 1H), 3.24-3.17 (m, 1H), 3.14-3.03 (m, 2H), 2.82 (dd, J = 6.5, 12.5 Hz, 1H), 2.52-2.44 (m, 1H), 2.03 (br s, 4H) |
| 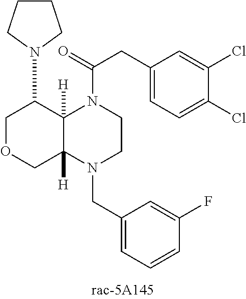 | 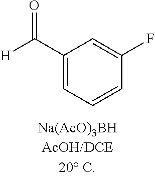 | 506.1 (MH⁺) | δ 7.44-7.45 (m, 2H), 7.41-7.43 (m, 1H), 7.21-7.22 (m, 1H), 7.06-7.19 (m, 2H), 6.96-7.08 (m, 1H), 4.28- 4.30 (m, 2H), 4.06-4.09 (m, 1H), 3.83-3.90 (m, 3H), 3.59-3.61 (m, 1H), 3.15-3.28 (m, 5H), 2.71-2.75 (m, 6H), 2.15-2.17 (m, 1H), 1.70 (s, 4H). |
| 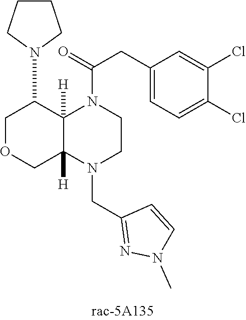 | 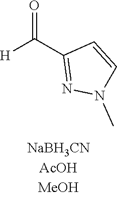 | 492.1 (MH⁺) | δ = 7.59 (s, 1H), 7.46 (dd, J = 3.1, 5.1 Hz, 2H), 7.20 (dd, J = 1.9, 8.3 Hz, 1H), 6.21 (d, J = 1.7 Hz, 1H), 4.81-4.74 (m, 1H), 4.64 (dd, J = 5.1, 11.1 Hz, 1H), 4.33 (dd, J = 4.5, 11.4 Hz, 1H), 4.19-4.11 (m, 1H), 4.11-3.91 (m, 4H), 3.91- 3.84 (m, 4H), 3.81-3.74 (m, 1H), 3.69-3.46 (m, 6H), 3.21-3.09 (m, 2H), 2.72-2.62 (m, 1H), 2.02 (br d, J = 8.4 Hz, 4H) |
| 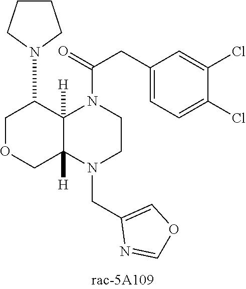 | 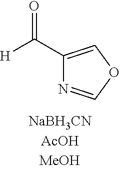 | 481.2 (MH⁺) | δ = 8.16 (s, 1H), 7.79 (s, 1H), 7.47- 7.43 (m, 2H), 7.18 (dd, J = 2.0, 8.3 Hz, 1H), 4.78 (td, J = 5.3, 10.8 Hz, 1H), 4.57 (dd, J = 5.1, 11.2 Hz, 1H), 4.30 (dd, J = 4.3, 11.2 Hz, 1H), 4.06 (td, J = 3.2, 14.6 Hz, 1H), 3.89-3.82 (m, 1H), 3.75 (d, J = 15.8 Hz, 1H), 3.71-3.58 (m, 4H), 3.57-3.43 (m, 3H), 3.43- 3.33 (m, 3H), 2.86-2.77 (m, 2H), 2.33 (dt, J = 3.5, 11.5 Hz, 1H), 2.05 (br s, 4H) |
| 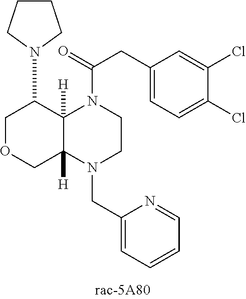 | 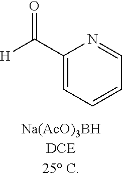 | 489.1 (MH⁺) | δ = 8.68 (br d, J = 5.0 Hz, 1H), 8.25 (t, J = 7.6 Hz, 1H), 7.79- 7.69 (m, 2H), 7.51-7.45 (m, 2H), 7.23 (dd, J = 1.9, 8.3 Hz, 1H), 4.83-4.79 (m, 2H), 4.36-4.23 (m, 3H), 4.20-4.02 (m, 3H), 3.84 (d, J = 5.0 Hz, 2H), 3.82-3.73 (m, 2H), 3.73-3.61 (m, 2H), 3.56 (br t, J = 10.8 Hz, 3H), 2.99 (dt, J = 4.9, 9.7 Hz, 1H), 2.79 (br d, J = 11.6 Hz, 1H), 2.52-2.41 (m, 1H), 2.06 (br s, 4H) |
| 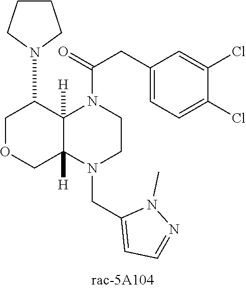 | 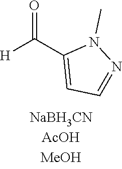 | 492.2 (MH⁺) | δ = 7.52-7.45 (m, 2H), 7.39 (d, J = 1.6 Hz, 1H), 7.22 (dd, J = 1.8, 8.3 Hz, 1H), 6.18 (d, J = 1.5 Hz, 1H), 4.79 (br d, J = 4.4 Hz, 1H), 4.45 (dd, J = 5.0, 11.1 Hz, 1H), 4.32 (dd, J = 4.5, 11.2 Hz, 1H), 4.08-4.00 (m, 1H), 3.96 (d, J = 14.2 Hz, 1H), 3.88-3.78 (m, 6H), 3.74-3.67 (m, 2H), 3.65 (br d, J = 6.4 Hz, 1H), 3.57 (br t, J = 10.7 Hz, 2H), 3.53-3.47 (m, 1H), 3.46- 3.36 (m, 4H), 2.82 (dt, J = 5.0, 9.9 Hz, 1H), 2.73-2.67 (m, 1H), 2.16 (dt, J = 3.5, 11.2 Hz, 1H), 2.05 (br s, 4H) |
| 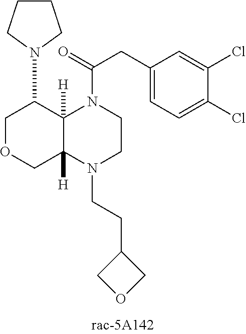 | 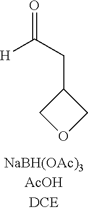 | 482.2 (MH⁺) | δ 7.42-7.56 (m, 2H), 7.18-7.31 (m, 1H), 4.26-4.49 (m, 2H), 3.79-4.22 (m, 8H), 3.51-3.77 (m, 7H), 3.35- 3.49 (m, 1H), 2.82 (d, J = 4.52 Hz, 2H), 2.60-2.77 (m, 3H), 1.87-2.52 (m, 3H), 1.67 (d, J = 3.42 Hz, 4H). |
| 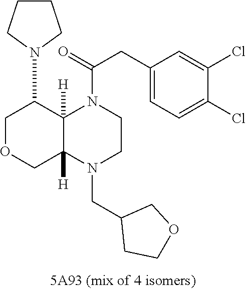 | 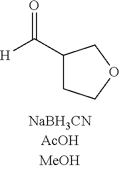 | 482.2 (MH⁺) | δ = 7.54-7.45 (m, 2H), 7.30- 7.22 (m, 1H), 4.19 (dt, J = 4.6, 10.8 Hz, 2H), 4.11-4.04 (m, 1H), 4.01-3.85 (m, 2H), 3.85-3.76 (m, 2H), 3.76-3.64 (m, 2H), 3.50 (br dd, J = 5.9, 8.3 Hz, 1H), 3.39 (br dd, J = 5.6, 8.4 Hz, 1H), 3.27 (br dd, J = 4.8, 10.3 Hz, 2H), 3.15- 2.97 (m, 2H), 2.72 (br s, 4H), 2.68-2.59 (m, 1H), 2.57-2.48 (m, 1H), 2.47-2.34 (m, 1H), 2.28- 2.12 (m, 2H), 2.07-1.90 (m, 1H), 1.71 (br s, 4H), 1.68-1.59 (m, 1H), 1.55-1.45 (m, 1H) |
| 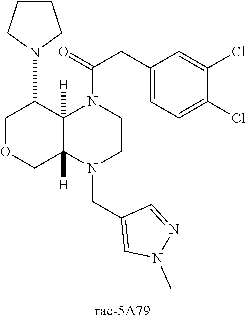 | 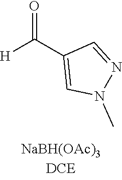 | 492.2 (MH⁺) | δ = 7.62 (s, 1H), 7.49-7.44 (m, 3H), 7.20 (dd, J = 2.0, 8.4 Hz, 1H), 4.79 (dt, J = 4.4, 10.6 Hz, 2H), 4.56 (dd, J = 5.2, 11.4 Hz, 1H), 4.34 (dd, J = 4.4, 11.5 Hz, 1H), 4.15 (td, J = 3.6, 15.1 Hz, 1H), 4.07-3.94 (m, 2H), 3.90 (s, 3H), 3.86-3.76 (m, 3H), 3.68-3.51 (m, 5H), 3.19-3.07 (m, 2H), 2.67- 2.57 (m, 1H), 2.04 (br s, 3H), 2.11-1.95 (m, 1H) |
| 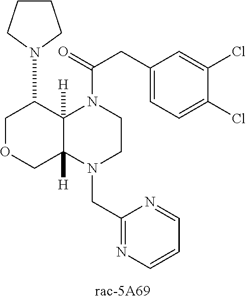 | 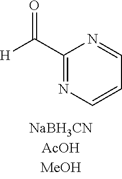 | 490.1 (MH⁺) | δ = 8.76 (d, J = 5.0 Hz, 2H), 7.48- 7.39 (m, 3H), 7.20 (dd, J = 1.8, 8.1 Hz, 1H), 4.78 (dt, J = 4.2, 10.8 Hz, 1H), 4.51 (dd, J = 5.1, 10.9 Hz, 1H), 4.30 (dd, J = 4.4, 11.4 Hz, 1H), 4.06 (br d, J = 14.5 Hz, 1H), 3.93 (s, 2H), 3.90-3.84 (m, 1H), 3.78-3.71 (m, 1H), 3.69-3.59 (m, 2H), 3.56-3.43 (m, 4H), 3.39- 3.34 (m, 2H), 3.03 (dt, J = 4.9, 9.8 Hz, 1H), 2.85 (br d, J = 12.1 Hz, 1H), 2.42 (dt, J = 3.7, 11.3 Hz, 1H), 2.12-1.92 (m, 4H) |
| 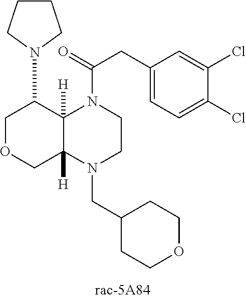 | 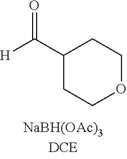 | 496.2 (MH⁺) | δ = 7.55-7.48 (m, 1H), 7.55- 7.48 (m, 1H), 7.27 (dd, J = 1.8, 8.3 Hz, 1H), 4.66 (dt, J = 4.6, 10.2 Hz, 1H), 4.38-4.29 (m, 2H), 4.15- 4.06 (m, 2H), 3.97-3.91 (m, 2H), 3.89 (d, J = 2.8 Hz, 2H), 3.77- 3.69 (m, 1H), 3.68-3.61 (m, 2H), 3.55-3.45 (m, 2H), 3.44-3.36 (m, 3H), 3.24 (br dd, J = 4.8, 10.0 Hz, 1H), 2.72 (br dd, J = 9.2, 12.6 Hz, 2H), 2.50 (br dd, J = 4.5, 13.0 Hz, 1H), 2.05 (br s, 4H), 1.90- 1.81 (m, 1H), 1.73 (br d, J = 13.4 Hz, 1H), 1.59 (br d, J = 13.2 Hz, 1H), 1.35-1.19 (m, 2H) |
| 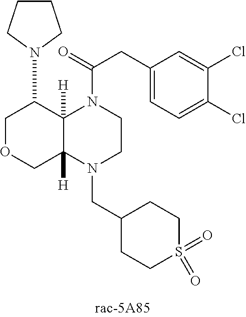 | 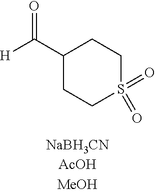 | 544.2 (MH⁺) | δ = 7.53-7.47 (m, 2H), 7.26 (dd, J = 2.0, 8.3 Hz, 1H), 4.65 (dt, J = 4.3, 10.3 Hz, 1H), 4.26 (ddd, J = 4.6, 11.1, 15.3 Hz, 2H), 4.06- 3.98 (m, 1H), 3.86 (s, 2H), 3.73 (br t, J = 10.4 Hz, 1H), 3.60-3.52 (m, 3H), 3.29-3.25 (m, 1H), 3.15- 2.94 (m, 6H), 2.77 (dt, J = 5.0, 9.9 Hz, 1H), 2.44 (br dd, J = 8.7, 13.0 Hz, 1H), 2.31-2.16 (m, 3H), 2.12 (br dd, J = 4.7, 13.0 Hz, 2H), 2.04 (br d, J = 9.7 Hz, 5H), 1.75-1.64 (m, 2H), 1.63-1.53 (m, 1H) |

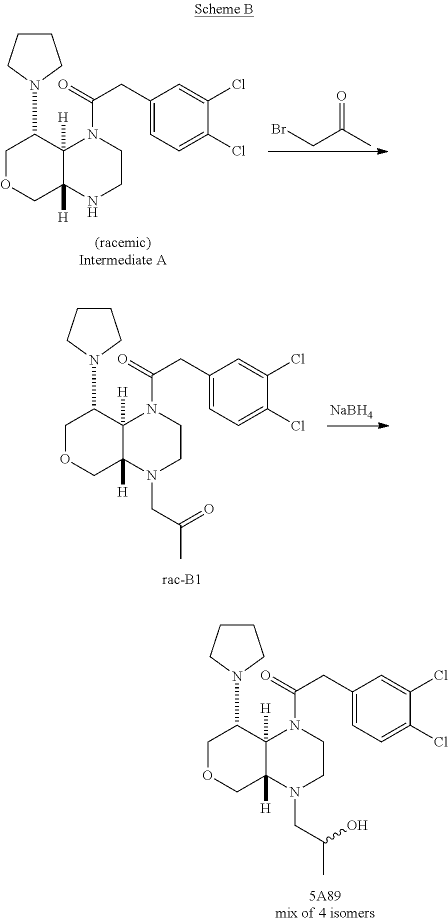

### Step 1

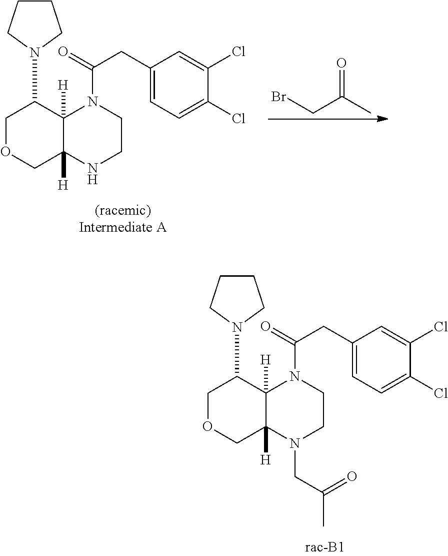

Intermediate A (100 mg, 251 □mol, 1 eq) and K2CO3 (208 mg, 1.51 mmol, 6 eq) were dissolved in DMF (15 mL). 1-Bromopropan-2-one (69 mg, 502 μmol, 2.23 μL, 2 eq) was added to the mixture. The resulting mixture was stirred at 25° C. under N₂ for 12 hrs. The reaction mixture was concentrated under reduced pressure to remove solvent. The residue was diluted with EtOAc (5 mL) and H₂O (3 mL). The mixture was extracted with EtOAc (5 mL×3). The combined organic layers were washed with brine (5 mL×3), dried over Na₂SO₄, and filtered. The filtrate was concentrated under reduced pressure to furnish rac-B1. The material was used directly in the next step without further purification.

### Step 2

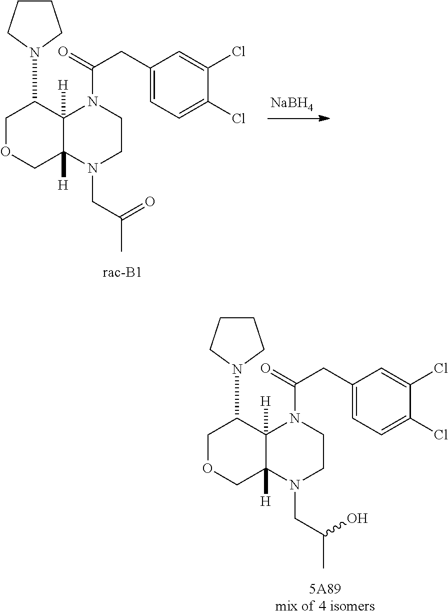

Rac-B1 (15 mg, 33 □mol, 1 eq) was dissolved in MeOH (5 mL). Sodium borohydride (1.5 mg, 40 μmol, 1.2 eq) was added at 0° C. under N₂. The resulting mixture was stirred at 25° C. under N₂ for 12 hrs. The reaction mixture was concentrated under reduced pressure to remove solvent. The residue was diluted with EtOAc (5 mL) and H₂O (3 mL). The mixture was extracted with EtOAc (5 mL×3). The combined organic layers were washed with brine (5 mL×3), dried over Na₂SO₄, and filtered. The filtrate was concentrated under reduced pressure. The residue was purified by preparative-HPLC (column: Waters Xbridge 150 mm×25 mm, 5 μ; mobile phase: [water (0.1% TFA)-ACN]; B%: 10%-35%, 13 minute gradient) to furnish 5A89 as a mixture of 4 isomers. LCMS m/z: 456.3 [MH⁺]. 1H NMR (400 MHz, methanol-d4) δ=7.53-7.45 (m, 2H), 7.27-7.21 (m, 1H), 4.81-4.65 (m, 1H), 4.48-4.40 (m, 1H), 4.33 (dd, J=4.6, 11.5 Hz, 1H), 4.29-4.14 (m, 2H), 4.14-3.91 (m, 2H), 3.91-3.86 (m, 2H), 3.86-3.76 (m, 1H), 3.75-3.52 (m, 6H), 3.28-3.16 (m, 1H), 3.06-2.93 (m, 2H), 2.02 (br s, 4H), 1.23-1.15 (m, 3H).

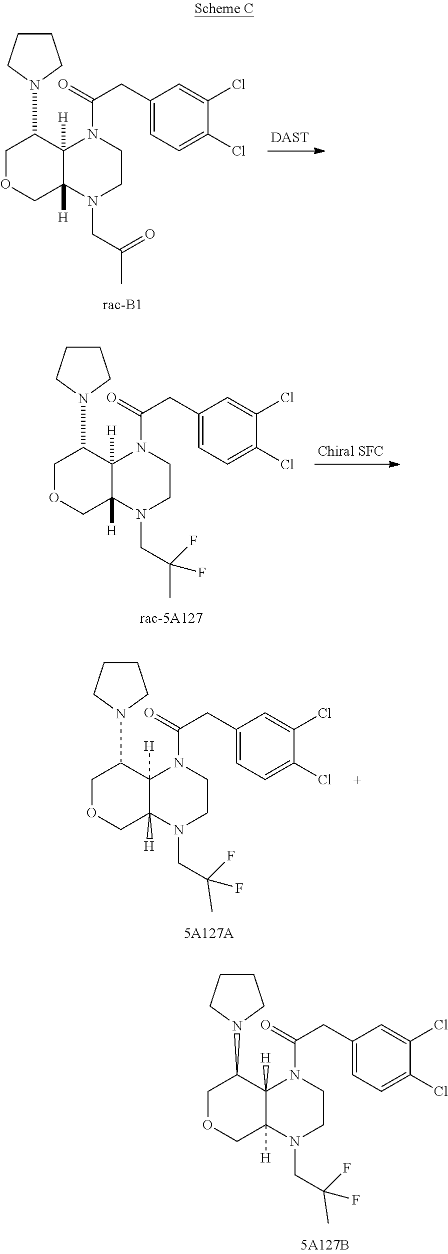

### Step 1

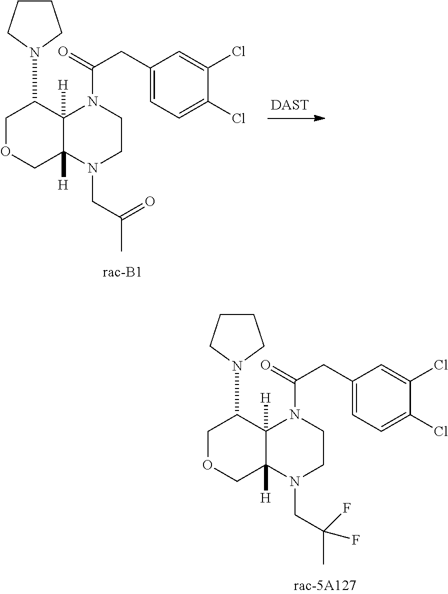

To a solution of DAST (2.31 g, 14.30 mmol, 1.89 mL, 50 eq) in DCM (5 mL) was added dropwise 1-[1-[2-(3,4-dichlorophenyl)acetyl]-8-pyrrolidin-1-yl-3,4a,5,7,8,8a-hexahydro-2H-pyrano[3,4-b]pyrazin-4-yl]propan-2-one (rac-B1: 130 mg, 286 μmol, 1 eq) at −78° C. over 30 min. The resulting mixture was stirred at 25° C. for 11.5 hr. The reaction mixture was diluted with saturated aqueous NaHCO₃ (30 mL). The mixture was extracted with ethyl acetate (30 mL×3). The combined organic layers were dried over Na₂SO₄ and filtered. The filtrate was concentrated under reduced pressure. The residue was purified by preparative-HPLC (column: Waters Xbridge 150 mm×25 mm, 5μ; mobile phase: [water(10 mM NH₄HCO₃)-ACN]; B%: 40%-70%, 10 minute gradient) to furnish rac-5A127. LCMS m/z: 476.3 [MH³⁰ ]. ¹H NMR (400 MHz, methanol-d4, TFA salt) δ=7.52-7.46 (m, 2H), 7.24 (dd, J=1.9, 8.3 Hz, 1H), 4.59 (br s, 1H), 4.28-4.22 (m, 1H), 4.18 (br dd, J=4.7, 11.2 Hz, 1H), 3.99 (br d, J=14.5 Hz, 1H), 3.88-3.83 (m, 3H), 3.60 (br s, 1H), 3.54-3.42 (m, 3H), 3.26 (br d, J=11.2 Hz, 1H), 3.23-3.19 (m, 2H), 3.03 (br dd, J=3.5, 12.0 Hz, 1H), 2.93-2.84 (m, 2H), 2.70-2.56 (m, 2H), 1.97 (br s, 4H), 1.55 (t, J=18.8 Hz, 3H).

### Step 2

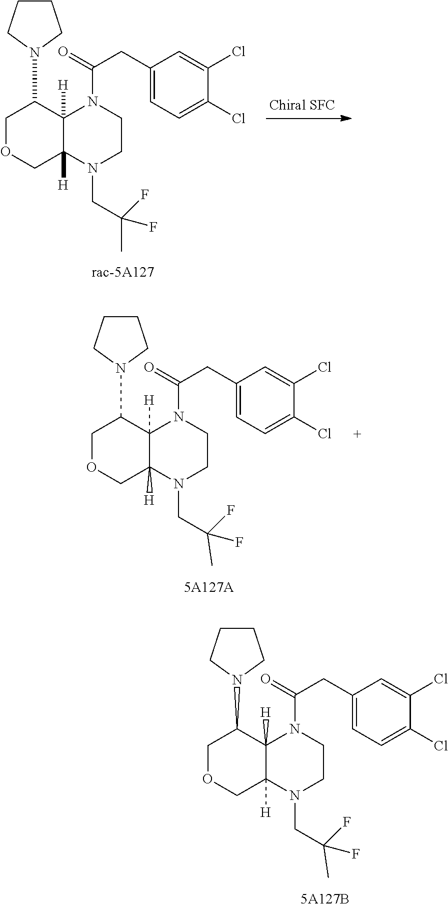

2-(3,4-Dichlorophenyl)-1-[4-(2,2-difluoropropyl)-8-pyrrolidin-1-yl-3,4a,5,7,8,8a-hexahydro-2H-pyrano[3,4-b]pyrazin-1-yl]ethanone (rac-5A127) was separated by SFC (column: DAICEL CHIRALPAK IC (250 mm×30 mm, 10 μm); mobile phase: [0.1% NH₃H₂O MeOH]; B%: 45%-45%, 7 minutes) to furnish 5A127A and 5A127B. (Retention time: 5A127A: 1.82 min; 5A127B: 2.16 min). LCMS m/z: 476.1 [MH⁺].

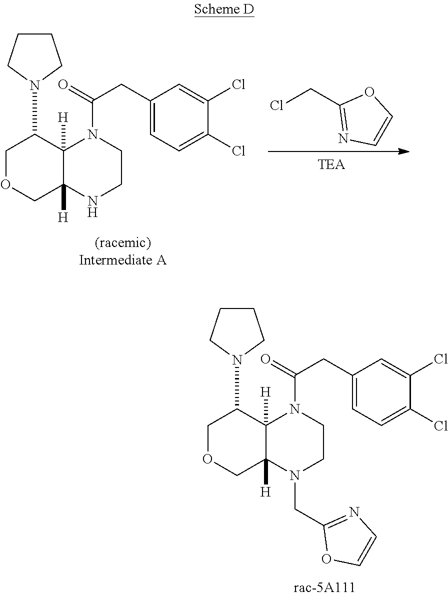 [SMILES: O=C(Cc1ccc(Cl)c(Cl)c1)N1CCN[C@@H]2COC[C@H](N3CCCC3)[C@H]21 ; ClCc1ncco1 ; O=C(Cc1ccc(Cl)c(Cl)c1)N1CCN(Cc2ncco2)[C@@H]2COC[C@H](N3CCCC3)[C@H]21]

Intermediate A (10 mg, 25 μmol, 1 eq) and 2-(chloromethyl)oxazole (5.9 mg, 50 μmol, 2.23 μL, 2 eq) were dissolved in DMF (2 mL),. Triethylamine (13 mg, 126 μmol, 17 μL, 5 eq) was added. The resulting mixture was stirred at 40° C. under N₂ for 12 hrs. The reaction mixture was concentrated under reduced pressure to remove solvent. The residue was diluted with EtOAc (5 mL) and H₂O (3 mL). The mixture was extracted with EtOAc (5 mL×3). The combined organic layers were washed with brine (5 mL×3), dried over Na₂SO₄, and filtered. The filtrate was concentrated under reduced pressure. The residue was purified by preparative-HPLC (column: Phenomenex Synergi C_{18 100} mm×30 mm×4 μm; mobile phase: [water (0.1% TFA)-ACN]; B%: 15%-45%, 10 minute gradient) to furnish rac-5A111. LCMS m/z: 479.2 [MH⁺]. 1H NMR (400 MHz, methanol-d4) δ=7.89 (d, J=0.9 Hz, 1H), 7.51-7.47 (m, 2H), 7.22 (dd, J=2.0, 8.3 Hz, 1H), 7.17 (s, 1H), 4.85-4.80 (m, 1H), 4.50 (dd, J=5.0, 11.2 Hz, 1H), 4.33 (dd, J=4.4, 11.1 Hz, 1H), 4.10 (td, J=3.0, 14.5 Hz, 1H), 3.88-3.83 (m, 2H), 3.92-3.83 (m, 1H), 3.81-3.74 (m, 1H), 3.63-3.55 (m, 2H), 3.54-3.45 (m, 3H), 3.39-3.35 (m, 3H), 2.83 (td, J=2.9, 11.7 Hz, 1H), 2.75 (dt, J=5.1, 9.9 Hz, 1H), 2.32 (dt, J=3.5, 11.5 Hz, 1H), 2.11-1.98 (m, 4H).

The examples in Table B were prepared in a similar fashion to that shown for rac-5A111 from Intermediate A in Scheme D using the appropriate conditions.

TABLE B

| Example | Conditions | LCMS | 1H NMR (400 MHz) |
|---|---|---|---|
| 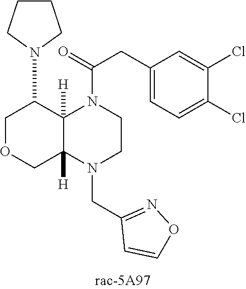 | 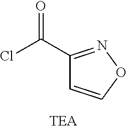 | 493.2 (MH⁺) | (methanol-d4) δ = 8.79 (d, J = 1.3 Hz, 1H), 7.42 (d, J = 1.8 Hz, 1H), 7.34 (d, J = 8.2 Hz, 1H), 7.14 (dd, J = 1.7, 8.3 Hz, 1H), 6.62 (d, J = 1.5 Hz, 1H), 4.40 (br d, J = 10.6 Hz, 3H), 4.21 (br s, 3H), 4.04- 3.86 (m, 3H), 3.86-3.73 (m, 3H), 3.68 (br d, J = 9.9 Hz, 4H), 3.37 (br s, 2H), 2.07 (br s, 2H), 2.02 (br s, 2H) |
| 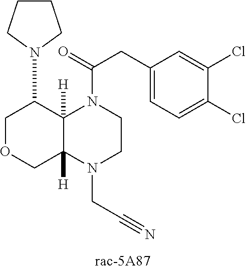 | 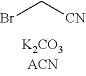 | 437.1 (MH⁺) | (methanol-d4) δ = 7.48-7.43 (m, 2H), 7.23 (dd, J = 2.0, 8.2 Hz, 1H), 4.38 (br s, 1H), 4.16 (dd, J = 4.9, 11.2 Hz, 1H), 4.12-3.99 (m, 2H), 3.89 (d, J = 15.7 Hz, 1H), 3.79- 3.66 (m, 2H), 3.60-3.53 (m, 1H), 3.41-3.33 (m, 1H), 3.27-3.15 (m, 2H), 3.09 (br t, J = 10.7 Hz, 1H), 2.86-2.77 (m, 2H), 2.75 (br dd, J = 5.0, 9.8 Hz, 4H), 2.49 (dt, J = 3.3, 11.4 Hz, 1H), 1.71 (br s, 4H) |
| 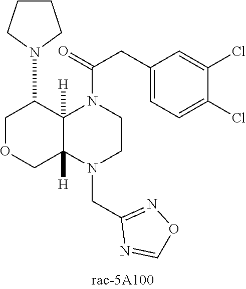 | 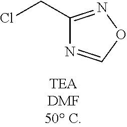 | 480.3 (MH⁺) | (methanol-d4) δ = 9.23 (s, 1H), 7.52-7.45 (m, 2H), 7.20 (dd, J = 1.8, 8.4 Hz, 1H), 4.81-4.76 (m, 1H), 4.50 (dd, J = 5.1, 11.0 Hz, 1H), 4.35-4.28 (m, 1H), 4.11- 4.03 (m, 1H), 3.98-3.84 (m, 3H), 3.80-3.74 (m, 1H), 3.66-3.58 (m, 2H), 3.57-3.49 (m, 2H), 3.49- 3.40 (m, 2H), 3.40-3.34 (m, 2H), 2.88-2.78 (m, 2H), 2.46-2.36 (m, 1H), 2.12-1.93 (m, 4H) |
| 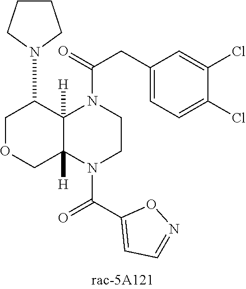 | 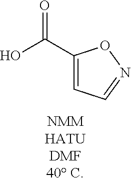 | 493.2 (MH⁺) | (methanol-d4) δ = 8.50 (d, J = 1.8 Hz, 1H), 7.42 (d, J = 2.0 Hz, 1H), 7.31 (d, J = 8.2 Hz, 1H), 7.13 (dd, J = 2.0, 8.3 Hz, 1H), 6.77 (d, J = 2.0 Hz, 1H), 4.53 (br s, 1H), 4.39 (dd, J = 4.1, 11.2 Hz, 1H), 4.26 (br s, 1H), 4.23-4.16 (m, 1H), 4.14- 4.01 (m, 1H), 4.14-4.01 (m, 1H), 4.01-3.89 (m, 3H), 3.81 (br d, J = 2.1 Hz, 2H), 3.78-3.69 (m, 2H), 3.68 (br s, 1H), 3.53-3.44 (m, 1H), 3.44-3.34 (m, 2H), 2.13- 1.93 (m, 4H) |
| 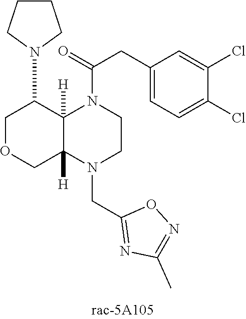 | 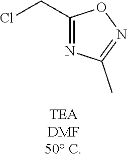 | 494.2 (MH⁺) | (methanol-d4) δ = 7.49-7.45 (m, 2H), 7.21 (dd, J = 2.0, 8.3 Hz, 1H), 4.82-4.78 (m, 1H), 4.39 (dd, J = 5.0, 11.1 Hz, 1H), 4.31 (dd, J = 4.4, 11.2 Hz, 1H), 4.09 (td, J = 3.1, 14.5 Hz, 1H), 3.98 (d, J = 1.6 Hz, 2H), 3.90-3.84 (m, 1H), 3.80- 3.74 (m, 1H), 3.67-3.54 (m, 3H), 3.54-3.43 (m, 3H), 3.39-3.33 (m, 2H), 2.94-2.85 (m, 2H), 2.46 (dt, J = 3.5, 11.3 Hz, 1H), 2.37 (s, 3H), 2.10-1.96 (m, 4H) |
| 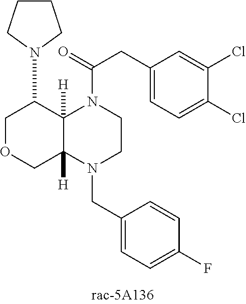 | 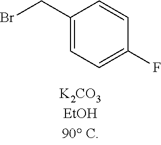 | 506.2 (MH⁺) | (methanol-d4) δ 7.40-7.47 (m, 2H), 7.15-7.32 (m, 3H), 6.97-7.06 (m, 2H), 4.18-4.45 (m, 2H), 4.07 (d, J = 11.00, 4.77 Hz, 1H), 3.77-3.95 (m, 3H), 3.63 (d, J = 15.77 Hz, 1H), 3.11-3.29 (m, 5H), 2.61-2.81 (m, 6H), 2.11 (td, J = 11.31, 3.55 Hz, 1H), 1.57-1.81 (m, 4H). |
| 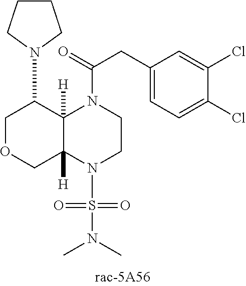 | 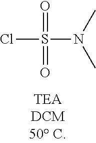 | 505.1 (MH⁺) | (methanol-d4) δ = 7.48-7.43 (m, 2H), 7.24-7.19 (m, 1H), 4.10 (br d, J = 7.9 Hz, 1H), 3.98 (br s, 2H), 3.93-3.74 (m, 4H), 3.55 (br s, 2H), 3.51-3.41 (m, 2H), 3.35 (br d, J = 1.6 Hz, 1H), 2.79 (s, 6H), 2.76-2.64 (m, 4H), 1.69 (br s, 4H) |
| 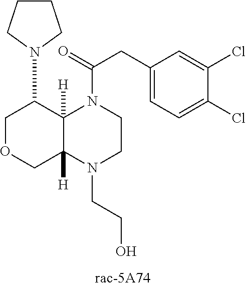 | 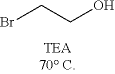 | 442.2 (MH⁺) | (methanol-d4) δ 7.44-7.48 (m, 2H), 7.23 (dd, J = 8.27, 1.87 Hz, 1H), 4.21 (dd, J = 11.03, 4.85 Hz, 2H), 4.06 (dd, J = 10.91, 4.74 Hz, 1H), 3.85-3.99 (m, 2H), 3.53-3.69 (m, 3H), 3.23 (t, J = 10.91 Hz, 3H), 3.11 (t, J = 10.80 Hz, 1H), 3.00 (d, J = 11.69 Hz, 1H), 2.62-2.80 (m, 6H), 2.31-2.44 (m, 2H), 1.69 (s, 4H). |
| 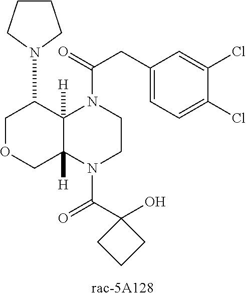 | 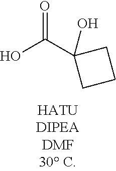 | 496.1 (MH⁺) | (methanol-d4) δ = 7.45-7.40 (m, 2H), 7.17 (dd, J = 2.0, 8.3 Hz, 1H), 4.15-3.87 (m, 5H), 3.80-3.48 (m, 5H), 3.34 (br s, 1H), 2.72 (br d, J = 5.0 Hz, 4H), 2.44 (br s, 2H), 2.20- 1.95 (m, 2H), 1.88-1.76 (m, 1H), 1.73-1.63 (m, 4H), 1.56-1.44 (m, 1H) |
| 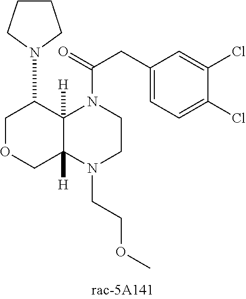 | 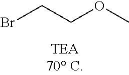 | 456.1 (MH⁺) | (methanol-d4) δ 7.42-7.52 (m, 2H), 7.24 (d, J = 8.25, 1.90 Hz, 1H), 4.14-4.37 (m, 2H), 3.62-4.13 (m, 5H), 3.36-3.58 (m, 3H), 3.02-3.29 (m, 4H), 2.88-3.01 (m, 1H), 2.62- 2.86 (m, 6H), 2.32-2.56 (m, 2H), 1.60-1.78 (m, 4H). |
| 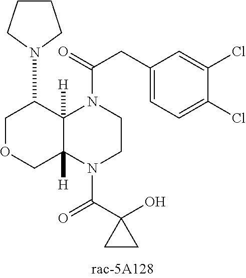 | 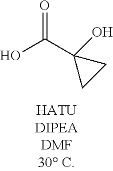 | 482.2 (MH⁺) | (DMSO-d6) δ = 7.47-7.41 (m, 2H), 7.18 (dd, J = 1.8, 8.2 Hz, 1H), 4.18-4.02 (m, 3H), 3.93 (br dd, J = 6.2, 12.2 Hz, 1H), 3.81-3.57 (m, 5H), 3.45-3.33 (m, 2H), 2.88-2.68 (m, 4H), 1.73 (br s, 4H), 1.21-1.13 (m, 2H), 0.95-0.87 (m, 1H), 0.82- 0.74 (m, 1H) |
| 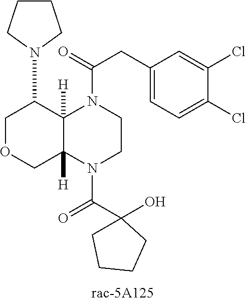 | 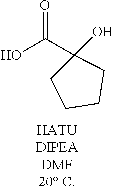 | 510.2 (MH⁺) | (methanol-d4) δ 7.45-7.47 (m, 2H), 7.20 (d, J = 6.4 Hz, 1H), 4.42-4.3 (m, 1H), 4.03- 4.14 (m, 3H), 3.69-4.03 (m, 6H), 3.36 (m, 1H), 2.75-2.76 (m, 4H), 1.96 (m, 2H), 1.61-1.77 (m, 10H) |
| 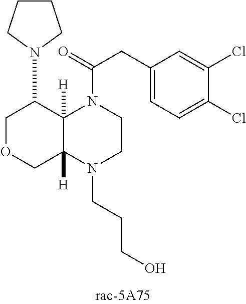 | 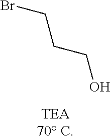 | 456.3 (MH⁺) | (methanol-d4) δ = 7.54-7.47 (m, 2H), 7.25 (dd, J = 2.0, 8.3 Hz, 1H), 4.72 (dt, J = 4.4, 10.5 Hz, 1H), 4.31 (ddd, J = 4.8, 8.4, 11.3 Hz, 2H), 4.09 (td, J = 3.8, 14.9 Hz, 1H), 3.88 (s, 2H), 3.77 (br t, J = 10.3 Hz, 1H), 3.64 (br t, J = 6.4 Hz, 1H), 3.60 (br s, 1H), 3.58-3.56 (m, 2H), 3.56-3.52 (m, 2H), 3.41- 3.33 (m, 3H), 3.15-3.08 (m, 1H), 2.98-2.89 (m, 1H), 2.84 (td, J = 7.7, 13.3 Hz, 1H), 2.56-2.43 (m, 2H), 2.10-1.96 (m, 4H), 1.73- 1.63 (m, 2H), 1.31 (tt, J = 1.7, 7.3 Hz, 3H) |
| 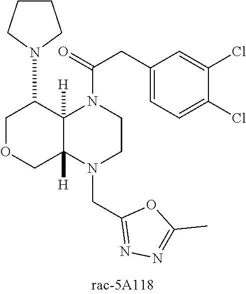 | 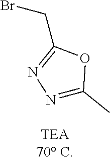 | 494.1 (MH⁺) | (methanol-d4) δ = 7.50-7.46 (m, 2H), 7.22 (dd, J = 1.8, 8.3 Hz, 1H), 4.84-4.78 (m, 1H), 4.42 (dd, J = 5.0, 11.1 Hz, 1H), 4.31 (dd, J = 4.3, 11.1 Hz, 1H), 4.10 (br d, J = 14.7 Hz, 1H), 3.95 (s, 2H), 3.90- 3.75 (m, 3H), 3.67-3.54 (m, 3H), 3.54-3.42 (m, 3H), 3.39-3.34 (m, 2H), 2.92-2.84 (m, 2H), 2.53 (s, 3H), 2.51-2.44 (m, 1H), 2.11- 1.92 (m, 5H) |
| 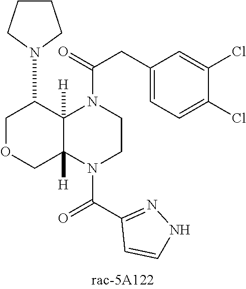 | 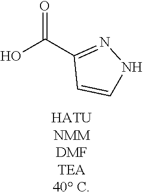 | 492.2 (MH⁺) | (methanol-d4) δ = 7.68 (d, J = 2.3 Hz, 1H), 7.38 (d, J = 2.0 Hz, 1H), 7.25 (d, J = 8.3 Hz, 1H), 7.07 (dd, J = 2.0, 8.3 Hz, 1H), 6.57 (d, J = 2.3 Hz, 1H), 4.45-4.34 (m, 4H), 4.21 (br dd, J = 3.5, 10.5 Hz, 2H), 3.96 (br dd, J = 13.0, 18.2 Hz, 2H), 3.90-3.82 (m, 1H), 3.78 (d, J = 3.1 Hz, 2H), 3.75-3.59 (m, 4H), 3.44-3.33 (m, 2H), 2.16-1.94 (m, 4H) |
| 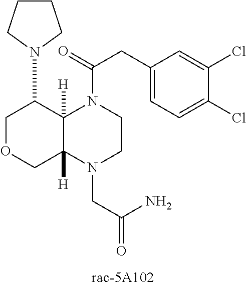 | 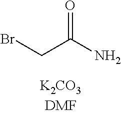 | 455.2 (MH⁺) | (methanol-d4) δ = 7.48-7.42 (m, 2H), 7.22 (dd, J = 2.0, 8.2 Hz, 1H), 4.27 (br s, 1H), 4.06 (dt, J = 4.9, 10.2 Hz, 2H), 3.95 (br d, J = 14.4 Hz, 1H), 3.89 (d, J = 15.7 Hz, 1H), 3.64 (br d, J = 15.7 Hz, 1H), 3.39 (br d, J = 12.5 Hz, 1H), 3.29-3.20 (m, 3H), 3.15 (t, J = 10.7 Hz, 1H), 2.93-2.77 (m, 3H), 2.69 (br s, 4H), 2.53-2.41 (m, 1H), 1.74- 1.66 (m, 4H) |
| 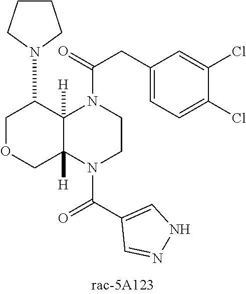 | 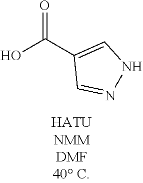 | 492.2 (MH⁺) | (methanol-d4) δ = 7.76 (s, 2H), 7.38 (d, J = 2.0 Hz, 1H), 7.25 (d, J = 8.3 Hz, 1H), 7.09 (dd, J = 2.0, 8.3 Hz, 1H), 4.67 (t, J = 10.8 Hz, 1H), 4.39 (dd, J = 4.3, 11.2 Hz, 1H), 4.32-4.25 (m, 1H), 4.17- 4.03 (m, 2H), 3.97 (dd, J = 7.3, 14.2 Hz, 1H), 3.89-3.69 (m, 7H), 3.64 (br s, 1H), 3.54-3.46 (m, 1H), 3.37 (br s, 2H), 2.16-1.89 (m, 4H) |
| 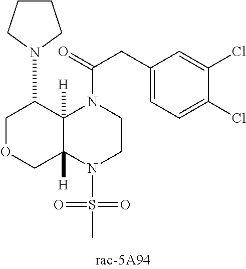 | 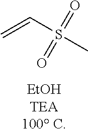 | 504.1 (MH⁺) | (methanol-d4) δ = 7.51 (d, J = 8.2 Hz, 2H), 7.26 (dd, J = 2.0, 8.3 Hz, 1H), 4.81-4.72 (m, 2H), 4.32 (td, J = 5.3, 11.1 Hz, 2H), 4.09 (td, J = 3.5, 14.7 Hz, 1H), 3.94-3.82 (m, 2H), 3.66 (br dd, J = 9.6, 11.1 Hz, 2H), 3.60-3.45 (m, 4H), 3.40- 3.35 (m, 3H), 3.25-3.12 (m, 2H), 3.08-3.05 (m, 1H), 3.04 (s, 3H), 2.86-2.75 (m, 2H), 2.38 (dt, J = 3.6, 11.1 Hz, 1H), 2.14-1.96 (m, 4H) |
| 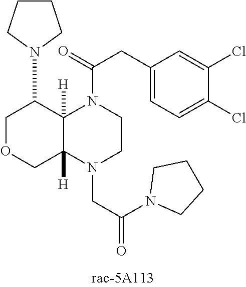 | 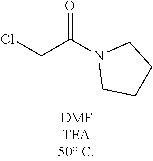 | 509.3 (MH⁺) | (methanol-d4) δ = 7.52-7.46 (m, 2H), 7.24 (dd, J = 2.1, 8.3 Hz, 1H), 4.71 (dt, J = 4.4, 10.5 Hz, 1H), 4.29 (dd, J = 4.4, 11.2 Hz, 1H), 4.23-4.18 (m, 1H), 4.07 (td, J = 3.8, 14.8 Hz, 1H), 3.86 (d, J = 2.2 Hz, 2H), 3.79-3.72 (m, 1H), 3.65- 3.51 (m, 5H), 3.46-3.40 (m, 4H), 3.40-3.36 (m, 2H), 3.34 (d, J = 5.7 Hz, 2H), 3.01 (td, J = 3.6, 11.5 Hz, 1H), 2.77 (dt, J = 3.7, 10.9 Hz, 1H), 2.04 (br s, 3H), 1.99-1.93 (m, 3H), 1.91-1.83 (m, 2H) |
| 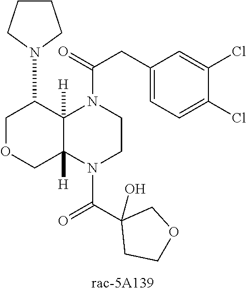 | 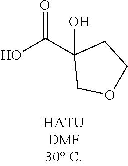 | 512.2 (MH⁺) | (methanol-d4) δ 7.42-7.52 (m, 2H), 7.21 (d, J = 8.31, 1.96 Hz, 1H), 4.44-4.63 (m, 1H), 4.02-4.42 (m, 6H), 3.53-4.00 (m, 14 H) 1.87-2.32 (m, 6H). |
| 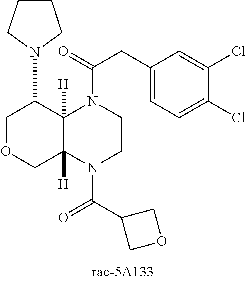 | 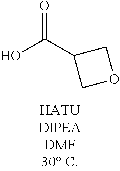 | 512.2 (MH⁺) | (CDCl₃) δ 7.41-7.27 (m, 4H), 7.08- 7.07 (m, 1H), 4.92-4.62 (m, 6H), 4.40-3.51 (m, 8H), 3.50-2.50 (m, 7H), 1.80-1.59 (m, 6H) |
| 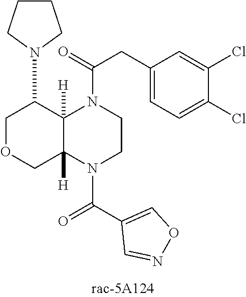 | 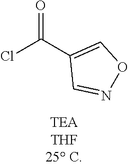 | 493.2 (MH⁺) | (DMSO-d6) δ 9.04 (s, 2H), 7.52 (d, J = 8.2 Hz, 1H), 7.48 (s, 1H), 7.18- 7.12 (m, 1H), 4.55 (br s, 1H), 4.32- 4.08 (m, 2H), 3.93 (br d, J = 7.1 Hz, 1H), 3.82 (s, 3H), 3.73-3.51 (m, 5H), 3.48-3.40 (m, 2H), 3.17 (br s, 1H), 3.27-3.05 (m, 1H), 1.97- 1.66 (m, 4H) |

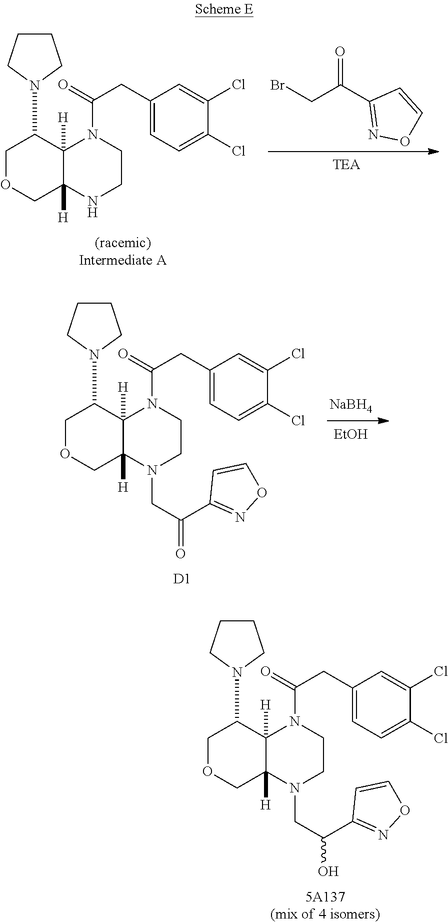

### Step 1

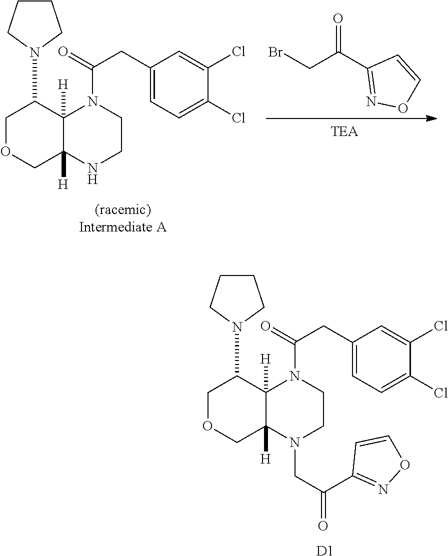

To a solution of 2-(3,4-dichlorophenyl)-1-(8-pyrrolidin-1-yl-2,3,4,4a,5,7,8,8a-octahydropyrano[3,4-b]pyrazin-1-yl)ethanone (700 mg, 1.76 mmol, 1 eq) in THF (25 mL) was added Et₃N (533 mg, 5.27 mmol, 734 μL, 3 eq) and 2-bromo-1-isoxazol-3-yl-ethanone (954 mg, 3.51 mmol, 2 eq). The mixture was stirred at 20° C. for 12 hr under N₂. The reaction mixture was quenched by addition H₂O (20 mL). The mixture was extracted with DCM (20 mL×3). The combined organic layers were dried over Na₂SO₄, filtered and concentrated. The residue was dissolved CH₃CN (4 mL). Then the solution was added 1N HCl_{(aq.)} (0.5 mL). The solution was purified by preparative-HPLC (column: Phenomenex Luna C_{18 200} mm×40 mm×10 μm; mobile phase: [water(0.05% HCl)-ACN]; B%: 20%-40%, 10 minute gradient) to furnish 2-(3,4-dichlorophenyl)-1-[4-(2-isoxazol-3-yl-2-oxo-ethyl)-8-pyrrolidin-1-yl-3,4a,5,7,8,8a-hexahydro-2H-pyrano[3,4-b]pyrazin-1-yl]ethenone.

### Step 2

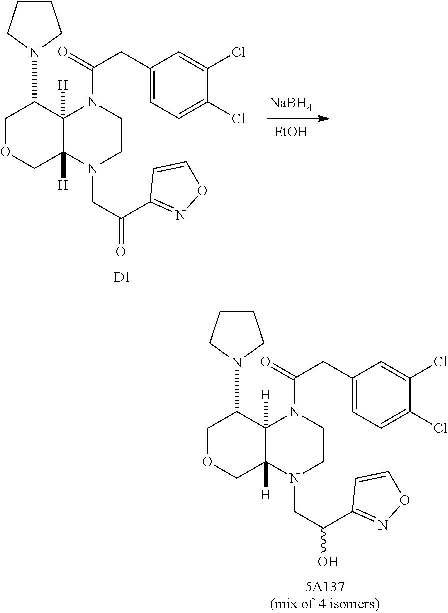

To a solution of 2-(3,4-dichlorophenyl)-1-[4-(2-isoxazol-3-yl-2-oxo-ethyl)-8-pyrrolidin-1-yl-3,4a,5,7,8,8a-hexahydro-2H-pyrano[3,4-b]pyrazin-1-yl]ethanone (120 mg, 237 μmol, 1 eq) in EtOH (4 mL) was added NaBH₄ (45 mg, 1.2 mmol, 5 eq). The mixture was stirred at 20° C. for 1 hr. The reaction mixture was quenched by addition H₂O (15 mL). The resulting mixture was extracted with DCM (15 mL×3). The combined organic layers were dried over Na₂SO₄, filtered, and concentrated. The residue was purified by preparative-HPLC (column: Luna C_{18 100} mm×30 mm×5μ; mobile phase: [water(0.04% HCl)-ACN]; B%: 10%-40%, 10 minute gradient) to furnish 2-(3,4-dichlorophenyl)-1-[4-(2-hydroxy-2-isoxazol-3-yl-ethyl)-8-pyrrolidin-1-yl-3,4a,5,7,8,8a-hexahydro-2H-pyrano[3,4-b]pyrazin-1-yflethenone (5A137). LCMS m/z: 509.1 [MH⁺]. 1H NMR (400 MHz, methanol-d4) δ=8.67-8.71 (m, 1H), 7.54 (d, J=1.76 Hz, 1H), 7.49 (d, J=8.38 Hz, 1H), 7.27 (d, J=8.16 Hz, 1H), 6.60-6.67 (m, 1H), 5.33 (dd, J=10.25, 3.42 Hz, 1H), 4.69-4.83 (m, 1H), 4.53 (dd, J=11.14, 5.18 Hz, 1H), 4.26-4.44 (m, 3H), 3.82-4.04 (m, 5H), 3.44-3.80 (m, 7H), 3.31-3.36(m, 1H), 3.18-3.28 (m, 1H), 1.92-2.14 (m, 4H).

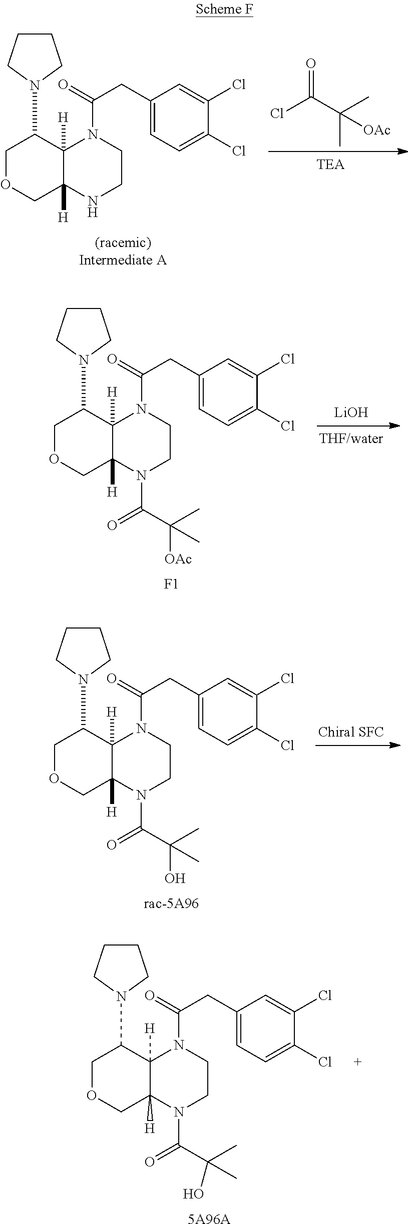

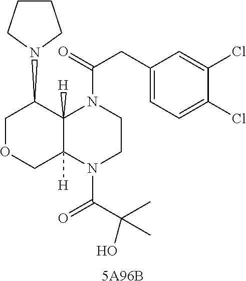

### Step 1

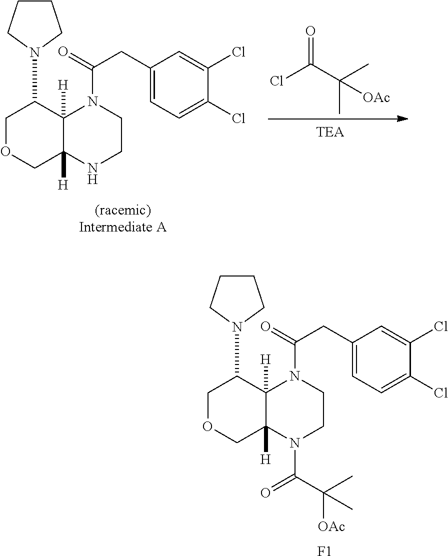

To a solution of Int.A (20 mg, 50 μmol, 1 eq) in DCM (2 mL) was added TEA (25 mg, 251 μmol, 35 μL, 5 eq) and 1-chloro-2-methyl-1-oxopropan-2-yl acetate (25 mg, 151 μmol, 22 μL, 3 eq). The mixture was stirred at 25° C. for 3 hours under a N₂ atmosphere. The reaction mixture was concentrated under reduced pressure to remove solvent. The residue was diluted with EtOAc (5 mL) and H₂O (3 mL),. The mixture was extracted with EtOAc (5 mL×3). The combined organic layers were washed with brine (5 mL×3), dried over Na₂SO₄, and filtered. The filtrate was concentrated under reduced pressure to furnish F1. The material was used directly in the next step without further purification.

### Step 2

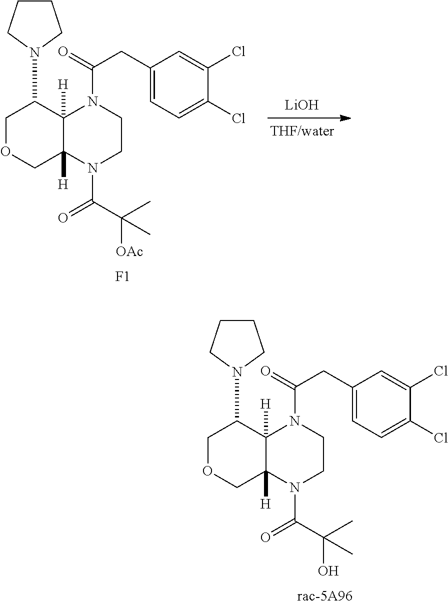

The acetate F1 (25 mg, 48 μmol, 1 eq) was dissolved in THF (2 mL) and H₂O (0.4 mL). Lithium hydroxide (1.36 mg, 57 μmol, 1.2 eq) was added. The resulting mixture was stirred at 50° C. under N₂ for 12 hrs. The reaction mixture was concentrated under reduced pressure to remove solvent. The residue was diluted with EtOAc (5 mL) and H₂O (3 mL). The mixture was extracted with EtOAc (5 mL×3). The combined organic layers were washed with brine (5 mL×3), dried over Na₂SO₄, and filtered. The filtrate was concentrated under reduced pressure. The residue was purified by preparative-HPLC (column: Waters Xbridge 150 mm×25 mm×5μ; mobile phase: [water (10 mM NH₄HCO₃)-ACN]; B%: 35%-65%, 10 minute gradient) to furnish 1-((4aS,8R,8aR)-1-(2-(3,4-dichlorophenyl)acetyl)-8-(pyrrolidin-1-yl)octahydro-4H-pyrano[3,4-b]pyrazin-4-yl)-2-hydroxy-2-methylpropan-1-one (rac-5A96). LCMS m/z: 484.0 [MH⁺]. 1H NMR (400 MHz, methanol-d4) δ=7.46-7.40 (m, 2H), 7.18 (dd, J=1.7, 8.2 Hz, 1H), 4.56 (s, 1H), 4.14 (br d, J=8.3 Hz, 3H), 4.09-3.89 (m, 3H), 3.88-3.72 (m, 3H), 3.72-3.53 (m, 2H), 3.48 (br s, 1H), 3.38 (br s, 2H), 2.84 (br s, 4H), 1.74 (br s, 4H), 1.33 (br s, 6H).

### Step 3

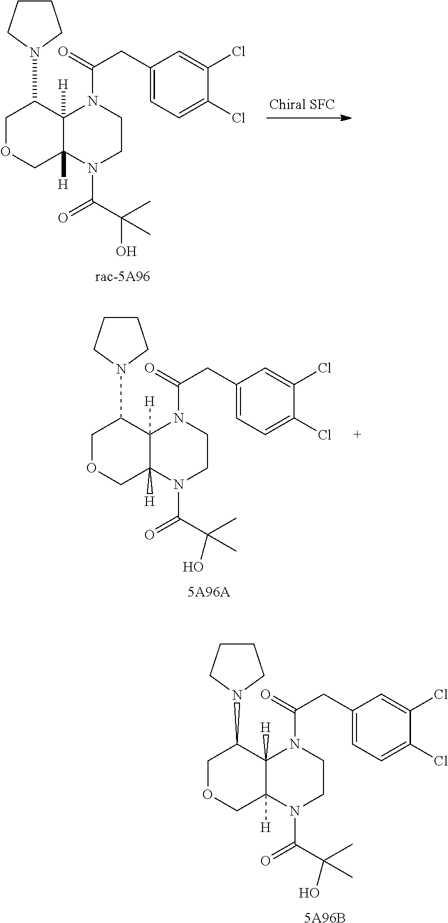

1-((4aS,8R,8aR)-1-(2-(3,4-dichlorophenyl)acetyl)-8-(pyrrolidin-1-yl)octahydro-4H-pyrano[3,4-b]pyrazin-4-yl)-2-hydroxy-2-methylpropan-1-one (rac-5A96) was separated by SFC (SFC80 preparative column: Chiralpak AS, 250 mm×25 mm×10μ Mobile phase: A:CO2 and B: EtOH (0.1% NH₃H₂O); B%=35%) to furnish 5A96A and 5A96B. (Retention time: 5A96A: 1.18 min; 5A96B: 1.49 min).

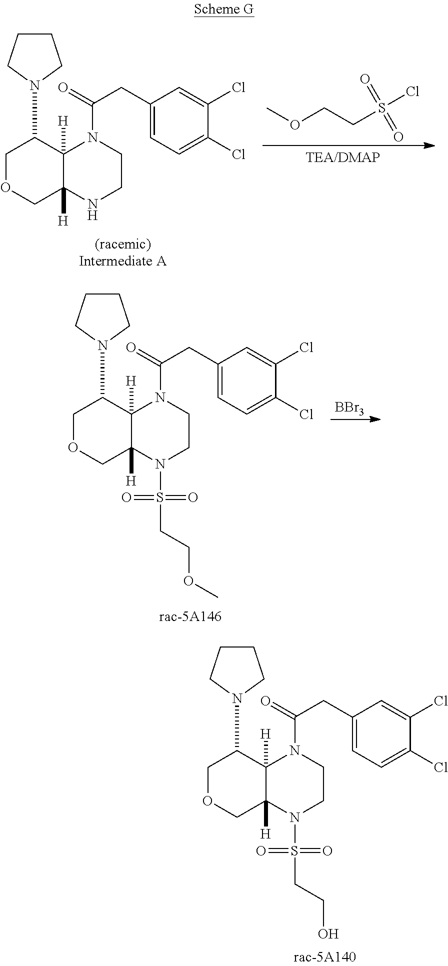

### Step 1

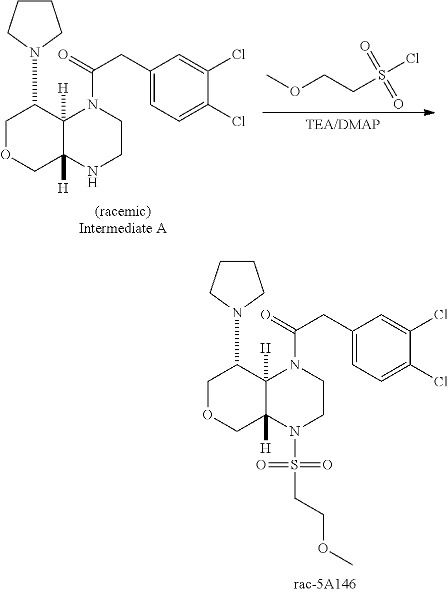

To a solution of 2-methoxyethanesulfonyl chloride (159 mg, 1.00 mmol, 2 eq) in DCM (10 mL) was added 2-(3,4-dichlorophenyl)-1-(8-pyrrolidin-1-yl-2,3,4,4a,5,7,8,8a-octahydropyrano[3,4-b]pyrazin-1-yl)ethanone (200 mg, 502 μmol, 1 eq), TEA (254 mg, 2.51 mmol, 349 μL, 5 eq), and DMAP (6.1 mg, 50 μmol, 0.1 eq). The mixture was stirred for 12 hours at 20° C. The reaction mixture was quenched by addition H₂O (50 mL). The mixture was extracted with DCM (80 mL×2). The combined organic layers were dried over Na₂SO₄, filtered, and concentrated. The residue was purified by preparative-HPLC (acetonitrile/water) to furnish 2-(3,4-dichlorophenyl)-1-[4-(2-methoxyethylsulfonyl)-8-pyrrolidin-1-yl-3,4a,5,7,8,8a-hexahydro-2H-pyrano[3,4-b]pyrazin-1-yl]ethanone (rac-5A146). LCMS m/z: 520.0 [MH⁺]. 1H NMR (400 MHz, methanol-d4) δ 7.42-7.48 (m, 2H), 7.21 (d, J=8.19, 1.59 Hz, 1H), 4.00-4.13 (m, 3H), 3.7-3.94 (m, 4H), 3.44-3.76 (m, 8H), 3.34-3.42 (m, 4H), 3.25-3.30 (m, 1H), 2.62-2.77 (m, 4H), 1.62-1.74 (m, 4H).

### Step 2

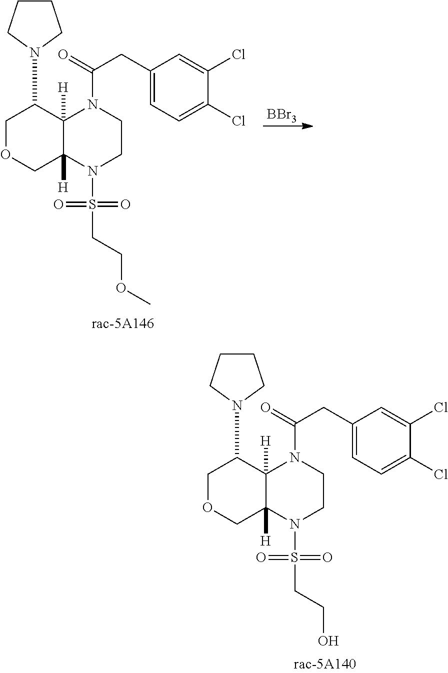

To the solution of 2-(3,4-dichlorophenyl)-1-[4-(2-methoxyethylsulfonyl)-8-pyrrolidin-1-y1-3,4a,5,7,8,8a-hexahydro-2H-pyrano[3,4-b]pyrazin-1-yl]ethanone (100 mg, 134 μmol, 1 eq) in DCM (5 mL) was added dropwise BBr₃ (202 mg, 807 μmol, 78 μL, 6 eq) at −64° C. over 15 minutes. The mixture was stirred at 20° C. for 0.5 hr. The mixture was quenched with MeOH (5 ml) and concentrated under reduced pressure. The residue was purified by preparative-HPLC (acetonitrile/water) to furnish 2-(3,4-dichlorophenyl)-1-[4-(2-hydroxyethylsulfonyl)-8-pyrrolidin-1-yl-3,4a,5,7,8,8a-hexahydro-2H-pyrano[3,4-b]pyrazin-1-yl]ethanone (rac-5A140). LCMS m/z: 506.2 [MH+]. 1H NMR (400 MHz, methanol-d4) δ 7.49-7.42 (m, 2H), 7.24-7.18 (m, 1H), 4.14-4.02 (m, 2H), 3.93-3.72 (m, 7H), 3.71-3.50 (m, 4H), 3.46-3.33 (m, 3H), 3.23 (t, J=6.13 Hz, 2H), 2.79-2.66 (m, 3H), 1.74-1.64 (m, 4H).

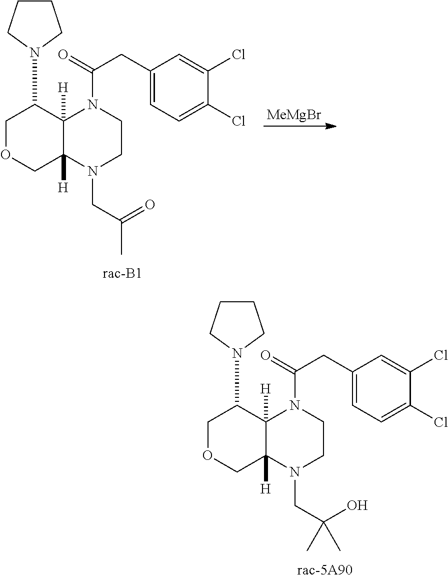 [SMILES: "MeMgBr" ; CC(=O)CN1CCN(C(=O)Cc2ccc(Cl)c(Cl)c2)[C@H]2[C@H]1COC[C@@H]2N1CCCC1 ; CC(C)(O)CN1CCN(C(=O)Cc2ccc(Cl)c(Cl)c2)[C@H]2[C@H]1COC[C@@H]2N1CCCC1]

Methylmagnesium bromide (3 M, 3.96 mL, 20 eq) was added to a solution of 1-[1-[2-(3,4-dichlorophenyl)acetyl]-8-pyrrolidin-1-yl-3,4a,5,7,8,8a-hexahydro-2H-pyrano[3,4-b]pyrazin-4-yl]propan-2-one (270 mg, 594 μmol, 1 eq) in THF (3 mL) dropwise at −40° C. under N₂. The mixture was stirred at −20-−40° C. for 3 hours. The mixture was warmed to 0° C., and stirred at that temperature for 0.5 hr under N₂. The reaction mixture was quenched by addition H₂O (50 mL) at 0° C. The mixture was extracted with ethyl acetate (50 mL×3). The combined organic layers were dried over Na₂SO₄, filtered, and concentrated. The residue was purified by preparative-HPLC (column: Luna C18 100 mm×30 mm×5μ; mobile phase: [water(0.04% HCl)-ACN]; B%: 5%-35%, 12 minute gradient) to furnish 2-(3,4-dichlorophenyl)-1-[4-(2-hydroxy-2-methyl-propyl)-8-pyrrolidin-1-yl-3,4a,5,7,8,8a-hexahydro-2H-pyrano[3,4-b]pyrazin-1-yl]ethanone (rac-5A90). LCMS m/z: 470.2 [MH+]. 1H NMR (400 MHz, methanol-d4) δ 7.59 (s, 1H), 7.50 (d, J=8.19 Hz, 1H), 7.32 (d, J=7.95 Hz, 1H), 4.19-4.69 (m, 5H), 3.87-4.17 (m, 5H), 3.56-3.84 (m, 6H), 3.34-3.40 (m, 1H), 3.18-3.30 (m, 2H), 1.95-2.14 (m, 4H), 1.34 (s, 6H).

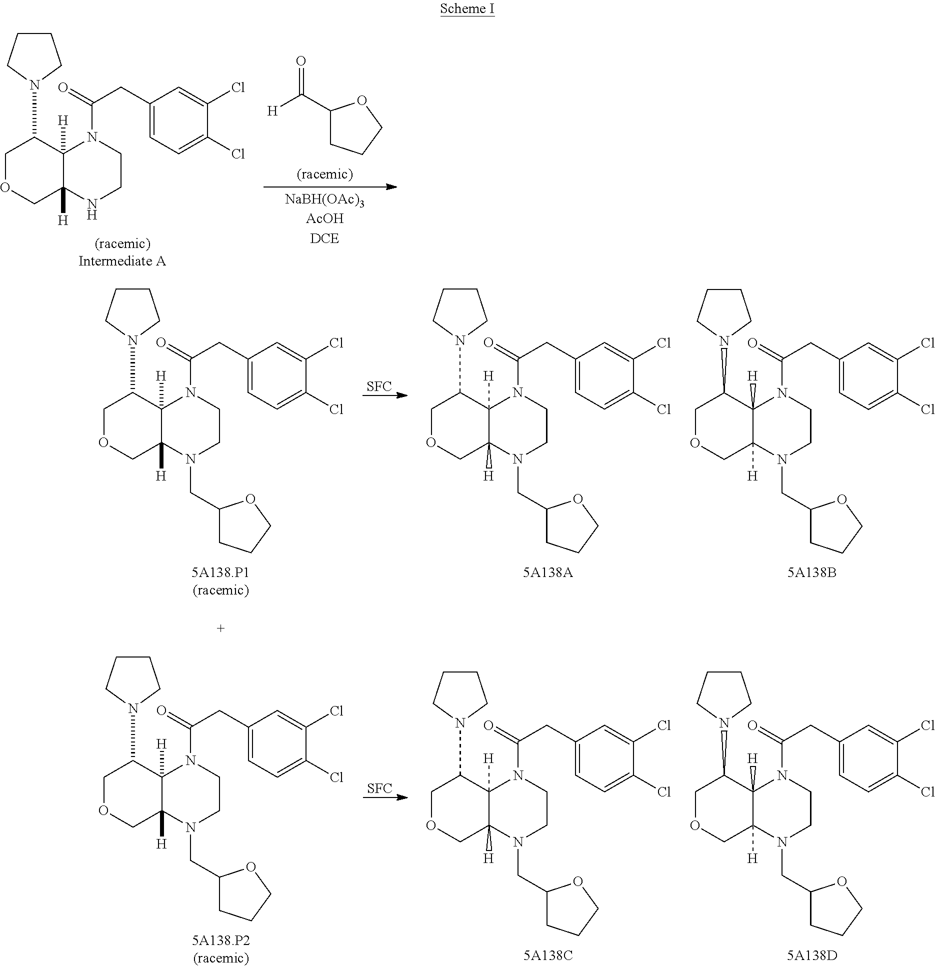

### Step 1

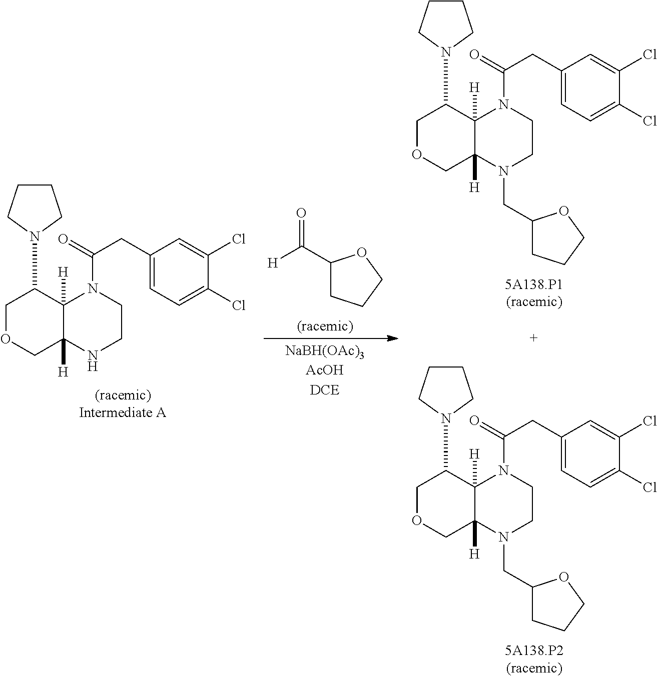

To a solution of 2-(3,4-dichlorophenyl)-1-(8-pyrrolidin-1-yl-2,3,4,4a,5,7,8,8a-octahydropyrano[3,4-b]pyrazin-1-yl)ethanone (700 mg, 1.76 mmol, 1 eq) in DCE (10 mL) was added 1,3-dioxolane-4-carbaldehyde (1.08 g, 5.27 mmol, 3 eq) and HOAc (317 mg, 5.27 mmol, 302 μL, 3 eq). After stirring the mixture at 20° C. for 0.5 hr, NaBH(OAc)₃ (1.12 g, 5.27 mmol, 3 eq) was added. The mixture was stirred at 20° C. for 11.5 hours. The reaction mixture was quenched with H₂O (30 mL) and stirred for 10 min. The mixture was extracted with DCM (50 mL×3). The combined organic layers were washed with brine (50 mL), dried with anhydrous Na₂SO₄, and filtered. The filtrate was concentrated. The residue was purified by preparative-HPLC (column: Phenomenex Luna C18 200 mm×40 mm×10μm; mobile phase: [water(0.05% HCl)-ACN]; B%: 10%-30%, 10 min) to furnish 5A138.P1 (first eluting) and 5A138.P2 (second eluting). 5A138.P1: 1H NMR (400 MHz, methanol-d4) δ 7.58 (d, J=1.83 Hz, 1H), 7.49 (d, J=8.31 Hz, 1H), 7.32 (d, J=8.25, 1.90 Hz, 1H), 4.26-4.54 (m, 5H), 3.89-4.06 (m, 5H), 3.53-3.87 (m, 8H), 3.33-3.43 (m, 2H), 3.16-3.29 (m, 2H), 1.89-2.21 (m, 7H), 1.54-1.68 (m, 1H). 5A138.P2: 1H NMR (400 MHz, methanol-d4) δ 7.58 (d, J=1.34 Hz, 1H), 7.49 (d, J=8.19 Hz, 1H), 7.32 (d, J=8.19, 1.47 Hz, 1H), 4.52 (d, J=5.99 Hz, 1H), 4.23-4.44 (m, 4H), 3.80-4.05 (m, 8H), 3.61-3.79 (m, 4H), 3.33-3.53 (m, 3H), 3.20 (d, J=13.69, 9.90 Hz, 1H), 1.81-2.23 (m, 8H), 1.52-1.66 (m, 1H).

### Step 2

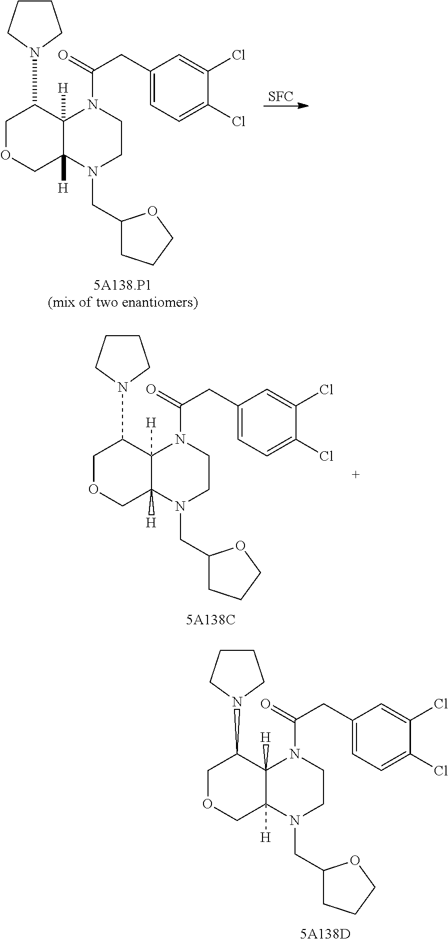

5A138.P1 was separated by SFC (column: DAICEL CHIRALCEL OJ) (250 mm×30 mm×10 μm); mobile phase: [0.1% NH₃H₂O MeOH]; B%: 25%-25%, 6 min) to give 5A138C (Peak 2: retention time—1.44 minutes) and 5A138D ((Peak 1: retention time—1.15 minutes). LCMS m/z: 482.2 [MH⁺].

### Step 3

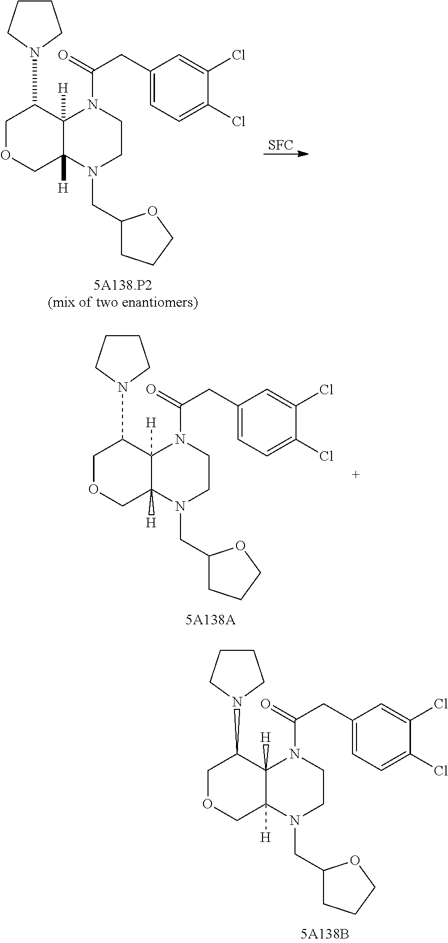

5A138.P2 was separated by SFC (column: DAICEL CHIRALCEL OJ (250 mm×30 mm×10 μm); mobile phase: [0.1% NH₃H₂O MeOH]; B%: 25%-25%, 6 min) to give 5A138A (Peak 2: retention time—1.48 minutes) and 5A138B ((Peak 1: retention time—1.12 minutes). LCMS m/z: 482.2 [MH⁺].

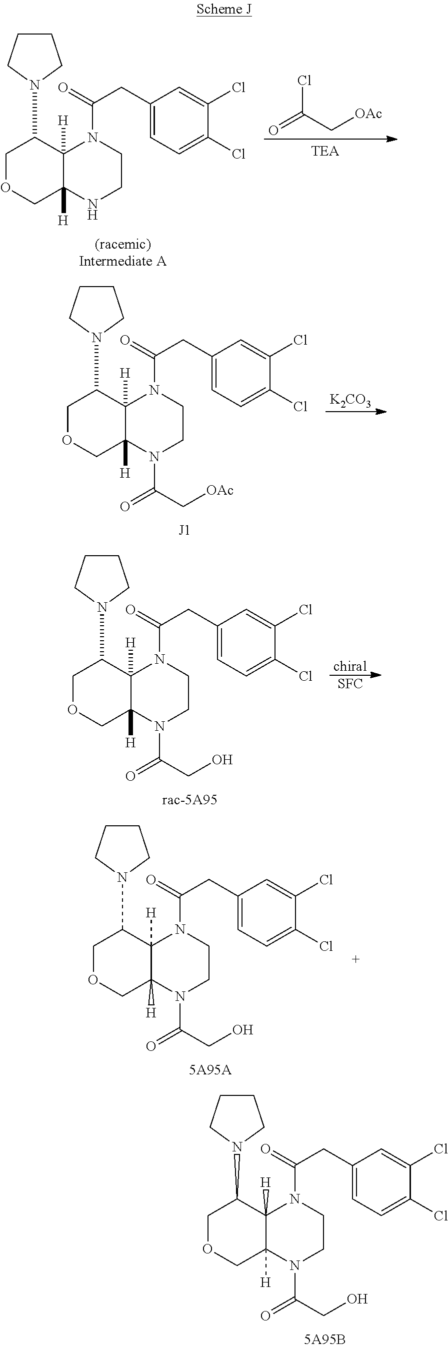

### Step 1

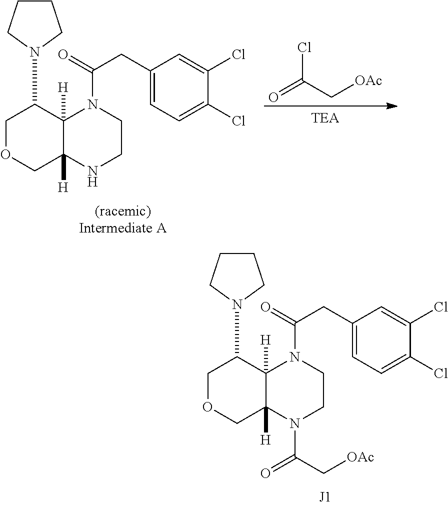

To a solution of 2-(3,4-dichlorophenyl)-1-(8-pyrrolidin-1-yl-2,3,4,4a,5,7,8,8a-octahydropyrano[3,4-b]pyrazin-1-yl)ethanone (250 mg, 628 μmol, 1 eq) in DCM (15 mL) was added Et₃N (318 mg, 3.14 mmol, 437 μL, 5 eq) and (2-chloro-2-oxoethyl)acetate (257 mg, 1.88 mmol, 202 μL, 3 eq) under N₂. The mixture was stirred at 20° C. for 3 hr. The reaction mixture was quenched with H₂O (20 mL). The resulting mixture was extracted with DCM (20 mL×2). The combined organic layers were dried over Na₂SO₄, filtered, and concentrated. The crude residue was used in the next step without further purification.

### Step 2

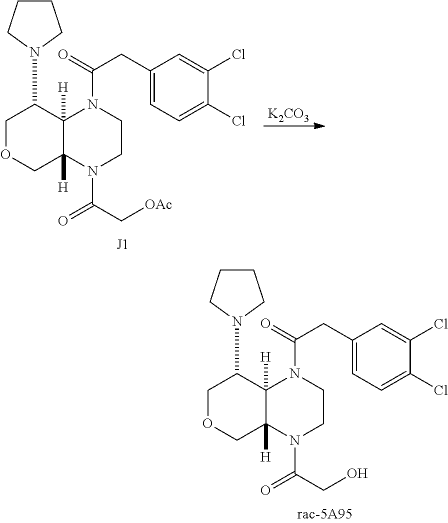

To a solution of [2-[1-[2-(3,4-dichlorophenyl)acetyl]-8-pyrrolidin-1-yl-3,4a,5,7,8,8a-hexahydro-2H-pyrano[3,4-b]pyrazin-4-yl]-2-oxo-ethyl] acetate (320 mg, 642 μmol, 1 eq) in MeOH (10 mL) was added K₂CO₃ (444 mg, 3.21 mmol, 5 eq). The mixture was stirred at 30° C. for 1 hr. The reaction mixture was concentrated under reduced pressure. The residue was triturated with H₂O (50 mL) and filtered. The solid residue was purified by preparative-HPLC (column: Kromasil 150 mm×25 mm×10 μm; mobile phase: [water(0.04% NH₃H₂O+10 mM NH₄HCO₃)-ACN]; B%: 20%-40%, 10 min) to furnish rac-5A95. 1H NMR (400 MHz, methanol-d4) δ 7.48 (s, 1H), 7.48 (d, J=8.19 Hz, 1H), 7.44 (d, J=1.83 Hz, 1H), 7.18 (d, J=8.19, 1.96 Hz, 1H), 4.62 (s, 1H), 3.99-4.16 (m, 5H), 3.59-3.84 (m, 4H), 3.25-3.43 (m, 3H), 2.60-2.82 (m, 1H), 2.60-2.82 (m, 3H), 1.71 (s, 4H).

### Step 3

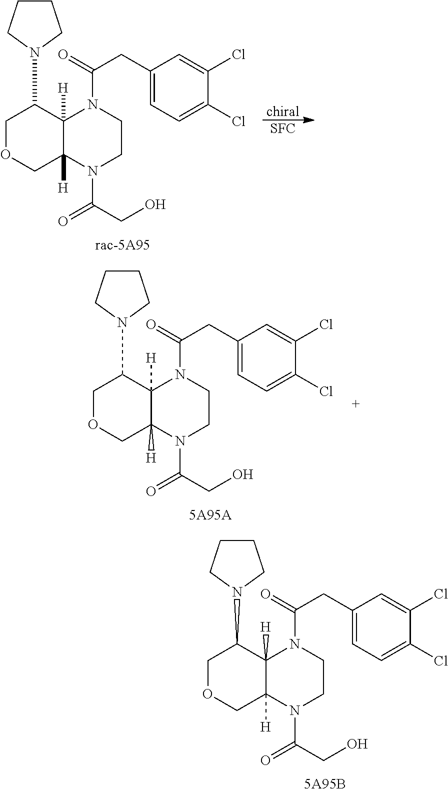

rac-5A95 2-(3,4-dichlorophenyl)-1-[4-(2-hydroxyacetyl)-8-pyrrolidin-1-yl-3,4a,5,7,8,8a-hexahydro-2H-pyrano[3,4-b]pyrazin-1-yl]ethanone was separated by chiral SFC (column: DAICEL CHIRALPAK AD (250 mm×30 mm×10 μm); mobile phase: [0.1% NH₃H₂O MeOH]; B%: 45%-45%, 10 min) to give: 5A95A (Peak 1: retention time—2.72 minutes) and 5A95B (Peak 2: retention time—3.66 minutes). LCMS m/z: 456.2 [MH⁺].

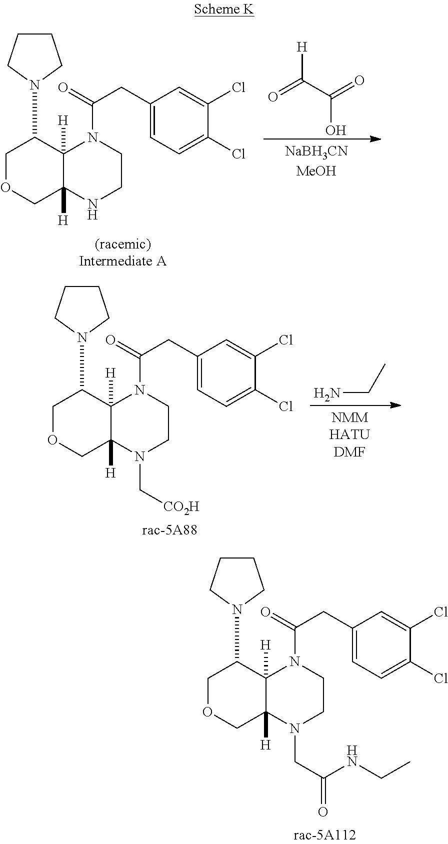

### Step 1

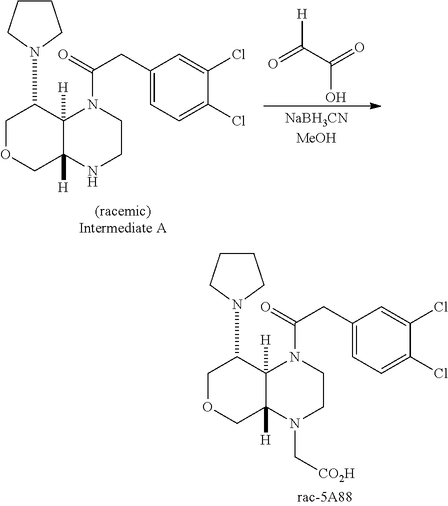

Intermediate A (100 mg, 251 □mol, 1 eq) and 2-oxoacetic acid (37 mg, 502 □mol, 28 μL, 2 eq) were dissolved in MeOH (20 mL). Sodium cyanoborohydride (32 mg, 502 μmol, 2 eq) was added. The resulting mixture was stirred at 25° C. under N₂ for 12 hrs. The reaction mixture was concentrated under reduced pressure to remove solvent. The residue was purified by preparative-TLC (SiO₂, DCM: MeOH=10:1) to furnish rac-5A88. LCMS m/z: 456.1 [MH⁺]. 1H NMR (400 MHz, methanol-d4) δ=7.50-7.45 (m, 2H), 7.23 (dd, J=2.0, 8.3 Hz, 1H), 4.74 (dt, J=4.3, 10.6 Hz, 1H), 4.29 (dd, J=4.3, 11.2 Hz, 1H), 4.18 (dd, J=4.7, 10.7 Hz, 1H), 4.06 (td, J=3.5, 14.6 Hz, 1H), 3.91-3.79 (m, 2H), 3.65-3.44 (m, 6H), 3.40-3.34 (m, 3H), 3.27-3.16 (m, 1H), 2.89 (td, J=3.5, 11.5 Hz, 1H), 2.81-2.72 (m, 1H), 2.12-1.92 (m, 4H).

### Step 2

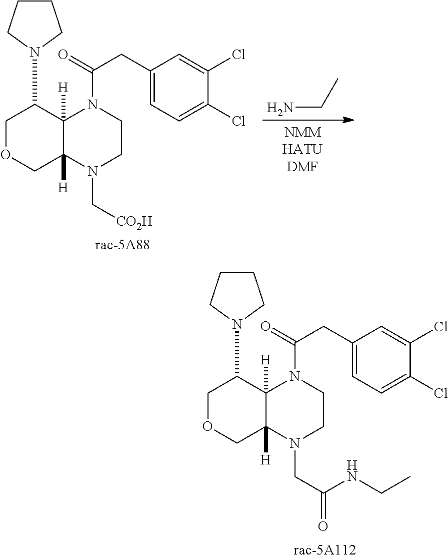

Rac-5A88 (10 mg, 22 μmol, 1 eq), HATU (17 mg, 44 μmol, 2 eq) and NMM (11 mg, 110 μmol, 12 μL, 5 eq) were dissolved in DMF (2 mL). The resulting mixture was degassed and purged with N₂ (3 cycles). The resulting mixture was stirred at 25° C. for 0.3 hr under a N₂ atmosphere. Ethyl amine (10 mg, 22 μmol, 1 eq) was added, and the resulting mixture was stirred at 40° C. for 11.7 hours under a N₂ atmosphere. The reaction mixture was concentrated under reduced pressure to remove solvent. The residue was diluted with EtOAc (5 mL) and H₂O (3 mL). The mixture was extracted with EtOAc (5 mL×3). The combined organic layers were washed with brine (5 mL×3), dried over Na₂SO₄, and filtered. The filtrate was concentrated under reduced pressure. The residue was purified by preparative-HPLC (TFA condition: column: Luna C₁₈ 100 mm×30 mm×5μ; mobile phase: [water (0.1% TFA)-ACN]; B%: 10%-35%, 4 min gradient) to furnish rac-5A112. LCMS m/z: 483.2 [MH⁺]. 1H NMR (400 MHz, methanol-d4) δ=7.52-7.47 (m, 2H), 7.25 (dd, J=2.1, 8.3 Hz, 1H), 4.78 (dt, J=4.2, 10.8 Hz, 1H), 4.31 (dd, J=4.3, 11.1 Hz, 1H), 4.17 (dd, J=5.1, 11.2 Hz, 1H), 4.10-4.04 (m, 1H), 3.86 (d, J=7.9 Hz, 2H), 3.71-3.59 (m, 2H), 3.56-3.46 (m, 3H), 3.29-3.25 (m, 2H), 3.25-3.20 (m, 3H), 3.01-2.83 (m, 3H), 2.55 (dt, J=3.3, 11.1 Hz, 1H), 2.11-1.96 (m, 4H), 1.13 (t, J=7.3 Hz, 3H).

The following racemic analogs were resolved into the corresponding antipodes using the conditions outlined in Table C.

TABLE C

| | | Retention Time | |
|---|---|---|---|
| Racemic Analog | SFC conditions | Isomer A | Isomer B |
| 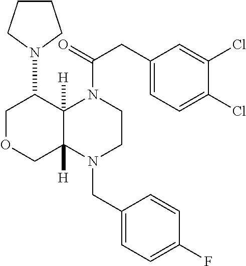 [SMILES: O=C(Cc1ccc(Cl)c(Cl)c1)N1CCN(Cc2ccc(F)cc2)[C@@H]2COC[C@H](N3CCCC3)[C@H]21] | column: DAICEL CHIRALPAK IC (250 mm × 30 mm, 10 μm); mobile phase: [0.1% NH₃H₂O IPA]; B %: 0-20%, 15 min | 5A136A 2.2 min | 5A136B 2.45 min |
| rac-5A136 | | | |
| 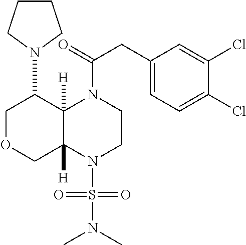 [SMILES: CN(C)S(=O)(=O)N1CCN(C(=O)Cc2ccc(Cl)c(Cl)c2)[C@H]2[C@H]1COC[C@@H]2N1CCCC1] | column: DAICEL CHIRALPAK AD-H (250 mm × 30 mm, 5 μm); mobile phase: [0.1% NH₃H₂O IPA]; B %: 50%-50%, 12 min | 5A56A 1.89 min | 5A56B 2.40 min |
| rac-5A56 | | | |
| 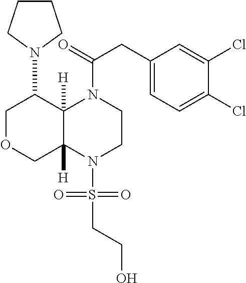 [SMILES: O=C(Cc1ccc(Cl)c(Cl)c1)N1CCN(S(=O)(=O)CCO)[C@@H]2COC[C@H](N3CCCC3)[C@H]21] | column: DAICEL CHIRALPAK AD (250 mm × 50 mm, 10 μm); mobile phase: [0.1% NH₃H₂O IPA]; B %: 42%-42%, 10 min | 5A140A 1.85 min | 5A140B 2.09 min |
| rac-5A140 | | | |
| 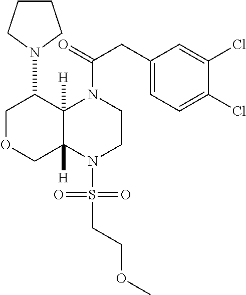 [SMILES: COCCS(=O)(=O)N1CCN(C(=O)Cc2ccc(Cl)c(Cl)c2)[C@H]2[C@H]1COC[C@@H]2N1CCCC1] | column: DAICEL CHIRALPAK AD-H (250 mm × 30 mm, 5 μm); mobile phase: [0.1% NH₃H₂O MeOH]; B %: 50%-50%, 12 min | 5A146A 0.92 min | 5A146B 2.24 min |
| rac-5A146 | | | |
| 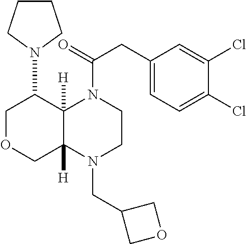 [SMILES: O=C(Cc1ccc(Cl)c(Cl)c1)N1CCN(CC2COC2)[C@@H]2COC[C@H](N3CCCC3)[C@H]21] | column: DAICEL CHIRALPAK AY (250 mm × 30 mm, 20 μm); mobile phase: [0.1% NH₃H₂O EtOH]; B %: 45%-45%, 15 min | 5A83A 1.95 min | 5A83B 2.37 min |
| rac-5A83 | | | |
| 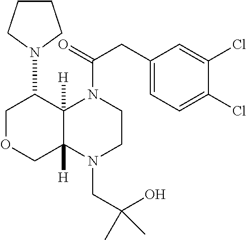 [SMILES: CC(C)(O)CN1CCN(C(=O)Cc2ccc(Cl)c(Cl)c2)[C@H]2[C@H]1COC[C@@H]2N1CCCC1] | column: DAICEL CHIRALPAK IC (250 mm × 30 mm, 5 μm); mobile phase: [0.1% NH₃H₂O MeOH]; B %: 42%-42%, 8 min | 5A90A 1.56 min | 5A90B 2.02 min |
| rac-5A90 | | | |
| 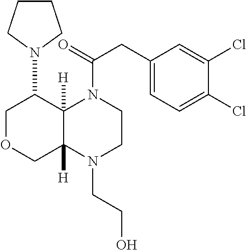 [SMILES: O=C(Cc1ccc(Cl)c(Cl)c1)N1CCN(CCO)[C@@H]2COC[C@H](N3CCCC3)[C@H]21] | column: DAICEL CHIRALPAK AD (250 mm × 50 mm, 10 μm); mobile phase: [0.1% NH₃H₂O IPA]; B %: 41%-41%, 5.6 min) | 5A74A 2.04 min | 5A74B 2.79 min |
| rac-5A74 | | | |
| 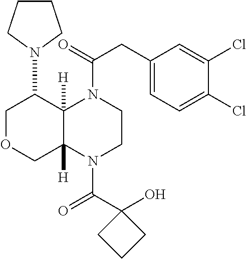 [SMILES: O=C(Cc1ccc(Cl)c(Cl)c1)N1CCN(C(=O)C2(O)CCC2)[C@@H]2COC[C@H](N3CCCC3)[C@H]21] | column: DAICEL CHIRALPAK AD (250 mm × 30 mm, 10 μm); mobile phase: [0.1% NH₃H₂O EtOH]; B %: 44%-44%, 10 min | 5A128A 1.99 min | 5A128B 2.28 min |
| rac-5A128 | | | |
| 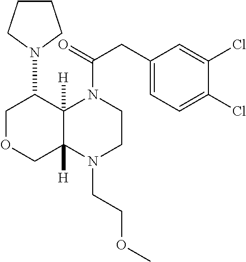 [SMILES: COCCN1CCN(C(=O)Cc2ccc(Cl)c(Cl)c2)[C@H]2[C@H]1COC[C@@H]2N1CCCC1] | column: DAICEL CHIRALCEL OJ (250 mm × 30 mm, 10 μm); mobile phase: [0.1% NH₃H₂O MeOH]; B %: 45%-45%, 6 min) | 5A141A 1.12 min | 5A141B 1.48 min |
| rac-5A141 | | | |
| 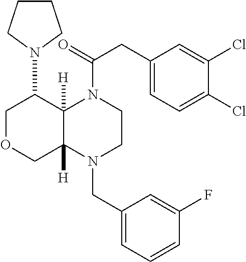 [SMILES: O=C(Cc1ccc(Cl)c(Cl)c1)N1CCN(Cc2cccc(F)c2)[C@@H]2COC[C@H](N3CCCC3)[C@H]21] | column: DAICEL CHIRALCEL OJ (250 mm × 30 mm, 10 μm); mobile phase: [0.1% NH₃H₂O IPA]; B %: 45%-45%, 6 min) | 5A145A 1.98 min | 5A145B 2.36 min |
| rac-5A145 | | | |
| 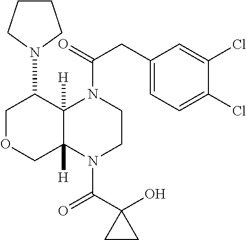 [SMILES: O=C(Cc1ccc(Cl)c(Cl)c1)N1CCN(C(=O)C2(O)CC2)[C@@H]2COC[C@H](N3CCCC3)[C@H]21] | column: DAICEL CHIRALPAK ADH (250 mm × 30 mm, 5 μm); mobile phase: [0.1% NH₃H₂O EtOH]; B %: 40%-40%, 8.5 min | 5A126A 1.87 min | 5A126B 2.13 min |
| rac-5A126 | | | |
| 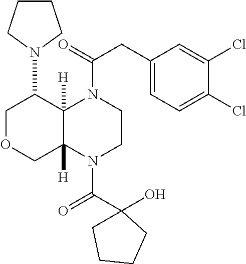 [SMILES: O=C(Cc1ccc(Cl)c(Cl)c1)N1CCN(C(=O)C2(O)CCCC2)[C@@H]2COC[C@H](N3CCCC3)[C@H]21] | column: DAICEL CHIRALPAK AS (250 mm × 50 mm, 10 μm); mobile phase: [0.1% NH₃H₂O MeOH]; B %: 42%-42%, 3 min | 5A125A 1.48 min | 5A125B 1.25 min |
| rac-5A125 | | | |
| 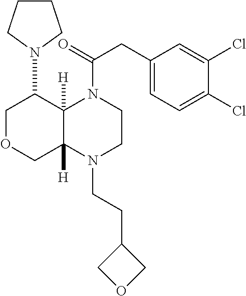 [SMILES: O=C(Cc1ccc(Cl)c(Cl)c1)N1CCN(CCC2COC2)[C@@H]2COC[C@H](N3CCCC3)[C@H]21] | column: DAICEL CHIRALPAK IC (250 mm × 50 mm, 10 μm); mobile phase: [0.1% NH₃H₂O MeOH]; B %: 40%-40%, 6 min | 5A142A 2.41 min | 5A142B 2.91 min |
| rac-5A142 | | | |
| 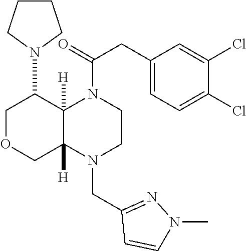 [SMILES: Cn1ccc(CN2CCN(C(=O)Cc3ccc(Cl)c(Cl)c3)[C@H]3[C@H]2COC[C@@H]3N2CCCC2)n1] | column: DAICEL CHIRALPAK AD-H (250 mm × 30 mm, 5 μm); mobile phase: [0.1% NH₃H₂O IPA]; B %: 50%-50%, 12 min | 5A135A 1.85 min | 5A135B 2.35 min |
| rac-5A135 | | | |
| 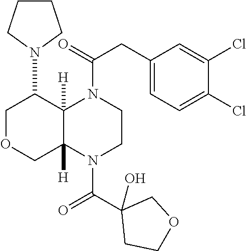 [SMILES: O=C(Cc1ccc(Cl)c(Cl)c1)N1CCN(C(=O)C2(O)CCOC2)[C@@H]2COC[C@H](N3CCCC3)[C@H]21] | column: DAICEL CHIRALPAK AD (250 mm × 30 mm, 10 μm); mobile phase: [0.1% NH₃H₂O IPA]; B %: 45%-45%, 11 min | 5A139A 1.99 min | 5A139B 2.18 min |
| rac-5A139 | | | |
| 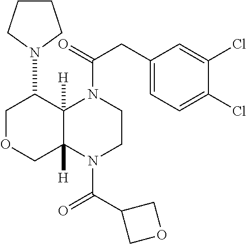 [SMILES: O=C(Cc1ccc(Cl)c(Cl)c1)N1CCN(C(=O)C2COC2)[C@@H]2COC[C@H](N3CCCC3)[C@H]21] | column: DAICEL CHIRALPAK IC (250 mm × 30 mm, 5 μm); mobile phase: [Neutral-MeOH]; B %: 40%-40%, 11 min | 5A133A 6.40 min | 5A133B 8.35 min |
| rac-5A133 | | | |

The compounds shown in Table 1 were made as shown above and tested in the following screens.

Tritiated U69,593 binding for KOP-R membranes; tritiated DAMGO binding for MOP-R membranes; and tritiated DPDPE binding for DOP-R membranes:

Membranes from cells stably expressing kappa, mu or delta opioid receptor constructs (PathHunter U2OS hOPRK1, CHO-K1 rOPRM1 and CHO-K1 OPRD1 β-arrestin cell line, DiscoverX, Fremont, Calif., USA) were used. Cells were scraped from tissue culture plates, homogenized with a tissue tearor homogenizer in membrane buffer (10 mM Tris, 100 mM NaCl, and 1 mM EDTA; pH 7.4), and centrifuged at 20,000 g for 30 minutes at 4° C. and frozen at −80° C. until use. Prior to use, the pellets were resuspended in binding buffer (50 mm Tris, 100 mm NaCl, pH 7.4), homogenized with a dounce homogenizer and 50 μg incubated with 1.0 nM of the appropriate tritiated ligand ([³H]U69,593, [³H]DAMGO or [³H]DPDPE for kappa, mu or delta binding, respectively) and the appropriate concentration of compound for 60 minutes at 30° C. Membranes with bound tritiated ligand were collected on Whatman GF/B filter paper (Brandel, Gaithersburg, Md., USA) utilizing a Brandel harvester. Bound tritiated ligand was quantified using a TriCarb-2900TR scintillation counter (Packard, Downers Grove, Ill., USA) following addition of 4 ml ReadySafe scintillation fluid (Beckman Coulter, Indianapolis, Ind., USA).

### GTPgammaS

Membranes from U2OS cells stably expressing human kappa opioid receptors were used. Cells were scraped from tissue culture plates, homogenized with a tissue tearor homogenizer in membrane buffer (10 mM Tris, 100 mM NaCl, and 1 mM EDTA; pH 7.4), and centrifuged at 20,000 g for 30 minutes at 4° C. and frozen at −80° C. until use. Prior to use, the pellets were resuspended in assay buffer (50 mm Tris, 100 mm NaCl, 5 mM MgCl₂, and 1 mM EDTA; pH 7.4) and homogenized with a dounce homogenizer and 50 μg incubated with 0.1 nM [³⁵ S]GTPγS, 10 nM GDP, and the appropriate concentration of agonist for 20 minutes at 30° C. To test inhibition, all samples were incubated with 100 nM U69,593 as well as the appropriate concentration of compound. Membranes with bound [³⁵ S]GTPγS were collected on Whatman GF/B filter paper (Brandel, Gaithersburg, Md., USA) utilizing a Brandel harvester. Bound [³⁵ S]GTPγS was quantified using a TriCarb-2900TR scintillation counter (Packard, Downers Grove, Ill., USA) following addition of 4 mL ReadySafe scintillation fluid (Beckman Coulter, Indianapolis, Ind., USA). “No Stim” indicates that there was no stimulation in this assay.

### β₂-Arrestin

Experiments can be conducted using the PathHunter Detection Kit obtained from DiscoverX. Cells stably expressing kappa, mu or delta opioid receptor constructs (PathHunter U2OS hOPRK1, CHO-K1 rOPRM1 and CHO-K1 OPRD1 β-arrestin cell line, DiscoverX, Fremont, Calif., USA) are plated in 96- or 384-well plates. Cells are stimulated with the compounds for 90 minutes at 37° C. To test inhibition, all samples were incubated with 100 nM U69,593 as well as the appropriate concentration of compound. Cells are then incubated for 60 minutes in the presence of galoctosidase substrate, yielding chemiluminescent product. Chemiluminescence is measured using a Synergy Neo microplate reader (BioTek, Winooski, Vt., USA). Antagonism assays are done in the same manner, in the presence of 300 nM U69,593, 1 μM DAMGO or 1 μM DPDPE for KOP-R, MOP-R or DOP-R assays, respectively.

Representative compounds of the invention were tested in the foregoing screens with the following results shown in Table D.

TABLE D

| | | GTPγS EC₅₀ | |
|---|---|---|---|
| | Binding IC₅₀ | (nM) | |
| Example | (nM) | (% efficacy) | |
| 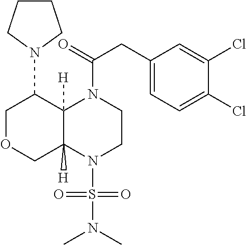 [SMILES: CN(C)S(=O)(=O)N1CCN(C(=O)Cc2ccc(Cl)c(Cl)c2)[C@H]2[C@H]1COC[C@@H]2N1CCCC1] | 7.2 | 11 | (77%) |
| 5A56A | | | |
| 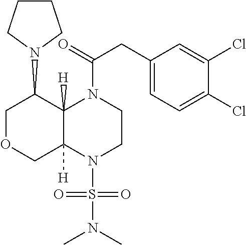 [SMILES: CN(C)S(=O)(=O)N1CCN(C(=O)Cc2ccc(Cl)c(Cl)c2)[C@H]2[C@H](N3CCCC3)COC[C@@H]21] | 113 | 123 | (122%) |
| 5A56B | | | |
| 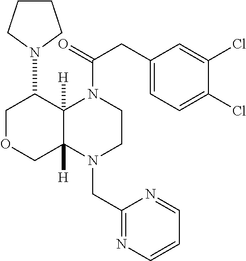 [SMILES: O=C(Cc1ccc(Cl)c(Cl)c1)N1CCN(Cc2ncccn2)[C@@H]2COC[C@H](N3CCCC3)[C@H]21] | 191 | 94 | (114%) |
| rac-5A69 | | | |
| 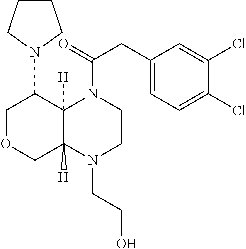 [SMILES: O=C(Cc1ccc(Cl)c(Cl)c1)N1CCN(CCO)[C@@H]2COC[C@H](N3CCCC3)[C@H]21] | 15 | 21 | (95%) |
| 5A74A | | | |
| 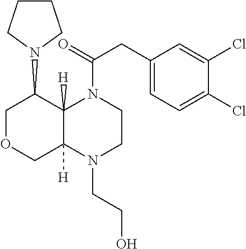 [SMILES: O=C(Cc1ccc(Cl)c(Cl)c1)N1CCN(CCO)[C@H]2COC[C@@H](N3CCCC3)[C@@H]21] | 13 | 12 | (95%) |
| 5A74B | | | |
| 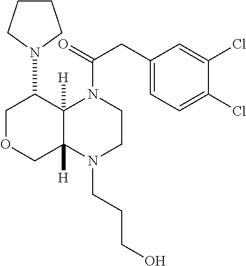 [SMILES: O=C(Cc1ccc(Cl)c(Cl)c1)N1CCN(CCCO)[C@@H]2COC[C@H](N3CCCC3)[C@H]21] | 60 | 39 | (89%) |
| rac-5A75 | | | |
| 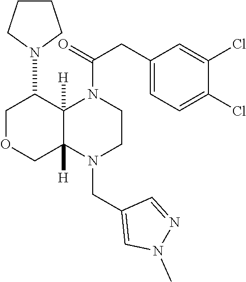 [SMILES: Cn1cc(CN2CCN(C(=O)Cc3ccc(Cl)c(Cl)c3)[C@H]3[C@H]2COC[C@@H]3N2CCCC2)cn1] | 177 | 107 | (82%) |
| rac-5A79 | | | |
| 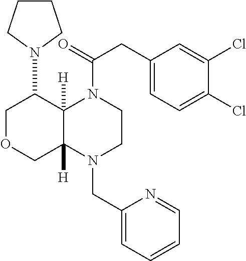 [SMILES: O=C(Cc1ccc(Cl)c(Cl)c1)N1CCN(Cc2ccccn2)[C@@H]2COC[C@H](N3CCCC3)[C@H]21] | 67 | 64 | (94%) |
| rac-5A80 | | | |
| 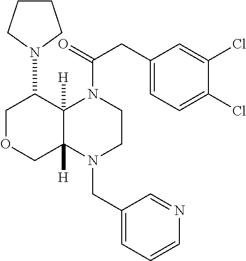 [SMILES: O=C(Cc1ccc(Cl)c(Cl)c1)N1CCN(Cc2cccnc2)[C@@H]2COC[C@H](N3CCCC3)[C@H]21] | 18.7 | 7.4 | (95%) |
| rac-5A81 | | | |
| 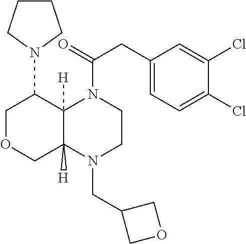 [SMILES: O=C(Cc1ccc(Cl)c(Cl)c1)N1CCN(CC2COC2)[C@@H]2COC[C@H](N3CCCC3)[C@H]21] | 117 | 199 | (141%) |
| 5A83A | | | |
| 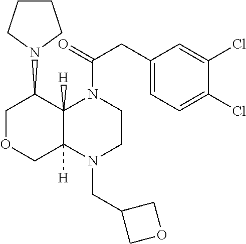 [SMILES: O=C(Cc1ccc(Cl)c(Cl)c1)N1CCN(CC2COC2)[C@H]2COC[C@@H](N3CCCC3)[C@@H]21] | 10 | 24 | (113%) |
| 5A83B | | | |
| 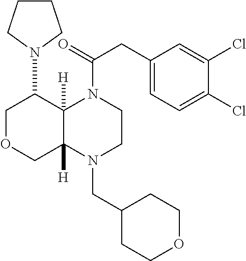 [SMILES: O=C(Cc1ccc(Cl)c(Cl)c1)N1CCN(CC2CCOCC2)[C@@H]2COC[C@H](N3CCCC3)[C@H]21] | 423 | 136 | (84%) |
| rac-5A84 | | | |
| 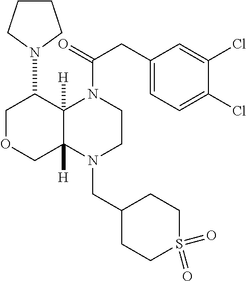 [SMILES: O=C(Cc1ccc(Cl)c(Cl)c1)N1CCN(CC2CCS(=O)(=O)CC2)[C@@H]2COC[C@H](N3CCCC3)[C@H]21] | 1072 | 320 | (100%) |
| rac-5A85 | | | |
| 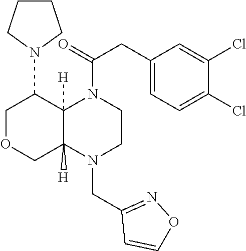 [SMILES: O=C(Cc1ccc(Cl)c(Cl)c1)N1CCN(Cc2ccon2)[C@@H]2COC[C@H](N3CCCC3)[C@H]21] | 1.7 | 1 | (99%) |
| 5A86A | | | |
| 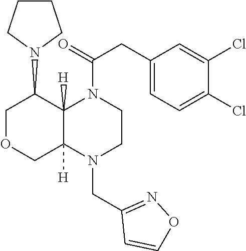 [SMILES: O=C(Cc1ccc(Cl)c(Cl)c1)N1CCN(Cc2ccon2)[C@H]2COC[C@@H](N3CCCC3)[C@@H]21] | 23 | 23 | (79%) |
| 5A86B | | | |
| 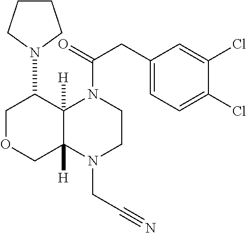 [SMILES: N#CCN1CCN(C(=O)Cc2ccc(Cl)c(Cl)c2)[C@H]2[C@H]1COC[C@@H]2N1CCCC1] | 16 | 20 | (115%) |
| rac-5A87 | | | |
| 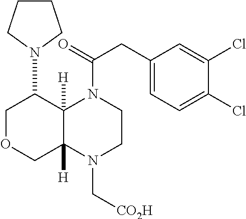 [SMILES: O=C(O)CN1CCN(C(=O)Cc2ccc(Cl)c(Cl)c2)[C@H]2[C@H]1COC[C@@H]2N1CCCC1] | 1995 | 2486 | (83%) |
| rac-5A88 | | | |
| 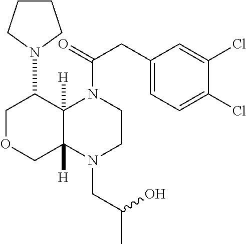 [SMILES: CC(O)CN1CCN(C(=O)Cc2ccc(Cl)c(Cl)c2)[C@H]2[C@H]1COC[C@@H]2N1CCCC1] | 14 | 34 | (93%) |
| 5A89 | | | |
| (mix of 4 isomers) | | | |
| 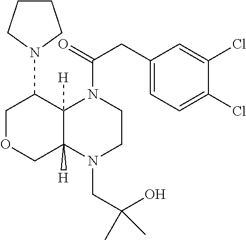 [SMILES: CC(C)(O)CN1CCN(C(=O)Cc2ccc(Cl)c(Cl)c2)[C@H]2[C@H]1COC[C@@H]2N1CCCC1] | 28 | 22 | (101%) |
| 5A90A | | | |
| 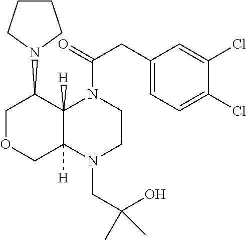 [SMILES: CC(C)(O)CN1CCN(C(=O)Cc2ccc(Cl)c(Cl)c2)[C@H]2[C@H](N3CCCC3)COC[C@@H]21] | 11 | 5.4 | (68%) |
| 5A90B | | | |
| 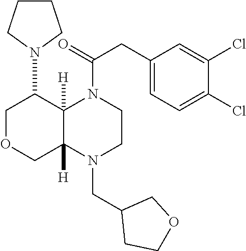 [SMILES: O=C(Cc1ccc(Cl)c(Cl)c1)N1CCN(CC2CCOC2)[C@@H]2COC[C@H](N3CCCC3)[C@H]21] | 110 | 46 | (80%) |
| 5A93 | | | |
| (mix of 4 isomers) | | | |
| 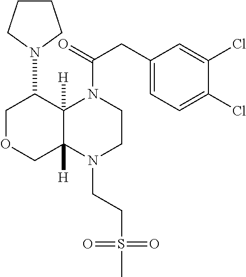 [SMILES: CS(=O)(=O)CCN1CCN(C(=O)Cc2ccc(Cl)c(Cl)c2)[C@H]2[C@H]1COC[C@@H]2N1CCCC1] | 177 | 27 | (109%) |
| rac-5A94 | | | |
| 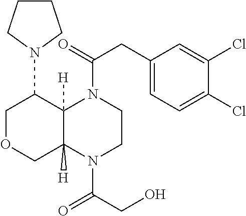 [SMILES: O=C(Cc1ccc(Cl)c(Cl)c1)N1CCN(C(=O)CO)[C@@H]2COC[C@H](N3CCCC3)[C@H]21] | 46 | 21 | (87%) |
| 5A95A | | | |
| 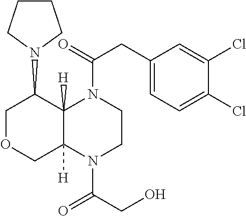 [SMILES: O=C(Cc1ccc(Cl)c(Cl)c1)N1CCN(C(=O)CO)[C@H]2COC[C@@H](N3CCCC3)[C@@H]21] | 308 | 202 | (78%) |
| 5A95B | | | |
| 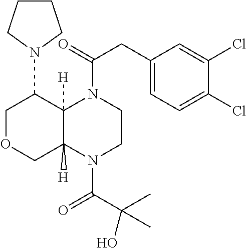 [SMILES: CC(C)(O)C(=O)N1CCN(C(=O)Cc2ccc(Cl)c(Cl)c2)[C@H]2[C@H]1COC[C@@H]2N1CCCC1] | 31 | 17 | (64%) |
| 5A96A | | | |
| 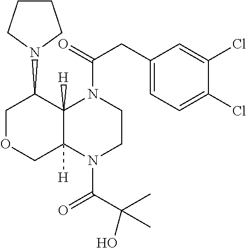 [SMILES: CC(C)(O)C(=O)N1CCN(C(=O)Cc2ccc(Cl)c(Cl)c2)[C@H]2[C@H](N3CCCC3)COC[C@@H]21] | 63 | 64 | (113%) |
| 5A96B | | | |
| 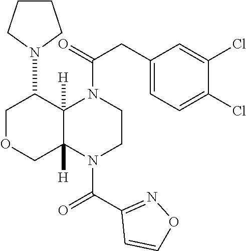 [SMILES: O=C(Cc1ccc(Cl)c(Cl)c1)N1CCN(C(=O)c2ccon2)[C@@H]2COC[C@H](N3CCCC3)[C@H]21] | 12 | 0.8 | (88%) |
| rac-5A97 | | | |
| 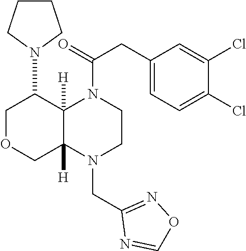 [SMILES: O=C(Cc1ccc(Cl)c(Cl)c1)N1CCN(Cc2ncon2)[C@@H]2COC[C@H](N3CCCC3)[C@H]21] | 22 | 1.9 | (86%) |
| rac-5A100 | | | |
| 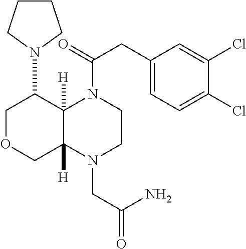 [SMILES: NC(=O)CN1CCN(C(=O)Cc2ccc(Cl)c(Cl)c2)[C@H]2[C@H]1COC[C@@H]2N1CCCC1] | 90 | 37 | (96%) |
| rac-5A102 | | | |
| 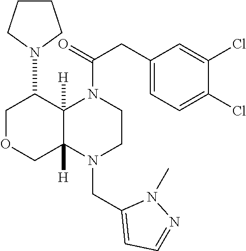 [SMILES: Cn1nccc1CN1CCN(C(=O)Cc2ccc(Cl)c(Cl)c2)[C@H]2[C@H]1COC[C@@H]2N1CCCC1] | 78 | 9 | (74%) |
| rac-5A104 | | | |
| 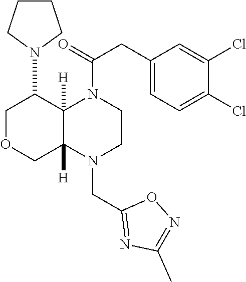 [SMILES: Cc1noc(CN2CCN(C(=O)Cc3ccc(Cl)c(Cl)c3)[C@H]3[C@H]2COC[C@@H]3N2CCCC2)n1] | 37 | 19 | (82%) |
| rac-5A105 | | | |
| 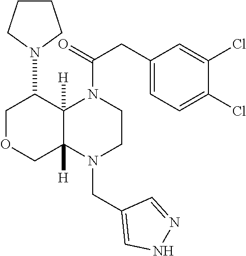 [SMILES: O=C(Cc1ccc(Cl)c(Cl)c1)N1CCN(Cc2cn[nH]c2)[C@@H]2COC[C@H](N3CCCC3)[C@H]21] | 11 | 3.9 | (112%) |
| rac-5A107 | | | |
| 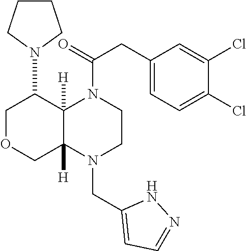 [SMILES: O=C(Cc1ccc(Cl)c(Cl)c1)N1CCN(Cc2ccn[nH]2)[C@@H]2COC[C@H](N3CCCC3)[C@H]21] | 16 | 26 | (98%) |
| rac-5A108 | | | |
| 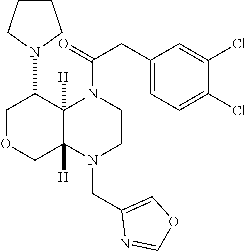 [SMILES: O=C(Cc1ccc(Cl)c(Cl)c1)N1CCN(Cc2cocn2)[C@@H]2COC[C@H](N3CCCC3)[C@H]21] | 61 | ND | |
| rac-5A109 | | | |
| 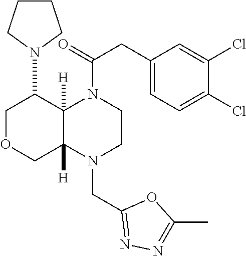 [SMILES: Cc1nnc(CN2CCN(C(=O)Cc3ccc(Cl)c(Cl)c3)[C@H]3[C@H]2COC[C@@H]3N2CCCC2)o1] | 76 | 40 | (91%) |
| rac-5A118 | | | |
| 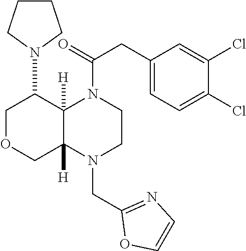 [SMILES: O=C(Cc1ccc(Cl)c(Cl)c1)N1CCN(Cc2ncco2)[C@@H]2COC[C@H](N3CCCC3)[C@H]21] | 6.5 | 9.8 | (146%) |
| rac-5A111 | | | |
| 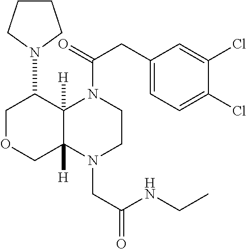 [SMILES: CCNC(=O)CN1CCN(C(=O)Cc2ccc(Cl)c(Cl)c2)[C@H]2[C@H]1COC[C@@H]2N1CCCC1] | 254 | 85 | (75%) |
| rac-5A112 | | | |
| 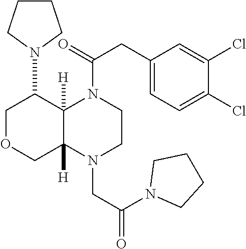 [SMILES: O=C(CN1CCN(C(=O)Cc2ccc(Cl)c(Cl)c2)[C@H]2[C@H]1COC[C@@H]2N1CCCC1)N1CCCC1] | 198 | 70 | (92%) |
| rac-5A113 | | | |
| 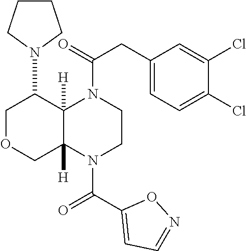 [SMILES: O=C(Cc1ccc(Cl)c(Cl)c1)N1CCN(C(=O)c2ccno2)[C@@H]2COC[C@H](N3CCCC3)[C@H]21] | 34 | 17 | (86%) |
| rac-5A121 | | | |
| 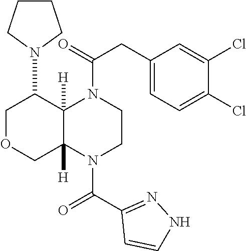 [SMILES: O=C(Cc1ccc(Cl)c(Cl)c1)N1CCN(C(=O)c2cc[nH]n2)[C@@H]2COC[C@H](N3CCCC3)[C@H]21] | 78 | 31 | (79%) |
| rac-5A122 | | | |
| 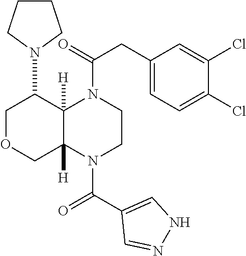 [SMILES: O=C(Cc1ccc(Cl)c(Cl)c1)N1CCN(C(=O)c2cn[nH]c2)[C@@H]2COC[C@H](N3CCCC3)[C@H]21] | 159 | 39 | (62%) |
| rac-5A123 | | | |
| 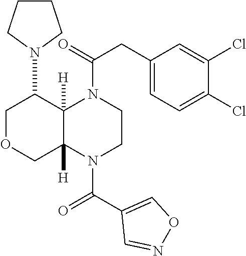 [SMILES: O=C(Cc1ccc(Cl)c(Cl)c1)N1CCN(C(=O)c2cnoc2)[C@@H]2COC[C@H](N3CCCC3)[C@H]21] | 433 | ND | |
| rac-5A124 | | | |
| 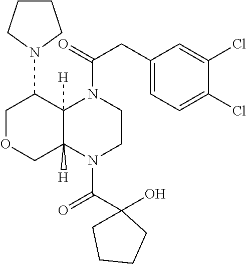 [SMILES: O=C(Cc1ccc(Cl)c(Cl)c1)N1CCN(C(=O)C2(O)CCCC2)[C@@H]2COC[C@H](N3CCCC3)[C@H]21] | 258 | 58 | (95%) |
| 5A125A | | | |
| 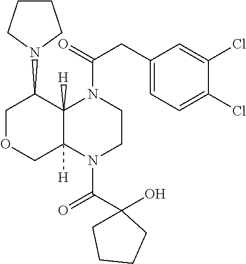 [SMILES: O=C(Cc1ccc(Cl)c(Cl)c1)N1CCN(C(=O)C2(O)CCCC2)[C@H]2COC[C@@H](N3CCCC3)[C@@H]21] | 53 | 35 | (81%) |
| 5A125B | | | |
| 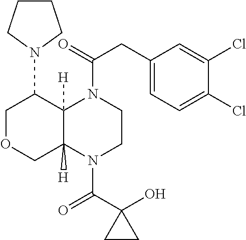 [SMILES: O=C(Cc1ccc(Cl)c(Cl)c1)N1CCN(C(=O)C2(O)CC2)[C@@H]2COC[C@H](N3CCCC3)[C@H]21] | 27 | 16 | (98%) |
| 5A126A | | | |
| 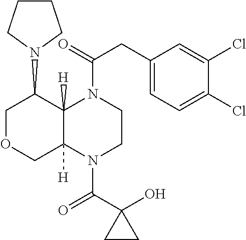 [SMILES: O=C(Cc1ccc(Cl)c(Cl)c1)N1CCN(C(=O)C2(O)CC2)[C@H]2COC[C@@H](N3CCCC3)[C@@H]21] | 28 | 17 | (107%) |
| 5A126B | | | |
| 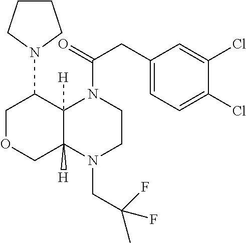 [SMILES: CC(F)(F)CN1CCN(C(=O)Cc2ccc(Cl)c(Cl)c2)[C@H]2[C@H]1COC[C@@H]2N1CCCC1] | 6.2 | 5.3 | (110%) |
| 5A127A | | | |
| 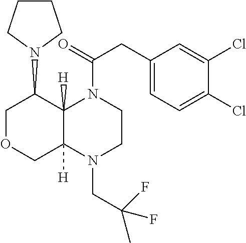 [SMILES: CC(F)(F)CN1CCN(C(=O)Cc2ccc(Cl)c(Cl)c2)[C@H]2[C@H](N3CCCC3)COC[C@@H]21] | 20 | 24 | (88%) |
| 5A127B | | | |
| 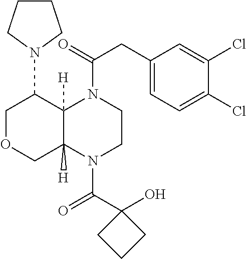 [SMILES: O=C(Cc1ccc(Cl)c(Cl)c1)N1CCN(C(=O)C2(O)CCC2)[C@@H]2COC[C@H](N3CCCC3)[C@H]21] | 103 | ND | |
| 5A128A | | | |
| 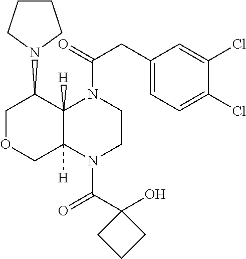 [SMILES: O=C(Cc1ccc(Cl)c(Cl)c1)N1CCN(C(=O)C2(O)CCC2)[C@H]2COC[C@@H](N3CCCC3)[C@@H]21] | 19 | ND | |
| 5A128B | | | |
| 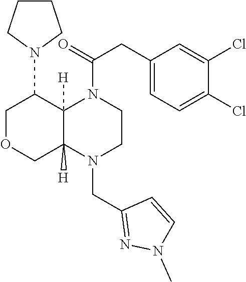 [SMILES: Cn1ccc(CN2CCN(C(=O)Cc3ccc(Cl)c(Cl)c3)[C@H]3[C@H]2COC[C@@H]3N2CCCC2)n1] | 123 | ND | |
| 5A135A | | | |
| 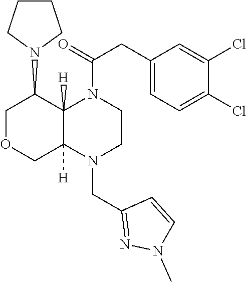 [SMILES: Cn1ccc(CN2CCN(C(=O)Cc3ccc(Cl)c(Cl)c3)[C@H]3[C@H](N4CCCC4)COC[C@@H]32)n1] | 123 | ND | |
| 5A135B | | | |
| 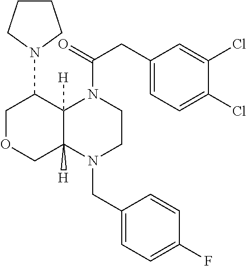 [SMILES: O=C(Cc1ccc(Cl)c(Cl)c1)N1CCN(Cc2ccc(F)cc2)[C@@H]2COC[C@H](N3CCCC3)[C@H]21] | 24 | 7 | (105%) |
| 5A136A | | | |
| 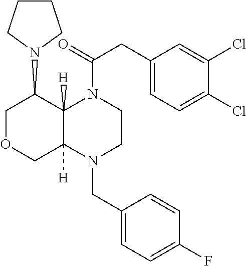 [SMILES: O=C(Cc1ccc(Cl)c(Cl)c1)N1CCN(Cc2ccc(F)cc2)[C@H]2COC[C@@H](N3CCCC3)[C@@H]21] | 6.9 | 74 | (129%) |
| 5A136B | | | |
| 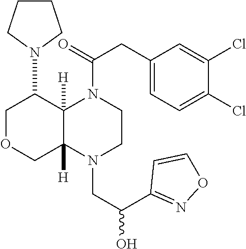 [SMILES: O=C(Cc1ccc(Cl)c(Cl)c1)N1CCN(CC(O)c2ccon2)[C@@H]2COC[C@H](N3CCCC3)[C@H]21] | 9.4 | ND | |
| 5A137 | | | |
| (mix of 4 isomers) | | | |
| 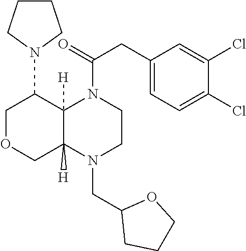 [SMILES: O=C(Cc1ccc(Cl)c(Cl)c1)N1CCN(CC2CCCO2)[C@@H]2COC[C@H](N3CCCC3)[C@H]21] | 101 | ND | |
| 5A138A | | | |
| 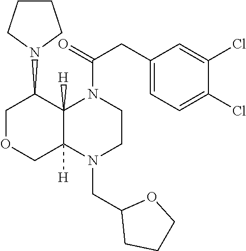 [SMILES: O=C(Cc1ccc(Cl)c(Cl)c1)N1CCN(CC2CCCO2)[C@H]2COC[C@@H](N3CCCC3)[C@@H]21] | 73 | ND | |
| 5A138B | | | |
| 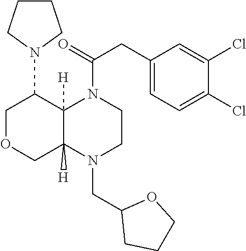 [SMILES: O=C(Cc1ccc(Cl)c(Cl)c1)N1CCN(CC2CCCO2)[C@@H]2COC[C@H](N3CCCC3)[C@H]21] | 31 | ND | |
| 5A138C | | | |
| 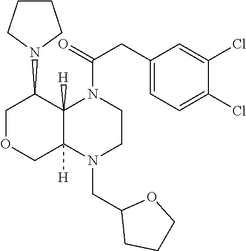 [SMILES: O=C(Cc1ccc(Cl)c(Cl)c1)N1CCN(CC2CCCO2)[C@H]2COC[C@@H](N3CCCC3)[C@@H]21] | 87 | 100 | (102%) |
| 5A138D | | | |
| 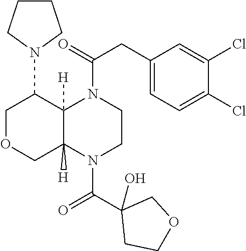 [SMILES: O=C(Cc1ccc(Cl)c(Cl)c1)N1CCN(C(=O)C2(O)CCOC2)[C@@H]2COC[C@H](N3CCCC3)[C@H]21] | 310 | ND | |
| 5A139A | | | |
| 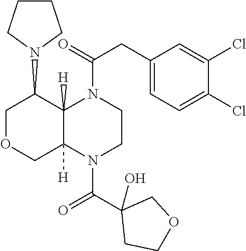 [SMILES: O=C(Cc1ccc(Cl)c(Cl)c1)N1CCN(C(=O)C2(O)CCOC2)[C@H]2COC[C@@H](N3CCCC3)[C@@H]21] | 1705 | ND | |
| 5A139B | | | |
| 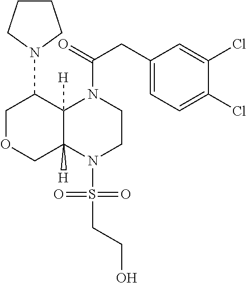 [SMILES: O=C(Cc1ccc(Cl)c(Cl)c1)N1CCN(S(=O)(=O)CCO)[C@@H]2COC[C@H](N3CCCC3)[C@H]21] | 9.5 | 20 | (112%) |
| 5A140A | | | |
| 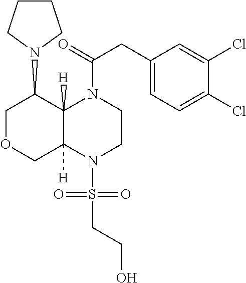 [SMILES: O=C(Cc1ccc(Cl)c(Cl)c1)N1CCN(S(=O)(=O)CCO)[C@H]2COC[C@@H](N3CCCC3)[C@@H]21] | 17 | 22 | (107%) |
| 5A140B | | | |
| 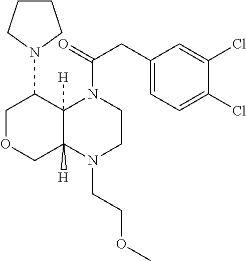 [SMILES: COCCN1CCN(C(=O)Cc2ccc(Cl)c(Cl)c2)[C@H]2[C@H]1COC[C@@H]2N1CCCC1] | 90 | 37 | (96%) |
| 5A141A | | | |
| 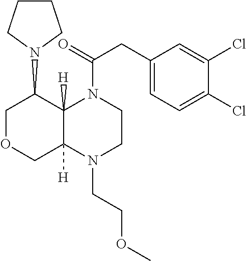 [SMILES: COCCN1CCN(C(=O)Cc2ccc(Cl)c(Cl)c2)[C@H]2[C@H](N3CCCC3)COC[C@@H]21] | 21 | 26 | (103%) |
| 5A141B | | | |
| 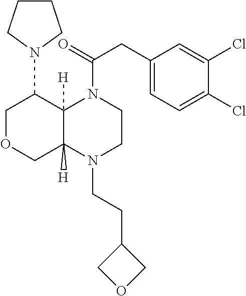 [SMILES: O=C(Cc1ccc(Cl)c(Cl)c1)N1CCN(CCC2COC2)[C@@H]2COC[C@H](N3CCCC3)[C@H]21] | 88 | 70 | (77%) |
| 5A142A | | | |
| 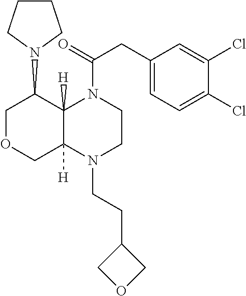 [SMILES: O=C(Cc1ccc(Cl)c(Cl)c1)N1CCN(CCC2COC2)[C@H]2COC[C@@H](N3CCCC3)[C@@H]21] | 1480 | ND | |
| 5A142B | | | |
| 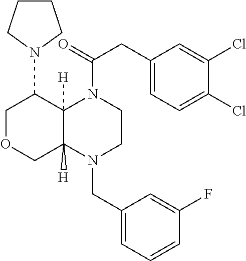 [SMILES: O=C(Cc1ccc(Cl)c(Cl)c1)N1CCN(Cc2cccc(F)c2)[C@@H]2COC[C@H](N3CCCC3)[C@H]21] | 23 | 5 | (95%) |
| 5A145A | | | |
| 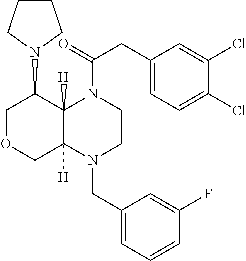 [SMILES: O=C(Cc1ccc(Cl)c(Cl)c1)N1CCN(Cc2cccc(F)c2)[C@H]2COC[C@@H](N3CCCC3)[C@@H]21] | 59 | 9.7 | (91%) |
| 5A145B | | | |
| 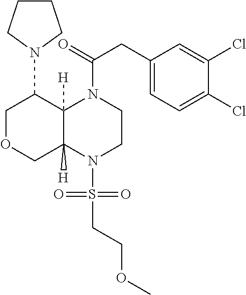 [SMILES: COCCS(=O)(=O)N1CCN(C(=O)Cc2ccc(Cl)c(Cl)c2)[C@H]2[C@H]1COC[C@@H]2N1CCCC1] | 42 | 9.2 | (91%) |
| 5A146A | | | |
| 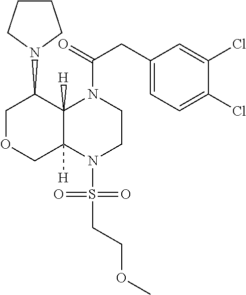 [SMILES: COCCS(=O)(=O)N1CCN(C(=O)Cc2ccc(Cl)c(Cl)c2)[C@H]2[C@H](N3CCCC3)COC[C@@H]21] | 17 | 115 | (127%) |
| 5A146B | | | |

ND = not determined

It has been demonstrated that prolactin release from the pituitary is a reliable biomarker of KOP-r agonism across species. Thus, demonstration of the release of prolactin by a compound which is predicted from in vitro GTPgammaS assays in cell lines expressing KOP-r, in a manner blocked by a selective kappa antagonist, indicates an in vivo KOP-r agonistic effect. The demonstration of differential maximal efficacy in prolactin release compared to the full unbiased agonist U50488-induced release, coupled with submaximal kappa opioid receptor mediated GTPgammaS, indicates that the compound has in vivo partial agonist KOP-r activity.

In the case of rotarod incoordination, kappa agonist effects in this assay reflect kappa-opioid receptor arrestin mediated signaling. This assay is thought to be a sensitive measure of the sedative properties of kappa opioid receptor agonists. Generally, a compound which has reduced efficacy in the coupling of arrestin with the kappa opioid receptor is thought to have a lowered potential for the sedative side effects of kappa opioid receptor ligands Rotarod assays in vivo are employed to confirm this possibility.

### Prolactin

Mice are injected intraperitoneally with the compound to be tested 30 minutes prior to sampling. Trunk blood is collected by rapid decapitation, followed within 2 hours by preparation of serum. Serum prolactin levels are determined using a commercially available enzyme-linked immunoassay (AbCam, Cambridge, UK) following dilution of serum 5-fold in assay buffer.

### Rotarod

Rotarod experiments are conducted with mice using a dedicated rodent rotarod apparatus, with up to five animals tested concurrently (IITC Life Science, Woodland Hills, Calif, USA). Rotarod rotation rate begins at 3 rotations per minute, and ramps to 30 rotations per minute over the course of 300 s, at which time the assay is terminated and animals removed to their home cage. Animals are acclimated to the rotarod on at least two occasions prior to the day of the test. On the day of the test, baseline times for each animal to fall off the rotarod are recorded. Mice are then injected intraperitoneally with vehicle or compound, and rotarod measurements conducted, beginning 0-2 minutes after injection, and then subsequently at select time points thereafter. Animals which fail to remain on the rotarod for at least 150 seconds during baseline testing are removed from the analysis.

The resolved diastereomer pairs 5A90A/5A90B and 5A96A/5A96B were examined in vivo in dose-response prolactin and rotarod assays in male mice to demonstrate central kappa opioid receptor activity. (The closure of laboratories during the covid-19 pandemic delayed the carrying out of conditioned place aversion assays and modulation of cocaine self-administration, so that results were not available by the filing date of this application.)

TABLE E

| | Binding | GTPγS | GTPγS | Arrestin | Arrestin |
|---|---|---|---|---|---|
| | potency | potency | efficacy, | Potency | efficacy, |
| Example | (nM) | (nM) | % | (nM) | % |
| 5A90A | 46.3 ± | 34.7 ± | 91.0 ± | 43187± | 78.1 ± |
| | 14.2 | 15.0 | 15.1 | 17085 | 12.5 |
| 5A90B | 19.3 ± | 8.9 ± | 69.3 ± | 5253 ± | 46.5 ± |
| | 4.9 | 4.0 | 3.7 | 2035 | 1.3 |
| 5A96A | 50.5 ± | 32.8 ± | 77.0 ± | 3760 ± | 53.0 ± |
| | 28.7 | 5.5 | 11.4 | 1958 | 16.1 |
| 5A96B | 104.9 ± | 71.4 ± | 123.5 ± | 24788 ± | 60.7 ± |
| | 44.2 | 7.6 | 10.8 | 10072 | 8.5 |

In an initial experiment, examples 5A90B and 5A96A were examined in the prolactin assay described above at 30 mg/kg and compared to control and 10 mg/kg of the selective kappa-opioid receptor agonist U50,488H as a positive control. U50,488H, 5A90B and 5A96A induced statistically significant serum prolactin levels between 2 and 3.5 ng/mL (p>0.05). In a subsequent dose-response experiment, 5A90B exhibited statistically significant stimulation of prolactin levels at 30 mg/kg (p<0.05) and 90 mg/kg (p<0.005).

In the rotarod protocol described above, 5A96A induced no statistically significant sedative effect up to 90 mg/kg. Example 5A90B exhibited a substantial sedative effect at 90 mg/kg, but not at 30 mg/kg or 10 mg/kg.

1. A compound of Formula I 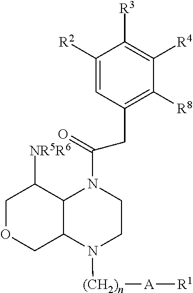
wherein
A is chosen from —(C═O)—, —CH₂—, —CH(OH)—, —(C═O)NH—, —SO₂—, and a direct bond
n is 0, 1, or 2;
R¹ is chosen from cyano, hydroxy(C₁-C₆)hydrocarbyl, (C₁-C₆)oxaalkyl, fluoro(C₁-C₆)alkyl, cyano, —COOH, —SO₂NH(C₁-C₆)hydrocarbyl, —SO₂N[(C₁-C₆)hydrocarbyl]₂, and optionally-substituted heterocyclyl, wherein substituents on said heterocycle are chosen from (C₁-C₇)hydrocarbyl, (C₁-C₃)alkoxy, fluoro(C₁-C₃)alkyl, hydroxy, and oxo;
or, when A is —(C═O)NH—, R¹ may additionally be hydrogen or (C₁-C₆)alkyl;
or, when n is other than zero, R¹ may additionally be —SO₂(C₁-C₆)hydrocarbyl
R², R³, R⁴, and R⁸ are chosen independently from hydrogen, halogen, (C₁-C₄)alkyl, fluoro(C₁-C₄)alkyl, cyano, nitro, —SO₃H and —N⁺HR⁵R⁶; and
R⁵ and R⁶ are chosen from (C₁-C₁₀)hydrocarbyl, optionally substituted with fluoro, or, taken together with the nitrogen to which they are attached, R⁵ and R⁶ form a five-, six- or seven-membered non-aromatic heterocycle, which may be optionally substituted with fluoro or (C₁-C₄)alkyl.

2. A compound according to claim 1 wherein two of R², R³, R⁴, and R⁸ are hydrogen and the remaining two are chosen from hydrogen, halogen, fluoro(C₁-C₄)alkyl, and cyano.

3. A compound according to claim 1 wherein R⁵ and R⁶ form a five-, six- or seven-membered non-aromatic heterocycle, which may be optionally substituted with fluoro or (C₁-C₄)alkyl.

4. A compound according to claim 1 wherein R¹ is optionally-substituted heterocyclyl.

5. A compound according to claim 4 wherein n is zero and A is —CH₂— or —C(═O)—.

6. A compound according to claim 4 wherein n is one and A is —CH₂— or —CH(OH)—.

7. A compound according to claim 4 wherein said optionally-substituted heterocyclyl is chosen from tetrahydrofuran, isoxazole, oxazole, oxetane, pyrazole, pyridine, oxadiazole, pyrimidine, pyrrolidine, tetrahydropyran, and tetrahydrothiopyran 1,1-dioxide.

8. A compound according to claim 7 wherein said heterocycle is unsubstituted or substituted with methyl and/or hydroxy.

9. A compound according to claim 8 wherein said heterocycle is chosen from tetrahydrofuran, oxetane, and tetrahydropyran substituted with hydroxy at the position of attachment of said heterocycle to A.

10. A compound according to claim 8 wherein said heterocycle is chosen from isoxazole, oxazole, pyrazole, pyridine, oxadiazole, pyrimidine, and pyrrolidine unsubstituted or substituted with methyl.

11. A compound according to claim 1 wherein R¹ is fluoro(C₃-C₁₀)hydrocarbyl.

12. A compound according to claim 11 wherein n is one, A is a direct bond and R1 is chosen from mono-, di-, or trifluoro(C₂-C₆)alkyl and fluorophenyl.

13. A compound according to claim 1 wherein R¹ is —SO₂N[(C₁-C₆)alkyl]₂.

14. A compound according to claim 1 wherein R¹ is hydroxy(C₁-C₆)alkyl or hydroxy(C₃-C₆)cycloalkyl

15. A compound according to claim 14 wherein R¹ is chosen from hydroxymethyl, hydroxyethyl, hydroxypropyl, hydroxycyclopropyl, hydroxycyclobutyl, and hydroxycyclopentyl.

16. A compound according to claim 1 wherein R¹ is methoxy(C₁-C₆)alkyl.

17. A compound according to claim 1 wherein R¹ is chosen from cyano, —C(═O)NH₂, and —COOH.

18. A compound according to claim 1 wherein R¹ is chosen from —SO₂CH₃, —SO₂(CH₂)ₘOH, and —SO₂(CH₂)mOCH₃, wherein m is two or three.

19. A compound according to claim 1 wherein n is two.

20. A compound according to claim 1 wherein n is one.

21. A compound according to claim 13 or 18 wherein n is zero.

22. A compound according to claim 1 wherein A is a direct bond.

23. A compound according to claim 1 wherein A is —(C═O)—.

24. A compound according to claim 1 wherein A is —CH₂—.

25. A compound according to claim 1 wherein the ring junction of the octahydro-1H-pyrano[3,4-b]pyrazine is trans and —NR⁵R⁶ is cis to its adjacent hydrogen at the ring junction.

26. A compound according to claim 1 wherein —NR⁵R⁶ is 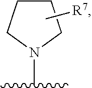
wherein R⁷ is chosen from hydrogen, fluoro and (C₁-C₃)alkyl.

27. A compound according to claim 1, wherein R² and R⁸ are hydrogen, and R³ and R⁴ are halogen or trifluoromethyl.

28. A method for activating a kappa opioid receptor, comprising contacting a kappa opioid receptor with a compound according to claim 1.

29. A method for treating addiction, comprising administering to a patient a compound according to claim 1.

30. A method according to claim 29 wherein said addiction is an addiction to cocaine.

31. A method according to claim 29 wherein said addiction is an addiction to alcohol.

32. A method for treating a mood disorder, comprising administering to a patient a compound according to claim 1.

33. A pharmaceutical composition comprising a pharmaceutically acceptable carrier and a compound according to claim 1.
